(19) United States
(12) Patent Application Publication
(10) Pub. No.: US 2024/0110183 A1
(43) Pub. Date: Apr. 4, 2024

(54) ANTIFIBROTIC AND ANTITUMOR ACTIVITY OF CD63 BLOCKADE

(71) Applicants: Gerlinde WERNIG, Woodside, CA (US); Tristan LERBS, Redwood City, CA (US); Lu CUL, Stanford, CA (US); Qiwen DENG, Stanford, CA (US); Cristabelle DE SOUZA, Stanford, CA (US); The Board of Trustees of the Leland Stanford Junior University, Stanford, CA (US)

(72) Inventors: Gerlinde Wernig, Woodside, CA (US); Tristan Lerbs, Redwood City, CA (US); Lu Cui, Stanford, CA (US); Qiwen Deng, Stanford, CA (US); Cristabelle De Souza, Stanford, CA (US)

(21) Appl. No.: 18/266,790

(22) PCT Filed: Dec. 21, 2021
(86) PCT No.: PCT/US2021/064691

§ 371 Date: Jun. 12, 2023

Related U.S. Application Data
(60) Provisional application No. 63/128,512, filed on Dec. 21, 2020.

(51) Int. Cl. C12N 15/113; C07K 16/28; C12N 9/22

(52) U.S. Cl. CPC C12N 15/113; C07K 16/2896; C12N 9/22; C12N 2310/20

(57) ABSTRACT

Methods are provided for the treatment of cancer and/or fibrosis. It is shown that there is increased CD63 expression both in lung cancer and pulmonary fibrosis, sarcoma, skin fibrosis, liver cancer and liver cirrhosis, NASH and NHFLD, and kidney fibrosis from hypertension and other etiologies. Inhibiting CD63 increases phagocytosis of lung cancer cells and lung fibroblasts; and can eliminates tumor cells and pathogenic fibrosis.

### CROSS REFERENCE TO RELATED APPLICATION

The present application is a 371 and claims the benefit of and priority to PCT/US2021/064691, filed Dec. 21, 2021, which claims priority to U.S. Provisional Patent Application No. 63/128,512, filed Dec. 21, 2020, the entire disclosure of which is hereby.

### STATEMENT OF GOVERNMENT SUPPORT

This invention was made with Government support under contract NIH:HL143143 awarded by the National Institution of Health. The Government has certain rights in the invention.

### INCORPORATION BY REFERENCE OF SEQUENCE LISTING PROVIDED AS A TEXT FILE

A Sequence Listing is provided herewith in a text file, (S20-364_STAN-1793WOSeqListing_ST25), created on Dec. 21, 2021, and having a size of 10000 bytes. The contents of the text file are incorporated herein by reference in its entirety.

### BACKGROUND

Fibrosis, defined by the excessive accumulation of extracellular matrix components (ECM) in and around inflamed or damaged tissue, is associated with several inflammatory conditions. In these situations, normal tissue repair response turns into an irreversible fibrotic response through dysregulation of response to stress or injury. Fibrosis can lead to permanent scarring, organ malfunction and, ultimately, death, as seen in end-stage liver disease, kidney disease, idiopathic pulmonary fibrosis (IPF), retinal fibrosis, and heart failure from cardiac fibrosis. Fibrosis also influences tumor invasion and metastasis, chronic graft rejection and the pathogenesis of many progressive myopathies.

In 2018, more than two million patients were diagnosed with lung cancer worldwide and 1.7 million succumbed to their disease. Risk factors include smoking and increased age, but also underlying lung diseases such as idiopathic pulmonary fibrosis (IPF). Despite recent therapeutic advances, especially on the field of immune-oncology, overall survival remains poor.

Tetraspanins are a group of proteins with four transmembrane domains. Among its members are, among others, CD9, CD63, CD81, and CD151. They participate in protein trafficking and are part of intra- and extracellular vesicles. Extracellular vesicles (EV) can fuse with target cells, thereby releasing their content and supporting tumor progression. In regard of the prognostic relevance of tetraspanins in cancers, results have been inconclusive. Tetraspanins are found on different cells, including immune cells and macrophages.

Fibrosis remains a significant medical challenge. The development of therapies is of interest, and addressed herein.

### SUMMARY

The present invention provides compositions and methods useful for prevention or treatment of fibrosis and/or cancer via inhibition of CD63 pathways. It is shown that there is increased CD63 expression both in lung cancer and pulmonary fibrosis, skin fibrosis, liver cancer and liver cirrhosis, NASH and NHFLD, and kidney fibrosis from hypertension and other etiologies. Inhibiting CD63 increases phagocytosis of CD63 expressing cells, for example lung cancer cells, lung fibroblasts, fibrotic skin cells, liver cancer cells, fibrotic liver cells, etc.; and can eliminates tumor cells and pathogenic fibrosis. In some embodiments, inhibition of CD63 pathways comprises administering to a human subject in need thereof an effective dose of an anti-CD63 antibody, where the dose is effective to prevent or treat fibrosis. In some embodiments, inhibition of CD63 pathways comprises administering to a human subject in need thereof an effective dose of an anti-CD63 antibody, where the dose is effective to prevent or treat cancer.

In some embodiments the fibrosis is a pulmonary fibrosis, such as idiopathic pulmonary fibrosis, and the like. In some embodiments, the fibrosis is selected from skin fibrosis, liver cirrhosis, non-alcoholic steatohepatitis (NASH), non-alcoholic fatty liver disease (NHFLD), and kidney fibrosis. When the fibrosis is kidney fibrosis, the kidney fibrosis may be caused by hypertension, diabetes, obstruction, or infection. In some embodiments the fibrosis is associated with cancer and tumor growth, i.e. tumor related tissue fibrosis, including without limitation lung cancer, liver cancer, skin cancer, sarcoma, osteosarcoma, etc. In other embodiments the fibrosis is associated with chronic inflammation or injury.

An anti-CD63 agent for use in the methods of the invention blocks CD63 signaling. For example, the agent may block interactions of CD63 with its counter-receptors (ligands). In some embodiments an anti-CD63 agent interferes with binding between CD63 present on the fibrotic or cancer cell and co-receptors present on a phagocytic cell, e.g. integrins, etc. Such methods increase phagocytosis of the fibrotic cell. Suitable anti-CD63 agents anti-CD63 antibodies, small molecules, soluble receptors, and the like, where the term antibodies encompasses antibody fragments, immunoglobulin single variable domains (ISVs), and variants thereof, as known in the art. In some embodiments the anti-CD63 agent is an anti-CD63 antibody. In some embodiments, the anti-CD63 antibody is anti-CD63 clone H5C6, produced by Novus.

An anti-CD63 agent for use in the methods of the invention is a therapeutic gene therapy vector. In some embodiments, the therapeutic gene therapy vector is an AAV virus comprising a therapeutic sequence. In some embodiments, the therapeutic vector comprises a CRISPR/Cas9 system and at least one single guide RNA (sgRNA) directed to a CD63 gene for deletion. In some embodiments, the therapeutic vector comprises short hairpin RNA (shRNA) targeting CD63 gene.

In some embodiments, the anti-CD63 agent is administered locally at the site of the cancer or fibrotic tissue. In some embodiments, the anti-CD63 agent is administered systemically.

Compositions and kits for practicing the methods and/or for use with the systems of the disclosure are also provided.

### BRIEF DESCRIPTION OF THE DRAWINGS

FIG. 1A-FIG. 1G. CD63 is upregulated in lung adenocarcinomas on the protein and the gene expression level. (FIG. 2A) Schema of CD63. (FIG. 3B) Representative IHC stains against CD9, CD63, CD81, and CD51 on tissue sections. Scale bar=200 μm. (FIG. 4C) Quantification of positive samples for CD9, CD63, CD81, and CD151 expression. (FIG. 5D) Representative ISH stains in epithelial cells with different intensities against CD63. (FIG. 6E) Quantification of CD63 gene expression in epithelial cells in lung cancer and matched normal lung tissue. Every row represents an individual sample. Wilcoxon-Test. p<0.001. n=45. (FIG. 7F) Representative ISH stains in stroma with negative and positive gene expression of CD63. (FIG. 8G) Quantification of CD63 gene expression in stroma in lung cancer and matched normal lung tissue. Every row represents an individual sample. Wilcoxon-Test. p<0.001. n=45. (FIG. 9H) Kaplan-Meier-Plot for CD63 expression in lung adenocarcinoma.

FIG. 2A-FIG. 2H. CD63 is transferred from fibroblasts to cancer cells via syncyctin1. (FIG. 2A) Representative IF stains against CD63 and FSP1 in normal lung fibroblasts. (NFB) and cancer-associated fibroblasts (CAF). Yellow bar graph=25 μm. (FIG. 2B) Western Blot analysis of lung fibroblasts samples and lung cancer cell lines for the expression of CD9, CD63, CD81, and CD151. (FIG. 2C) CD63 secretion into the supernatant by lung cancer cells and lung fibroblasts. The medium alone was supplemented serum-free DMEM/F12. Tukey's multiple comparisons test. ***p<0.001. n=4. (FIG. 2D) FACS histogram for CD63 of A549 cells or after 24 hours co-incubation with lung cancer fibroblasts. Corresponding quantification. A549 cells alone served as the reference values. Two-sided student's t-test. ***p<0.001. n=4. (FIG. 2E) Representative FACS plots of unlabeled A549 cells alone or after 24 hours of co-incubation with lung fibroblasts expressing GFP-labeled CD63. Corresponding quantification of GFP+ cells. Two-sided student's t-test. ***p<0.001. n=3. (FIG. 2F) Representative FACS plots of unlabeled A549 cells cultured in regular serum-free medium or with the supernatant of GFP-labeled CD63 expressing fibroblasts. Corresponding quantification for GFP+ cells. Two-sided student's t-est. ***p<0.005. n=3. (FIG. 2G) FACS histogram for CD63 of LC19 cells or after 24 hours co-incubation with lung cancer fibroblasts. (FIG. 2H) Western Blot for Syncytin1 in LC19 and A549 control cells or after CD63 deletion.

FIG. 3A-FIG. 3K. CD63 regulates phagocytosis in vitro and in vivo. (FIG. 3A) Histogram plots for CD63 expression in LC19, A549, and H810 cells without (Ctrl) and with CD63 ko. (FIG. 3B) Schema for the in vitro phagocytosis assays. (FIG. 3C) Representative FACS plots of the phagocytosis assays with LC19, A549, and H810 cells. Shown are the CD11b+ macrophage gates. GFP positivity as a sign for the uptake of GFP labeled target cells. (FIG. 3D) Corresponding quantification of the phagocytosis assays. The highest individual phagocytosis value represents 1. Two-sided student's t-test. ***p<0.001. n=4-7. (E) Histogram for CD63 in IPF fibroblasts (IPF) with and without CD63 knockout. (FIG. 3F) Representative FACS plots of the phagocytosis assays with IPF and CAF. Shown are the CD11b+ macrophage gates. GFP positivity as a sign for the uptake of GFP labeled target cells. ipf-fibroblasts. (FIG. 3G) Corresponding quantification. The highest individual phagocytosis value represents 1. Two-sided student's t-test. ***p<0.001. n=4. (FIG. 3H) Relative phagocytosis values after CD63 knockout correlated with wild-type CD63 expression in flow cytometry. (FIG. 3I) Schema for the in vivo phagocytosis measurement. (FIG. 3J) Representative FACS plots of LC19, A549, and H810 cells without (Ctrl) and with CD63 depletion (KO). Shown are CD11b+ macrophage gates (left) and the GFP+ cancer cell gate (right). (FIG. 3K) Corresponding quantification of the phagocytosis index (Percentage of phagocytosing macrophages+percentage of phagocytosed cancer cells). The highest individual value for each cancer cell line represents 1. Two-sided student's t-test. *p<0.05 ***p<0.001. n=4-7.

FIG. 4A-FIG. 4J. CD63 on cancer cells interacts with CD9 on macrophages. (FIG. 4A) IF images from a proximity ligation assay of GFP labeled LC19 cells. (FIG. 4B) Corresponding quantification of CD63/CD9 proximity via measuring MFI. Tukey's test for multiple comparisons. ***p<0.001. n=6. (FIG. 4C) High power view showing CD63/CD9 proximity on the surface on LC19 cells (LC19) but not on the surface LC19 cells after CD63 knockout. (FIG. 4D) Representative IHC stains against CD64. Box=Shown in higher magnification below. Scale bar=200 μm. (FIG. 4E) Quantification of CD64 cells on tissue cores in lung cancer sections with none/weak CD63 expression, intermediate CD63 expression, or strong CD63 expression according to the CD63 gene expression quantification in FIG. 2. Tukey's test for multiple comparisons. *p<0.05. n=14-18. (FIG. 4F) Kaplan-Meier-Plot for CD64 gene expression in lung adenocarcinomas. (FIG. 4G) Spearman correlation between CD63 and CD14, CD64, and CD68. (FIG. 4H) Schema of a macrophage differentiation assay. Bead-purified CD14+ monocytes were added to lung cancer cells, either with or without CD63 KO, and incubated for 12 days before measuring M1 and M2 differentiation markers with flow cytometry. (FIG. 4I) Representative histogram for CD86 expression. (FIG. 4J) Quantification of different M1 (HLA-DR, CD11c, and CD86) and M2 markers (CD163, CD204, and CD206). Sidak's test for multiple comparisons. ***p<0.001. n=3.

FIG. 5A-FIG. 5L. CD63 affects cancer stem cell-associated features in vitro and in vivo. (FIG. 5A) EdU proliferation assay of LC19 cells either without (LC19) or with a CD63 knockout (ko). Cells were harvested 45 and 90 minutes after adding EdU. MFI was measured in the AF647 channel. Sidak's multiple comparisons test. ***p<0.001. n=4. (FIG. 5B) EdU proliferation assay of A549 cells either without (A549) or with a CD63 knockout (ko). Cells were harvested 45 and 90 minutes after adding EdU. MFI was measured in the AF594 channel. Sidak's multiple comparisons test. ***p<0.001. n=4. (FIG. 5C) Western Blot for several CSC-associated markers and P14K. (FIG. 5D) Optical imaging 3 and 13 days after transplantation of LC19 cells without (Ctrl) or with a CD63 knockout (ko) under the kidney capsule of NSG mice. (FIG. 5E) Corresponding quantification of photon emissions. Sidak's multiple comparisons test. **p<0.01. n=10. (FIG. 5F) Representative visualization of GFP+ cells under the dissection microscope and IHC stains against GFP. *=Tumor. Red scale bar=1000 μm. Black scale bar=200 μm. (FIG. 5G) Optical imaging 3 and 13 days after transplantation of A549 cells without (Ctrl) or with a CD63 knockout (ko) under the kidney capsule of NSG mice. (FIG. 5H) Corresponding quantification of photon emissions. Sidak's multiple comparisons test. **p<0.01. n=8. (FIG. 5I) Representative visualization of GFP+ cells under the dissection microscope and HE stains. GFP. *=Tumor. Red scale bar=1000 μm. Black scale bar=200 μm. (FIG. 5J) Optical imaging 2 and 64 days after transplantation of H810 cells without (Ctrl) or with a CD63 knockout (ko) under the kidney capsule of NSG mice. (FIG. 5K) Corresponding quantification of photon emissions. Sidak's multiple comparisons test. **p<0.01. n=8-10. (FIG. 5L) Representative HE stains of the tumor grafted under the kidney capsule and of the liver. GFP. *=Tumor. Scale bar=200 μm.

FIG. 6A-FIG. 6C. CD63 is upregulated in mouse lung tumors. (FIG. 6A) Schema of lung tumor model. (FIG. 6B) HE sections of lung adenomas. Box=Adenoma shown in higher magnification on the right. *Additional adenomas. Scale bar=200 μm. (FIG. 6C) IF stains against CD63 and EpCam on normal bronchoepithelium and adenoma.

FIG. 7A-FIG. 7C. Kaplan-Meier Curve for CD63 in different stages of lung adenocarcinomas. (FIG. 7A) Stage I. (FIG. 7B) Stage II. (FIG. 7C) Stage Ill.

FIG. 8A-FIG. 8B. Characteristics of LC19 cells. (FIG. 8A) FACS histogram for CD326 (Epcam) expression in LC19 cells and fibroblasts. (FIG. 8B) Light microscopy of LC19 cell cultures with different magnifications, showing alveolae-shaped cell formations.

FIG. 9A-FIG. 9B. Transplantation of LC19 cells under the renal capsule of NSG mice. (FIG. 9A) Images taken with a dissection microscope of ectopic tumors of LC cells without a CD63 knockout (LC19) and with a CD63 knockout (ko). Scale bar=2 mm. (FIG. 9B) Quantification of kidneys depicting surviving GFP+LC19 cells. Mann-Whitney-Test. **p<0.01. n=8.

FIG. 10A-FIG. 10B. Transplantation of A549 under the renal capsule of NSG mice. (FIG. 10A) Images taken with a dissection microscope of ectopic tumors of A549 cells without a CD63 knockout (A549) and with a CD63 knockout (ko). Scale bar=1 mm. (FIG. 10B) Quantification of kidneys depicting surviving GFP+A549 cells. Mann-Whitney-Test. **p<0.01. n=8.

FIG. 11A-FIG. 11D. Subcutaneous tumor model of LC19 cells. (FIG. 11A) Experimental outline. (FIG. 11 B) Tumor volume measurements starting on day 14 of tumors without (LC19) and with (ko) CD63 knockout. Sidak's test for multiple comparisons. *p<0.05 **p<0.01 ***p<0.001. n=8. (FIG. 11C) Survival curve until tumors were reaching the maximal volume (1250 mm3). Mantel-Cox test. **p<0.01. n=8. (FIG. 11D) Representative H&E stains and IF stains against Ki67 of tumors.

FIG. 12A-FIG. 12B. Overview of CD63 expression in healthy tissues across cell types (FIG. 12A) and core tissues (FIG. 12B).

FIG. 13A-FIG. 13B. Four snRNA-seq methods were applied to 25 samples from 16 donors to generate a cross-tissue atlas of 209,126 nuclei profiles and (FIG. 13A) map the expression of CD63 in human healthy tissues labeled by dark dots. These data confirm that CD63 expression is rare in healthy human tissues and mainly present in a minor subset of myofibroblasts, myocytes and epithelial cells. (FIG. 13B) cell type mapping.

FIG. 14A-FIG. 14C. (FIG. 14A) Meta-analysis of scRNA seq data of healthy lung tissue (GSE130148) reveal no significant expression of CD63 in healthy lung except low expression in macrophages. (FIG. 14B-C) UMAP, heatmap and gene expression analysis depicting single cell data according to cell type as scatter plot, all cells are shown in the color scale as % of maximal expression.

FIG. 15A-FIG. 15C. (FIG. 15A) Meta-analysis of scRNA seq of human healthy skin demonstrates CD63 expression to be high in fibroblasts followed by endothelial cells. (FIG. 15B) UMAP and (FIG. 15C) heatmap gene expression analysis of GSE130973 demonstrate scatter plots colored according to cell type group, in addition all cells are shown on the color scale as % of maximal expression.

FIG. 16. Three data sets, 69 cell lines, 52 human tissues and 18 blood cell types and peripheral blood mononuclear cells (PBMC) have been analyzed by RNA-seq to estimate the transcript abundance of each protein-coding gene. The nTPM number quantifies CD63 expression in immune cells across these data sets.

FIG. 17. ATAC and ChIP-seq results demonstrate increased chromatin accessibility of the promoter regions of CD63 in lung fibroblasts derived from lung fibrosis patients (#1,3). Chromatin accessibility of the CD63 promoter was reduced (#2,4) when JUN was knocked down with CRISPR-Cas9 suggesting that JUN regulates promoter accessibility of CD63 in lung fibroblasts in lung fibrosis.

FIG. 18A-FIG. 18C. Analysis of 114,000 cells from 20 fibrotic lungs and 10 control lungs identified 31 distinct cell types and revealed CD63 expression in macrophage and epithelial cells GSE135893. (FIG. 18A-B) Cell distribution in ILD and Control (interstitial lung disease/lung fibrosis=ILD, control=non-fibrosis healthy lung). (FIG. 18C) CD63 expression in the lung is quantified as % maximum expression across cell types.

FIG. 19A-FIG. 19J. Single-cell atlas of human chronic kidney disease (CKD) due to hypertensive nephrosclerosis and mouse UUO-induced fibrosis model. (FIG. 19A) UMAP embedding of 51,849 CD10-single cells from 15 human kidneys and CD63 (FIG. 19B) is mainly expressed in monocytes, fibroblasts, proximal tubule cells and pericytes. TIMP1 (FIG. 19C) is mainly expressed in fibroblasts and macrophages. (FIG. 19D) CD63 is significantly overexpressed in monocytes, fibroblasts, descending thin limb and pericytes in CKD. (FIG. 19E) TIMP1 is significantly overexpressed in fibroblasts in CKD. (FIG. 19F) UMAP embedding of 7,245 PDGFRα+PDGFRβ+ cells in mouse kidney and CD63 (FIG. 19G) is expressed in all cell types. TIMP1 (FIG. 19H) is mainly expressed in myofibroblasts. (FIG. 19I) CD63 is significantly overexpressed in mesenchymal cells and fibroblasts in UUO model. (FIG. 19J) TIMP1 is significantly overexpressed in every cell type detected in UUO model.

FIG. 20A-FIG. 20D. Expression of CD63 and fibrotic proteins in IPF tissues (FIG. 20A) Immunofluorescently labeled CD63 in fresh frozen lung fibrosis (IPF) tissue. (FIG. 20B) and (FIG. 20C) Immunofluorescently labeled c-kit and TIMP1 in FFPE IPF tissue. C-kit and TIMP1 are known as associated proteins of CD63. (FIG. 20D) Immunofluorescently labeled αSMA and Col I in FFPE IPF tissue. Magnification, 40×, 60×.

FIG. 21A-FIG. 21C. Activation of TGFβ1 signaling pathway elevated the expression of fibrotic related proteins in 3 kidney cell lines. (FIG. 21 FIG. 21A) Human epithelial (HK2), (FIG. 21 B) rat epithelial (NRK52E), (FIG. 21C) rat fibroblast (NRK49F) cell lines were treated with TGFβ1 (10 ng/ml) or TGFβ1 (10 ng/ml) plus LY2109761 (2.5 μM) for indicated times in hours. RT-qPCR analysis showed the elevated expression of fibrotic related proteins. CD63 and its known interactor TIMP1 were not increased in any of the cell lines, indicated that CD63 did not involve in TGFβ1 signaling pathway.

FIG. 22A-FIG. 22E. (FIG. 22A) CD63 expression correlates with poor survival in cancer patients and is one of the most significantly overexpressed immune proteins in human patients with liver diseases. Using patient single cell transcriptomic data from 25 patients (>50,000 cells each) we compared the single cell populations identified between the healthy and diseased population. Using hepatocellular carcinoma as our disease model we clustered multiple populations of cell specific subtypes in both healthy and diseased liver. (FIG. 22B) Cell specific populations in HCC cluster differently than cellular subpopulations in healthy livers. (FIG. 22C) Of the cell specific clusters, clusters 5, 8, 21 and 25 are pathogenic fibroblast subclusters. Cluster 3 comprises healthy fibroblasts. (FIG. 22D) Re-clustering the selected fibroblast subclusters demonstrates clusters 2, 8 and 5 as highly pathogenic fibroblast clusters in liver cancer whereas clusters 1 and 0 are highly enriched fibroblasts in healthy livers. (FIG. 22E) UMAP indicating cell population clusters highly enriched in liver cancer and cell population clusters highly enriched in normal livers. We further perturbed the individual proteins that are significantly enriched in the pathogenic clusters and the healthy clusters.

FIG. 23A-FIG. 23D. CD63 is significantly upregulated in primary human liver tissue in HCC. (FIG. 23A) To validate the highest gene enrichment obtained in cell population clusters highly enriched in liver cancer and cell population clusters highly enriched in normal livers, we demonstrate a UMAP indicating CD63 expression is the highest in the pathogenic fibroblast clusters in liver cancer. This enrichment is significantly higher than that obtained in normal healthy liver clusters. (FIG. 23B) To identify the percent cell enrichment in each cluster, we analyzed the frequency of cells observed in the most pathogenic cluster (cluster 5). We demonstrate high enrichment of pathogenic fibroblasts that are CD63 positive (˜800 cells) relative to CD63+ve cells in the normal liver (˜200 cells). After isolating primary fibroblasts from liver cancer patients, CD63 staining of primary cells reveals significant overexpression of CD63 in the primary patient fibroblasts. (FIG. 23C) Flow cytometry validation further demonstrated this point with CD63 expression (Mean Fluorescence Intensity >2-fold higher in HCC samples compared to healthy control cells). (FIG. 23D) Comparing across the most highly expressed genes in HCC, we demonstrate CD63 as one of the most significantly enriched genes in liver cancer patients with expression levels comparable to known molecular targets of cancer like LGALS1, CD47 and CXCR3.

FIG. 24A-FIG. 24D. CD63 is significantly enriched in other liver diseases like NASH, NAFLD and liver cirrhosis. (FIG. 24A) To analyze the expression of CD63 in other liver pathologies like NASH, NAFLD and Liver cirrhosis, we stained NASH human tissue for CD63 expression. CD63 is highly expressed in NASH human tissue compared to healthy liver tissue. (FIG. 24B) Flow cytometry analyses further demonstrated that CD63 expression was significantly higher in NASH patient primary cells relative to normal liver cells. (FIG. 24C) To further evaluate CD63 expression both in NAFLD and cirrhotic liver, we used RNA scope to analyze CD63 expression which can be determined from probe expression. CD63 expression is high in NAFLD and cirrhotic liver. This expression co-localized with FSP1 which is a known marker of fibrosis. These data describe the expression levels of CD63 which exhibit patterns similar to that of fibrotic liver markers. (FIG. 24D) Finally, to evaluate the role of CD63 in the development of liver pathogenic diseases like liver cirrhosis, we used flow cytometric analyses to demonstrate that in fatty liver patient primary cells there is a population of cells that is CD63 positive but a large population of cells that are CD63 negative. However, looking at primary cells from liver cirrhosis patients, it is evident that a majority of the cell populations highly express CD63. These results strongly suggest that CD63 could play an important role in the fatty liver progressing from a normal phenotype to a more aggressive pathogenic fibrotic phenotype that is observed in liver cirrhosis patients.

FIG. 25. Genetic knock-out of CD63 increases phagocytosis of pathogenic liver fibroblasts. Genetically knocking out CD63 in pathogenic human fibroblasts increases the number of human macrophages when tested in a co-culture system staining for M2 macrophage marker CD204. Since phagocytosis is a prime mechanism through which several immune therapeutic antibodies target pathogenic diseases for treatment, we believe blocking CD63 is an essential treatment mechanism through which we can elicit an immune response against pathogenic fibroblasts in diseases like liver cirrhosis, NASH, HCC and NAFLD paired student t-test (p<0.001).

FIG. 26A-FIG. 26P. CD63 knockout increases phagocytosis in vitro. (FIG. 26A) IF stains against CD63 in LC19 cells without (Ctrl) and with CD63 ko. Scale bar=25 μm. (FIG. 26B) Histogram for CD63 expression in LC19 cells without (Ctrl) and with CD63 ko. (FIG. 26C) Representative FACS plots and corresponding quantification. Shown are the CD11b+ macrophage gates. GFP positivity as a sign for the uptake of GFP labeled LC19 cells. The highest individual phagocytosis value represents 1. Two-sided student's t-test. ***p<0.001. n=7. (FIG. 26D) IF stains against CD63 in A549 cells without (Ctrl) and with CD63 ko. White scale bar=25 μm. Red scale bar=10 μm. (FIG. 26E) Histogram for CD63 expression in A cells without (Ctrl) and with CD63 ko. (FIG. 26F) Representative FACS plots and corresponding quantification. Shown are the CD11b+ macrophage gates. GFP positivity as a sign for the uptake of GFP labeled A549 cells. The highest individual phagocytosis value represents 1. n=5. (FIG. 26G) Histogram for CD63 in ipf fibroblasts with and without CD63 knockout. (FIG. 26H) Representative FACS plots and corresponding quantification. Shown are the CD11b+ macrophage gates. GFP positivity as a sign for the uptake of GFP labeled ipf-fibroblasts. The highest individual phagocytosis value represents 1. Two-sided student's t-test. ***p<0.001. n=4. (FIG. 26I) Histogram for CD63 in luc-fibroblasts with and without CD63 knockout. (FIG. 26J) Representative FACS plots and corresponding quantification. Shown are the CD11b+ macrophage gates. GFP positivity as a sign for the uptake of GFP labeled luc-fibroblasts. The highest individual phagocytosis value represents 1. Two-sided student's t-test. **p<0.01. n=4. (FIG. 26K) Relative phagocytosis values after CD63 knockout plotted against the wild-type CD63 expression in flow cytometry. (FIG. 26L) Representative phagocytosis FACS plots of luc-fibroblasts either without any treatment (untreated) or after antibody-mediated CD63 blockade (anti-CD63). Shown are the CD11b+ macrophage gates. GFP positivity as a sign for the uptake of GFP labeled luc-fibroblasts. (FIG. 26M) Corresponding quantification. The highest phagocytosis value represents 1. Untreated=No treatment. αCD9=CD9 blockade on macrophages. αCD63+CD9=CD63 blockade on fibroblasts and CD9 blockade on macrophages. αCD63=CD63 blockade on fibroblasts. Tukey's test for multiple comparisons. **p<0.01 ***p<0.001. n=4. (FIG. 26N) IF images from a proximity ligation ligation assay of GFP labeled LC19 cells. (FIG. 26O) Corresponding quantification of CD63/CD9 proximity via measuring MF. Tukey's test for multiple comparisons. ***p<0.001. n=6. (FIG. 26P) High power view showing CD63/CD9 proximity on the surface on LC19 cells (Ctrl) but not on the surface LC19 cells after CD63 knockout.

FIG. 27A-FIG. 27D. FIG. 27A) Increased CD63 expression in MG63 and 143b human osteosarcoma cells shown by immunofluorescence and (FIG. 27B) flow cytometry. (FIG. 27C) Increased phagocytic removal of MG63 and 143b human osteosarcoma cells after knockout of CD63 with CRISPR Cas 9. Tukey's test for multiple comparisons. ***p<0.001.

FIG. 28. (FIG. 28A, C) Engraftment of human osteosarkoma cells 143b and MG63 in immune compromised mice under the kidney capsule and (FIG. 28B, D) and live imaging of tumor engraftment with luciferase over 15 days with and without the deletion of CD63 as indicated demonstrate significant reduction of tumor growth for MG63 when CD63 was deleted. (FIG. 28E-G) Histopathologic sections stained with H&E demonstrate significant reduction in tumor growth and replacement of tumor cells with inflammatory infiltrate after CD63 knockout. (FIG. 28H) Increased CD63 expression in ˜20% of human soft tissue sarcomas (41 human sarcoma samples analyzed).

FIG. 29A-FIG. 29C. (FIG. 29A-C) Increased CD63 expression in lung fibroblasts and lung epithelial cells in JUN inducible mouse model of lung fibrosis by flow cytometry and immunostaining. Student's t-test ***p<0.001.

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

### Definitions

It is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

The term “antibody” herein is used in the broadest sense and specifically covers monoclonal antibodies, polyclonal antibodies, monomers, dimers, multimers, heavy chain only antibodies, three chain antibodies, single chain Fv, single domain antibodies, NANOBODIES®, etc., and also include antibody fragments with or without pegylation, so long as they exhibit the desired biological activity (Miller et al (2003) Jour. of Immunology 170:4854-4861).

The term antibody may reference a full-length heavy chain, a full length light chain, an intact immunoglobulin molecule including a functional Fc sequence; or an immunologically active portion of any of these polypeptides, i.e., a polypeptide that comprises an antigen binding site that immunospecifically binds an antigen of a target of interest or part thereof.

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; nanobodies or domain antibodies comprising single Ig domains from human or non-human species or other specific single-domain binding modules including non-antibody binding proteins such as, but not limited to, adnectins and anticalins; and multispecific or multivalent structures formed from antibody fragments.

The term “NANOBODY®” as used herein refers to a single domain antibody consisting of a single monomeric variable domain (also referred to as a variable heavy homodimer [VHH] domain or immunoglobulin single variable domains or ISVs). The single domain antibodies are naturally produced by animals belonging to the camelid family. Nanobodies are smaller than human antibodies, where ISV are generally 12-15 kDa, human antibodies are generally 150-160 kDa, Fab fragments are ˜50 kDa and single-chain variable fragments are ˜25 kDa. NANOBODIES® provide specific advantages over traditional antibodies including smaller sizes, they are more easily engineered, higher chemical and thermo stability, better solubility, deeper tissue penetration, the ability to bind small cavities and difficult to access epitopes of target proteins, the ability to manufacture in microbial cells (i.e. cheaper production costs relative to animal immunization), and the like.

Immunoglobulin sequences, such as antibodies and antigen binding fragments derived therefrom (e.g. ISVs) are used to specifically target the respective antigens disclosed herein. The generation of immunoglobulin single variable domains such as e.g., VHHs or ISV may involve selection from phage display or yeast display, for example ISV can be selected by utilizing surface display platforms where the cell or phage surface display a synthetic library of ISV, in the presence of tagged antigen. A fluorescent secondary antibody directed to the tagged antigen is added to the solution thereby labeling cells bound to antigen. Cells are then sorted using any cell sorting platform of interest e.g., magnetic-activated cell sorting (MACS) or fluorescence-activated cell sorting (FACS). Sorted clones are amplified, resulting in an enriched library of clones expressing ISV that bind antigen. The enriched library is then re-screened with antigen to further enrich for surface displayed antigen binding ISV. These clones can then be sequenced to identify the sequences of the ISV of interest and further transferred to other heterologous systems for large scale protein production. Yeast surface display has been successfully used to generate specific ISVs as shown in McMahon et al. (2018) Nature Structural Molecular Biology 25(3): 289-296 which is specifically incorporated herein by reference.

Alternatively, similar immunoglobulin single variable domains can be generated and selected by the immunization of an experimental animal such as a llama, construction of phage libraries from immune tissue, and

Unless indicated otherwise, the term “immunoglobulin single variable domain” or “ISV” is used as a general term to include but not limited to antigen-binding domains or fragments such as VHH domains or VH or VL domains, respectively. The terms antigen-binding molecules or antigen-binding protein are used interchangeably and include also the term NANOBODIES®. The immunoglobulin single variable domains can be light chain variable domain sequences [e.g., a VL-sequence), or heavy chain variable domain sequences (e.g., a VH-sequence); more specifically, they can be heavy chain variable domain sequences that are derived from a conventional four-chain antibody or heavy chain variable domain sequences that are derived from a heavy chain antibody. Accordingly, the immunoglobulin single variable domains can be single domain antibodies, or immunoglobulin sequences that are suitable for use as single domain antibodies, “dAbs”, or immunoglobulin sequences that are suitable for use as dAbs, or NANOBODIES®, including but not limited to VHH sequences. An amino acid sequence such as e.g. an immunoglobulin single variable domain or polypeptide is said to be a “VHH1 type immunoglobulin single variable domain” or “VHH type 1 sequence”, if said VHH1 type immunoglobulin single variable domain or VHH type 1 sequence has 85% identity (using the VHH1 consensus sequence as the query sequence and use the blast algorithm with standard setting, i.e., blosom62 scoring matrix) to the VHH1 consensus sequence and mandatorily has a cysteine in position 50, i.e., C50 (using Kabat numbering). See, for example, VHH domains from Camelids in the article of Riechmann and Muyldermans, J. Immunol. Methods 2000 Jun. 23; 240 (1-2): 185-195.

The invention includes immunoglobulin sequences of different origin, comprising mouse, rat, rabbit, donkey, human and camelid immunoglobulin sequences. The immunoglobulin single variable domain includes fully human, humanized, otherwise sequence optimized or chimeric immunoglobulin sequences. An immunoglobulin variable domain and structure of an immunoglobulin single variable domain can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively.

Such immunoglobulin single variable domains may be derived in any suitable manner and from any suitable source, and may for example be naturally occurring VHH sequences (i.e., from a suitable species of Camelid, e.g., llama) or synthetic or semi-synthetic VHs or VLs (e.g., from human). Such immunoglobulin single variable domains may include “humanized” or otherwise “sequence optimized” VHHs, “camelized” immunoglobulin sequences (and in particular camelized heavy chain variable domain sequences, i.e., camelized VHs), as well as human VHs, human VLs, camelid VHHs that have been altered by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein.

CD63 antigen is a member of the transmembrane 4 superfamily, also known as the tetraspanin family. Most of these members are cell-surface proteins that are characterized by the presence of four hydrophobic domains. The proteins mediate signal transduction events that play a role in the regulation of cell development, activation, growth, and motility. This encoded protein is a cell surface glycoprotein that is known to complex with integrins. It may function as a blood platelet activation marker. Deficiency of this protein is associated with Hermansky-Pudlak Syndrome. Also this gene has been associated with tumor progression. The use of alternate polyadenylation sites has been found for this gene. Alternative splicing results in multiple transcript variants encoding different proteins. CD63 is extensively and variably glycosylated and the EC2 region contains three potential N-linked glycosylation sites (N130, N150, and N172). An association between CD63Wt and β2 integrin (CD18) was shown by co-internalisation of these proteins. Interactions with CD63 may therefore affect the trafficking and function of β2 integrins. In addition, CD63 has been reported to interact with CD117 and CD82. Reference sequences of CD63 include, for example, NP_001244318; NP_001244319; NP_001244320; NP_001244321; NP_001244329.

Anti-CD63 agent. As used herein, the term “anti-CD63 agent” or “agent that provides for CD63 blockade” refers to any agent that reduces the binding of CD63 (e.g., on a target cell) to a counter receptor on a phagocytic cell, and facilitates the preferential phagocytosis of target cells expressing CD63. Non-limiting examples of suitable anti-CD63 reagents include soluble CD63 polypeptides, anti-CD63 antibodies or antibody fragments, small molecules, etc.

In an exemplary assay, target cells are incubated in the presence or absence of the candidate agent and in the presence of an effector cell, e.g. a macrophage or other phagocytic cell. An agent for use in the methods of the invention will up-regulate phagocytosis by at least 5% (e.g., at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 500%, at least 1000%) compared to phagocytosis in the absence of the agent.

Anti-CD63 antibodies. In some embodiments, a subject anti-CD63 agent is an antibody that specifically binds CD63 (i.e., an anti-CD63 antibody) and reduces the interaction between CD63 on one cell (e.g., an infected cell) and a counter receptor on another cell (e.g., a phagocytic cell). A suitable anti-CD63 antibody that is an “anti-CD63 agent” can be referred to as a “CD63-blocking antibody”. Suitable anti-CD63 antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof. In some embodiments an anti-CD63 antibody comprises a human IgG Fc region, e.g. an IgG1, IgG2a, IgG2b, IgG3, IgG4 constant region.

Anti-CD63 antibodies publicly known and available from a number of suppliers. This target gene encodes the protein ‘CD63 molecule’ in humans and may also be known as LAMP-3, ME491, MLA1, OMA81 H, CD63 antigen, and CD63 antigen (melanoma 1 antigen). Examples of commercially available antibodies include, without limitation, H5C6 (BioLegend); MAB5048 (R&D Systems); H5C6 (Novus Biologicals); MEM-259 (BioRad); Ts63 (AbCam); 12-0639-42 (Thermo Fisher Scientific), 8A12 (Cosmo Bio), etc.

Anti-CD63 antibodies may be generated in the form of ISVs. Suitable ISVs may be generated using the antigen binds domains of other antibodies directed towards CD63. For instance, an ISV may be generated using the antigen binding domain of the anti-CD63 antibody disclosed in Goeij et al. Mol Cancer Ther. 2016 November; 15(11):2688-2697, herein specifically incorporated by reference.

Soluble CD63 polypeptides. In some embodiments, a subject anti-CD63 agent is a soluble CD63 polypeptide that acts as a competitive inhibitor. A suitable soluble CD63 polypeptide can bind counter receptors without activating or stimulating signaling that would inhibit phagocytosis. Instead, suitable soluble CD63 polypeptides facilitate the preferential phagocytosis of target cells. Those cells that express higher levels of CD63 (e.g., tumor cells) relative to normal, non-target cells (normal cells) will be preferentially phagocytosed. In some cases, a suitable soluble CD63 polypeptide can be a fusion protein. Suitable soluble CD63 polypeptides also include any peptide or peptide fragment comprising variant or naturally existing CD63 sequences (e.g., extracellular domain sequences or extracellular domain variants) that can block without inhibiting phagocytosis.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer and/or fibrosis, including without limitation, tumor fibrosis, cardiac fibrosis, liver fibrosis, kidney fibrosis, lung fibrosis, dermal scarring and keloids, Alzheimer's disease, etc. Subjects may be human, but also include other mammals, particularly those mammals useful as laboratory models for human disease, e.g. mouse, rat, etc.

Fibrosis is the formation of excess connective tissue causing stromal hardening and scar formation. Fibroblasts are connective-tissue cells of mesenchymal origin. They are stromal cells which control tissue integrity. Fibroblasts maintain ECM homeostasis through both deposition of ECM and secretion of matrix metalloproteinases (MMPs) to remodel the ECM. Fibroblasts also regulate adjacent epithelial cells directing epithelial proliferation and differentiation.

Fibroblasts are considered the main effectors of fibrosis in both normal and pathologic settings. During inflammation, fibroblasts become “activated” and are referred to as myofibroblasts which are the main collagen producers in the body. Fibroblasts associated with normal wound healing are phenotypically distinct from fibroblasts associated with cancer; fibroblasts within the TME are referred to as cancer-associated fibroblasts (CAFs) and they have a unique expression profile and function which significantly contributes to cancer-related fibrosis. In contrast to normal fibroblasts, CAFs have increased autocrine signaling ability and proliferation tendencies.

Exemplary forms of fibrosis include, but are not limited to, tumor fibrosis, cardiac fibrosis, liver fibrosis such as liver cirrhosis, kidney and bladder fibrosis, lung fibrosis, dermal scarring and keloids, wound healing and adhesions, post-irradiation fibrosis, fibrosis related to chronic graft v host disease (GvHD), and Alzheimer's disease. In still further embodiments, cardiac fibrosis is associated with hypertension, hypertensive heart disease (HHD), myocardial infarction (MI), cardiac scarring related to ischemia congestive heart failure, cardiomyopathy, post-myocardial infarction defects in heart function, atherosclerosis, and restenosis. Kidney fibrosis may include, but not be limited to, diabetic nephropathy, vesicoureteral reflux, tubulointerstitial renal fibrosis, glomerulonephritis or glomerular nephritis (GN), focal segmental glomerulosclerosis, membranous glomerulonephritis, or mesangiocapillary GN. Liver fibrosis may include, but not be limited to, cirrhosis, and associated conditions such as chronic viral hepatitis, non-alcoholic fatty liver disease (NAFLD), alcoholic steatohepatitis (ASH), non-alcoholic steatohepatitis (NASH), primary biliary cirrhosis (PBC), biliary cirrhosis, autoimmune hepatitis). Lung fibrosis may include idiopathic pulmonary fibrosis (IPF) or cryptogenic fibrosing alveolitis, chronic fibrosing interstitial pneumonia, interstitial lung disease (ILD), and diffuse parenchymal lung disease (DPLD)), lung scarring including without limitation damage from bacterial viral or fungal infection, emphysema, chronic obstructive pulmonary disease (COPD); and chronic asthma may also be prevented, treated, or ameliorated with compositions of described herein. Also included is fibrosis of the eye and lens, for example glaucoma; age-related macular degeneration (wet AMD and dry AMD), fibrosis of the lens, periorbital fibrosis as in IgG4-related disease, hyperthyroidism, etc. Uterine fibroids are also if interest for treatment.

One example of fibrosis of the lung is idiopathic pulmonary fibrosis (IPF), the most common form of idiopathic interstitial pneumonia, causes progressive pulmonary fibrosis. Symptoms and signs develop over months to years and include exertional dyspnea, cough, and fine (Velcro) crackles. Diagnosis is based on history, physical examination, high-resolution CT, and/or lung biopsy, if necessary. Treatment may include antifibrotic drugs and oxygen therapy. Most patients deteriorate; median survival is about 3 years from diagnosis.

Idiopathic pulmonary fibrosis, identified histologically as usual interstitial pneumonia, accounts for most cases of idiopathic interstitial pneumonia. IPF affects men and women >50 in a ratio of 2:1, with a markedly increased incidence with each decade of age. Current or former cigarette smoking is most strongly associated with the disorder. There is some genetic predisposition; familial clustering occurs in up to 20% of cases.

The key histologic findings of idiopathic pulmonary fibrosis are subpleural fibrosis with sites of fibroblast proliferation (fibroblast foci) and dense scarring, alternating with areas of normal lung tissue (heterogeneity). Scattered interstitial inflammation occurs with lymphocyte, plasma cell, and histiocyte infiltration. Cystic abnormality (honeycombing) occurs in all patients and increases with advanced disease. A similar histologic pattern uncommonly occurs in cases of interstitial lung diseases of known etiology.

A variety of drugs have been tried in various fibroses, particularly lung fibrosis, with very little success. Anti-inflammatory drugs including prednisolone and azathioprine have little effect on fibrosis suggesting that inflammation is only the initiator, but not the driver of the disease. The use of non-specific anti-proliferatives like colchicine and cyclophosphamide will also prevent repair of the fibrotic tissue by impairing e.g. epithelial growth. Treatment with IFN-γ has shown some utility but is limited by severe side effects.

By the time a typical patient presents with fibrosis-related symptoms (e.g. difficulty breathing for lung fibrosis, cirrhosis for liver fibrosis, etc.), the fibrosis in the target organ is often quite severe, with much of the target organ architecture having been replaced with extracellular matrix. Stopping this ongoing fibrosis can extend lifespan and improve quality of life. Areas of the target organ where the fibrosis is not extensive may be restored to normal architecture with suitable treatment.

In some embodiments, tumor fibrosis is associated with pancreatic cancer. Pancreatic cancer is characterized by a prominent desmoplastic/stromal reaction. Pancreatic stellate cells (PSCs) are the principal source of fibrosis in the stroma and interact closely with cancer cells to create a tumor facilitatory environment that stimulates local tumor growth and distant metastasis. Pancreatic fibrosis is initiated when PSCs become activated and undergo morphological and functional changes, so that the rate of extracellular matrix (ECM) deposition exceeds the rate of ECM degradation in the gland. It is now well established that pancreatic cancer cells activate PSCs leading to increased fibrosis. There is significant evidence showing that the intense stromal/desmoplastic reaction around tumor elements (a feature of the majority of pancreatic cancers) plays an important role in tumor progression. A key histopathological feature of pancreatic cancer which is associated with its innate clinical and biological aggressiveness is its desmoplastic (stromal) reaction. Stroma production is stimulated by cancer-cell derived growth factors including transforming growth factor-β (TΘPβ), hepatocyte growth factor (HGF), fibroblast growth factor (FGF), insulin-like growth factor 1 (IGF-1) and epidermal growth factor (EGF). The desmoplastic reaction is composed of extracellular matrix (ECM) proteins, primarily type I and Ill collagen, fibronectin and proteoglycans; small endothelium lined vessels; and a diverse population of cells including inflammatory cells, fibroblasts and stellate cells. The stroma can form up to 90% of the tumor volume, a property which is unique to pancreatic cancer. The tumor microenvironment in pancreatic cancer plays a role in its chemoresistance.

Chronic inflammation results in fibrosis and chronic fibrosis also predisposes to cancer initiation. In addition to cancer-induced chronic inflammation as a driver of fibrosis, cancer treatments also play an important role in creating the fibrotic tumor microenvironment (TME). Organ fibrosis, most notably pulmonary fibrosis, is a known toxicity of multiple chemotherapeutic agents including bleomycin, gemcitabine, and methotrexate. In vitro and in vivo studies demonstrate chemotherapy may promote an inflammatory and fibrotic microenvironment likely through tissue injury related to oxidative stress. Tissues exposed to chemotherapy undergo similar stages of wound healing including inflammation with influx of immune cells, followed by fibroblast activation and proliferation and remodeling which involves the accumulation and cross-linking of ECM.

Cancer develops within a complex microenvironment critical to supporting tumor survival, growth, and metastasis. This tumor microenvironment (TME) is composed of a web of vasculature, extracellular matrix (ECM), stromal cells, immune cells, and soluble signaling molecules which form a dynamic “organ” critical to the pathophysiology of cancer. Within the TME, cancer-associated fibrosis has emerged as a critical regulator of cancer behavior. Indeed, fibrosis is a hallmark of cancer. Up to 20% of cancers are linked to chronic inflammation-related fibrosis (either from infectious or autoimmune etiologies) including hepatocellular, gastric, esophageal, head and neck, colon, pancreatic, cervix, and vulvar cancers.

Fibrosis has been reported to support cancer growth through a variety of mechanisms including direct cellular interactions, immune modulation, and ECM remodeling. As a stromal progenitor cell, significantly impact the formation of the TME and are important mediators of fibrosis.

Fibrosis is important in not only established tumor sites but also in the creation of a premetastatic niche. In vivo models demonstrate increased fibronectin expression in the stroma of future metastatic sites.

Fibrosis may be monitored, e.g. in diagnosis, during and subsequent to treatment to assess the efficacy of treatment. The presence of fibrosis can be detected by means known in the art, for example by examination of tissue for excess scarring. Prior to fibrosis, an individual may be determined to be susceptible based on undesirable increase in inflammatory mediators that can exacerbate tissue injury, such as IL-1, TNF-α and reactive oxygen and nitrogen species. Profibrotic mediators such as TGF-β1 may be present. Also present are activated myofibroblasts, which may be resistant to induction of apoptosis. Methods of monitoring fibrosis may include, for example, chest X-ray that image the scar tissue typical of pulmonary fibrosis, and it may be useful for monitoring the course of the illness and treatment. Computerized tomography (CT) scan to combine X-ray images taken from many different angles to produce cross-sectional images of internal structures in the body. A high-resolution CT scan can be particularly helpful in determining the extent of lung damage caused by pulmonary fibrosis. An echocardiogram uses sound waves to visualize the heart. Functional tests can be useful, e.g. with pulmonary fibrosis, including pulmonary function tests, e.g. spirometry, pulse oximetry, exercise stress test, arterial blood gas test, etc.

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

In some embodiments the cancer is a solid cancer, involved with fibrosis. In some embodiments the cancer is a lung cancer, e.g. lung carcinoma.

Lung carcinoma is the leading cause of cancer-related death worldwide. About 85% of cases are related to cigarette smoking. Symptoms can include cough, chest discomfort or pain, weight loss, and, less commonly, hemoptysis; however, many patients present with metastatic disease with or without any clinical symptoms. The diagnosis is typically made by chest x-ray or CT and confirmed by biopsy. Depending on the stage of the disease, treatment includes surgery, chemotherapy, radiation therapy, or a combination. For the past several decades, the prognosis for a lung cancer patient was poor, with only 15% of patients surviving >5 years from the time of diagnosis. For patients with stage IV (metastatic) disease, the 5-year overall survival rate was <1%. However, outcomes have improved because of the identification of certain mutations that can be targeted for therapy and current 5-year survival rates are 19% (23% for women and 16% for men).

Chronic inflammation increases the risk of many cancers, including lung cancer. For example, COPD (chronic obstructive pulmonary disease), alpha-1 antitrypsin deficiency, and pulmonary fibrosis increase susceptibility to lung cancer. People whose lungs are scarred by other lung diseases (eg, tuberculosis) are at increased risk of lung cancer. Also, active smokers who take beta-carotene supplements may have an increased risk of developing lung cancer.

In some patients with lung cancer, secondary or additional mutations in genes that stimulate cell growth (K-ras, MYC) cause abnormalities in growth factor receptor signaling (EGFR, HER2/neu) and inhibit apoptosis and can contribute to the uncontrolled proliferation of abnormal cells. In addition, mutations that inhibit tumor-suppressor genes (eg, p53, APC) can lead to cancer. Other mutations that may be responsible include the EML-4-ALK translocation and mutations in ROS-1, BRAF, and PI3KCA. Genes such as these that are primarily responsible for lung cancer are called oncogenic driver mutations. Although oncogenic driver mutations can cause or contribute to lung cancer among smokers, these mutations are particularly likely to be a cause of lung cancer among never-smokers.

Lung cancer is classified into 2 major categories: Small cell lung cancer (SCLC), about 15% of cases; and non-small cell lung cancer (NSCLC), about 85% of cases. SCLC is highly aggressive and almost always occurs in smokers. It is rapidly growing, and roughly 80% of patients have metastatic disease at the time of diagnosis. The clinical behavior of NSCLC is more variable and depends on histologic type, but about 40% of patients will have metastatic disease outside of the chest at the time of diagnosis. Oncogenic driver mutations have been identified primarily in adenocarcinoma, and attempts are being made to identify similar mutations in squamous cell carcinoma (eg, FGFR1, DDR2, and PI3K). NSCLC has 4 stages, I through IV (using the TNM system). TNM staging is based on tumor size, tumor and lymph node location, and the presence or absence of distant metastases.

For NSCLC, the 5-year survival rate varies by stage, from 60 to 70% for patients with stage I disease to <1% for patients with stage IV disease. On average, untreated patients with metastatic NSCLC survive 6 months, whereas the median survival for treated patients is about 9 months. Recently, patient survival has improved in both early and later stage NSCLC. Evidence shows improved survival in early-stage disease (stages IB to IIIB) when platinum-based chemotherapy regimens are used after surgical resection. In addition, targeted therapies have improved survival in patients with stage IV disease, in particular patients with an EGFR mutation, EML-4-ALK and ROS-1 translocations. Targeted therapies and improved sequential treatments are incrementally prolonging survival, particularly in later stage disease.

Treatment of lung cancer varies by cell type and by stage of disease. Many patient factors not related to the tumor affect treatment choice. Poor cardiopulmonary reserve, undernutrition, frailty or poor physical performance status (assessed by, eg, Karnofsky performance status [KPS] or Eastern Cooperative Oncology Group performance status [ECOGPS]), comorbidities, including cytopenias, and psychiatric or cognitive illness all may lead to a decision for palliative over curative treatment or for no treatment at all, even though a cure with aggressive therapy might technically be possible.

Immunotherapy harnesses the body's immune system to eliminate the cancer, and is used to treat advanced stage (IV) non-small cell lung cancer if there is high programmed cell death protein 1 (PD-1) or PDL-1 expression.

Treatment for NSCLC typically involves assessment of eligibility for surgery followed by choice of surgery, chemotherapy, radiation therapy, or a combination of modalities as appropriate, depending on tumor type and stage. For stage I and II disease, the standard approach is surgical resection with either lobectomy or pneumonectomy combined with mediastinal lymph node sampling or complete lymph node dissection. Patients with early-stage disease who are high-risk surgical candidates may instead have local, non-surgical treatment, such as radiation therapy (stereotactic or conventional) or radiofrequency ablation. Adjuvant chemotherapy after surgery is now standard practice for patients with stage II or stage Ill disease and possibly also for patients with stage IB disease and tumors >4 cm. A commonly used chemotherapy regimen is a cisplatin-based doublet (combination of a cisplatin and another chemotherapy drug, such as vinorelbine, docetaxel, paclitaxel). Neoadjuvant (preoperative) chemotherapy in early-stage NSCLC is also commonly used and consists of 4 cycles of a cisplatin-doublet. In patients who cannot receive cisplatin, carboplatin can be substituted. Multiple trials are examining neoadjuvant treatment with immunotherapy drugs. Stage Ill disease is treated with either chemotherapy, radiation therapy, surgery, or a combination of therapies; the sequence and choice of treatment depend on the location of the patient's disease and comorbidities. In stage IV disease, prolonging survival and palliation of symptoms are the goals. Chemotherapy, targeted drugs, and radiation therapy may be used to reduce tumor burden, relieve symptoms, and improve quality of life.

NSCLC treatment can be based on precision medicine. Molecular analysis is done on adenocarcinomas to look for specific mutations that can direct therapy. Several immune oncology drugs (nivolumab, pembrolizumab, durvalumab, and atezolizumab) are available for NSCLC treatment. These drugs stimulate immune responsiveness, assist in the cancer being recognized as foreign, and inhibit the tumor's ability to block the natural immune system response. For example, tumors that have high expression of the PD-L1 protein are responsive to pembrolizumab treatment. For tumors bearing an oncogenic driver mutation, targeted treatments are used first. In stage IV patients with sensitive EGFR mutations (ie, deletion exon 19, exon 21 L858 mutation), EGFR tyrosine kinase inhibitors (TKIs) may be given as first-line therapy; response rates and progression-free survival are better than those obtained using standard chemotherapy. EGFR TKIs include gefitinib, erlotinib, afatinib, and brigatinib. Osimertinib is the treatment of choice for EGFR mutant NSCLC that has an acquired T790M mutation. In patients with nonsquamous NSCLC without an oncogenic driver mutation, bevacizumab, a vascular endothelial growth factor inhibitor, can be used in combination with standard chemotherapy (eg, a platinum-based doublet, such as carboplatin plus paclitaxel) to improve outcomes. Necitumumab is now available for use in combination with cisplatin plus gemcitabine for first-line treatment of NSCLC squamous cell carcinoma. Patients who have EML-4-ALK translocations should receive an ALK and ROS-1 inhibitor (crizotinib, ceritinib, or alectinib). Patients with ALK mutations can be given alectinib or ceritinib. Patients with BRAF mutations benefit from the BRAF inhibitors (eg, dabrafenib, trametinib). Many other targeted biologic agents are under investigation, including some that specifically target cancer cell signal transduction pathways or the angiogenesis pathways that supply oxygen and nutrition to growing tumor cells.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting PD-L1 and those targeting CTLA-4 such as ipilimumab.

Antibiotics, e.g. antibiotics with the classes of aminoglycosides; carbapenems; and the like; penicillins, e.g. penicillin G, penicillin V, methicillin, oxacillin, carbenicillin, nafcillin, ampicillin, etc. penicillins in combination with Q-lactamase inhibitors, cephalosporins, e.g. cefaclor, cefazolin, cefuroxime, moxalactam, etc; tetracyclines; cephalosporins; quinolones; lincomycins; macrolides; sulfonamides; glycopeptides including the anti-infective antibiotics vancomycin, teicoplanin, telavancin, ramoplanin and decaplanin. Derivatives of vancomycin include, for example, oritavancin and dalbavancin (both lipoglycopeptides). Telavancin is a semi-synthetic lipoglycopeptide derivative of vancomycin (approved by FDA in 2009). Other vancomycin analogs are disclosed, for example, in WO 2015022335 A1 and Chen et al. (2003) PNAS 100(10): 5658-5663, each herein specifically incorporated by reference. Non-limiting examples of antibiotics include vancomycin, linezolid, azithromycin, daptomycin, colistin, eperezolid, fusidic acid, rifampicin, tetracyclin, fidaxomicin, clindamycin, lincomycin, rifalazil, and clarithromycin.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

The definition of an appropriate patient sample encompasses blood and other liquid samples of biological origin, solid tissue samples such as a biopsy specimen or tissue cultures or cells derived there from and the progeny thereof. The definition also includes samples that have been manipulated in any way after their procurement, such as by treatment with reagents; washed; or enrichment for certain cell populations, such as endometrial cells, kidney disease cells, inflammatory disease cells and/or transplant rejection (GVHD) cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample comprising polynucleotides and/or polypeptides that is obtained from a patient's sample cell (e.g., a cell lysate or other cell extract comprising polynucleotides and/or polypeptides); and a sample comprising sample cells from a patient. A biological sample comprising a sample cell from a patient can also include normal, non-diseased cells.

The term “diagnosis” is used herein to refer to the identification of a molecular or pathological state, disease or condition, such as the identification of fibrosis. The methods of the invention may further comprise analysis of fibrosis or fibrotic activity following treatment according to the methods as claimed. Analysis of fibrosis may be made by obtaining a biological sample and examining molecular or pathological state, disease or condition, and the like.

As used herein, the terms “treatment,” “treating,” and the like, refer to administering an agent, or carrying out a procedure for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, covers any treatment of fibrosis in a mammal, particularly in a human, and includes: (a) preventing the development of fibrosis; (b) inhibiting ongoing fibrosis, i.e., arresting its development; and (c) relieving fibrosis, i.e., causing regression of fibrosis.

Treating may refer to any indicia of success in the treatment or amelioration or prevention of fibrosis or cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with fibrosis. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic (i.e., first therapeutic agent) and the compounds as used herein. When administered in combination, each component can be administered at the same time or sequentially in any order at different points in time. Thus, each component can be administered separately but sufficiently closely in time so as to provide the desired therapeutic effect. First therapeutic agents contemplated for use with the methods of the present invention include any other agent for use in the treatment of fibrosis. Examples of such therapeutic agents include but are not limited anti-fibrotic agents.

“Concomitant administration” of a known therapeutic agent with a pharmaceutical composition of the present invention means administration of the therapeutic agent and inhibitor agent at such time that both the known therapeutic agent and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention. Therapeutic agents contemplated for concomitant administration according to the methods of the present invention include any other agent for use in the treatment of fibrosis.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

A CRISPR/Cas protein (also referred to herein as a CRISPR/Cas endonuclease) interacts with (binds to) a corresponding guide RNA to form a ribonucleoprotein (RNP) complex (referred to herein as a CRISPR/Cas complex) that is targeted to a particular site (a target sequence) in a target genome via base pairing between the guide RNA and a target sequence within the target genome. A guide RNA includes (i) a nucleotide sequence (a guide sequence) that is complementary to a sequence (the target site) of a target DNA and (ii) a protein-binding region that includes a double stranded RNA (dsRNA) duplex and bind to a corresponding CRISPR/Cas protein. The guide RNA can be readily modified in order to target any desired sequence within a target genome (by modifying the guide sequence). A wild type CRISPR/Cas protein (e.g., a Cas9 protein) normally has nuclease activity that cleaves a target nucleic acid (e.g., a double stranded DNA (dsDNA)) at a target site defined by the region of complementarity between the guide sequence of the guide RNA and the target nucleic acid. The term “CRISPR/Cas protein,” as used herein, includes wild type CRISPR/Cas proteins, and also variant CRISPR/Cas proteins, e.g., CRISPR/Cas proteins with one or more mutations in a catalytic domain rendering the protein a nickase.

A target DNA (e.g., genomic DNA) that can be recognized and cleaved by a CRISP/Cas protein (e.g., Cas9) is a DNA polynucleotide that comprises a “target site” or “target sequence.” The terms “CRISPR/Cas target site” or “CRISPR/Cas target sequence” are used interchangeably herein to refer to a nucleic acid sequence present in a target DNA (e.g., genomic DNA of a cell) to which a CRISPR/Cas guide RNA can bind, allowing cleavage of the target DNA by the CRISPR/Cas endonuclease. The strand of the target DNA that is complementary to and hybridizes with the CRISPR/Cas guide RNA is referred to as the “complementary strand” or the “target strand” and the strand of the target DNA that is complementary to the “complementary strand” (and is therefore not complementary to the guide RNA) is referred to as the “non-complementary strand” or “non-target strand.” A target sequence can be any desired length and, in some cases, can depend upon the type of CRISPR/Cas guide RNA and CRISPR/Cas protein that will be used to target the target sequence.

A wild type CRISPR/Cas protein (e.g., Cas9 protein) normally has nuclease activity that cleaves a target nucleic acid (e.g., a double stranded DNA (dsDNA)) at a target site defined by the region of complementarity between the guide sequence of the guide RNA and the target nucleic acid. In some cases, site-specific targeting to the target nucleic acid occurs at locations determined by both (i) base-pairing complementarity between the guide nucleic acid and the target nucleic acid; and (ii) a short motif referred to as the “protospacer adjacent motif” (PAM) in the target nucleic acid. For example, when a Cas9 protein binds to (in some cases cleaves) a dsDNA target nucleic acid, the PAM sequence that is recognized (bound) by the Cas9 polypeptide is present on the non-complementary strand (the strand that does not hybridize with the targeting segment of the guide nucleic acid) of the target DNA. CRISRPR/Cas (e.g., Cas9) proteins from different species can have different PAM sequence requirements.

A nucleic acid that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

As used herein, “CRISPER/Cas9 system” typically includes a polynucleotide sequence (which may, together, be referred to as an expression cassette, or one or more sgRNAs may be separately encoded and referred to as a cassette), wherein the polynucleotide sequence encodes the Cas9 nuclease alone, or also encodes one or more single guide RNAs (sgRNAs) as well as the Cas9 nuclease. In some instances, the expression cassette encoding the CRISPER/Cas9 system is referred to as a “transgene.”

### Methods

Individuals diagnosed or at risk of developing a fibrotic disease or cancer, e.g. cancer associated with fibrosis, are treated by administering an agent that inhibits CD63 pathways. In some embodiments the fibrosis is a pulmonary fibrosis, such as idiopathic pulmonary fibrosis In some embodiments, the fibrosis is skin fibrosis, kidney fibrosis, liver fibrosis such as liver cirrhosis, non-alcoholic steatohepatitis (NASH), and non-alcoholic fatty liver disease (NHFLD). When the fibrosis is kidney fibrosis, the kidney fibrosis may be caused by hypertension, diabetes, obstruction, or infection. In some embodiments the fibrosis is associated with cancer and tumor growth, i.e. tumor related tissue fibrosis, including without limitation lung cancer, skin cancer, sarcoma, etc. In other embodiments the fibrosis is associated with chronic inflammation or injury such as irradiation in tissues, including without limitation fibrosis of liver, lung, kidney, uterus, the eye and the lens, IgG4-related disease, chronic GvHD, and the like.

Methods include administering to a subject in need of treatment a therapeutically effective amount or an effective dose of a therapeutic entity (e.g., inhibitor agent) of CD63. In some embodiments, effective doses of the therapeutic entity of the present invention described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

In some embodiments reduction in fibrosis is monitored during or subsequent to treatment. Monitoring may include, without limitation, detecting a decrease in fibrotic cells, decrease in fatty infiltrating cells, etc.

An anti-CD63 agent for use in the methods of the invention blocks or downregulates CD63. For example the agent may block interactions with CD63 counter-receptors (ligands). In some embodiments, an anti-CD63 agent interferes with binding between CD63 present on the fibrotic or cancer cell and co-receptors present on a phagocytic cell, e.g. integrins, etc. Such methods increase phagocytosis of the fibrotic cell. Suitable anti-CD63 agents anti-CD63 antibodies, small molecules, soluble receptors, and the like, where the term antibodies encompasses antibody fragments, ISVs, and variants thereof, as known in the art. In some embodiments the anti-CD63 agent is an anti-CD63 antibody. In some embodiments, the anti-CD63 antibody is anti-CD63 clone H5C6 produced by Novus.

The effective dose of an anti-CD63 agent can vary with the agent, but will generally range from up to about 50 mg/kg, up to about 40 mg/kg, up to about 30 mg/kg, up to about 20 mg/kg, up to about 10 mg/kg, up to about 5 mg/kg; up to about 1 mg/kg, up to about 0.5 mg/kg; up to about 0.1 mg/kg; up to about 0.05 mg/kg; where the dose may vary with the specific antibody and recipient.

The anti-CD63 agent may be administered one or a plurality of days, and in some embodiments is administered daily, every two days, semi-weekly, weekly, etc. for a period of from about 1, about 2, about 3, about 4, about 5, about 6, about 7 or more weeks, up to a chronic maintenance level of dosing.

In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still yet some other embodiments, for prophylactic applications, pharmaceutical compositions or medicaments are administered to a patient susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

In still yet some other embodiments, for therapeutic applications, therapeutic entities of the present invention are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved.

Examples of symptoms, illnesses, and/or diseases that can be treated with an anti-CD63 agent include, but are not limited to cancer. As used herein “cancer” includes any form of cancer, including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; and brain cancers, including minimal residual disease, and including both primary and metastatic tumors. Any cancer is a suitable cancer to be treated by the subject methods and compositions.

Lung cancer, e.g. small cell lung cancer and non-small cell lung cancer (NSCLC), are of particular interest. NSCLC includes, for example, adenocarcinoma, squamous cell carcinoma, small cell lung carcinoma and large cell carcinoma.

Carcinomas are malignancies that originate in the epithelial tissues. Epithelial cells cover the external surface of the body, line the internal cavities, and form the lining of glandular tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc.

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

According to the present invention, compositions can be administered by parenteral, topical, intravenous, oral, subcutaneous, intraarterial, intracranial, intraperitoneal, intranasal or intramuscular means. The most typical route of administration is intravenous although other routes can be equally effective.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. An exemplary composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998. The pharmaceutical compositions are generally formulated as sterile, substantially isotonic and in full compliance with all Good Manufacturing Practice (GMP) regulations of the U.S. Food and Drug Administration. Preferably, a therapeutically effective dose will provide therapeutic benefit without causing substantial toxicity.

Toxicity of the proteins described herein can be determined by standard pharmaceutical procedures in cell cultures or experimental animals, e.g., by determining the LD50 (the dose lethal to 50% of the population) or the LDi00 (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

Also within the scope of the invention are kits comprising the compositions of the invention and instructions for use. The kit can further contain a least one additional reagent. Kits typically include a label indicating the intended use of the contents of the kit. The term label includes any writing, or recorded material supplied on or with the kit, or which otherwise accompanies the kit.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims. Those skilled in the art will recognize, or be able to ascertain using no more than routine experimentation, many equivalents to the specific embodiments of the invention described herein. Such equivalents are intended to be encompassed by the appended claims.

### Gene Therapy Formulations

In some embodiments, an anti-CD63 agent for use in the methods of the invention is a therapeutic gene therapy vector. In some embodiments, the therapeutic gene therapy vector is an AAV virus comprising a therapeutic sequence to downregulate, e.g. delete, CD63. In some embodiments, the therapeutic vector comprises a CRISPR/Cas9 system and at least one guide RNA (gRNA) directed to a CD63 gene for deletion. In some embodiments, the therapeutic vector comprises short hairpin RNA (shRNA) targeting CD63 gene for deletion.

AAV gene therapy. In some embodiments, the vector is a recombinant adeno-associated virus (AAV) vector. AAV vectors are DNA viruses of relatively small size that can integrate, in a stable and site specific manner, into the genome of the cells that they infect. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134: 52-63), while AAV1-4 and AAV6 are all found in the study of adenovirus (Ursula Bantel-Schaal, Hajo Delius and Harald zur Hausen. J. Viral., 1999. 73: 939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. ME Bloom, RMLinden, C RParrish, Eds.) p 5-14, Rudder Arnold, London, U K (2006); and D E Bowles, J E Rabinowitz, R J Samulski “The Genus Dependovirus” (J R Kerr, SF Cotmore. ME Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

A cell type-specific promoter allows precise manipulation of gene expression without affecting other cell types. Cell types of particular interest are fibroblasts, cancer associated fibroblasts (CAFs), lung carcinoma cells, osteosarcoma cells, and hepatocellular carcinoma cells. Aspects of the present invention encompass expression cassettes and/or vectors comprising polynucleotide sequences of interest for expression in targeted cells. The polynucleotides can comprise promoters operably linked to sgRNAs directed to CD63 coding sequence. Targeted expression is accomplished using a cell-selective or cell-specific promoter. For example, promoters specific to fibroblasts, cancer associated fibroblasts (CAFs), lung carcinoma cells, osteosarcoma cells, and hepatocellular carcinoma cells may be used. When targeted expression is desired in fibroblasts then any fibroblast specific promoter may be used. These promoters include, without limitation, type I collagen alpha chain 1 (COL1A1), type I collagen alpha chain 2 (COL1A2), human integrin all subunit, fibroblast specific protein 1 (FSP1), etc. When targeted expression is desired in hepatocellular carcinoma cells then any hepatocellular carcinoma cell specific promoter may be used. For example, promoters described in Foka et al. J Gene Med. 2010 December; 12(12):956-67, herein specifically incorporated by reference. When targeted expression is desired in lung carcinoma cells then any lung carcinoma cell specific promoter may be used. For example, promoters described in Fukazawa et al. Mol Cancer Ther. 2007 January; 6(1):244-52, herein specifically incorporated by reference. When targeted expression is desired in osteosarcoma cells then any osteosarcoma cell specific promoter may be used. For example, promoters described in Pollmann et al. Int J Mol Med. 2004 October; 14(4):737-42, herein specifically incorporated by reference.

Methods for treating a mammalian patient for fibrosis are provided. Methods for treating a mammalian patient for cancer are also provided. Aspects of the methods include a administering an anti-CD63 agent. Anti-CD63 agents may be in the form of a therapeutic gene therapy which include a mammalian viral vector, comprising a promoter specific to the cancer or fibrosis that it is directed to, that promotes expression of a transgene specifically in cell types of interest, such as fibroblasts, cancer associated fibroblasts (CAFs), lung carcinoma cells, osteosarcoma cells, or hepatocellular carcinoma cells, said promoter in operable linkage with an expression cassette encoding the transgene, wherein the expressed transgene inhibits activity of an expression product of an endogenous CD63 gene. Aspects of the methods include local administration to the tumor or systemic administration to treat the subject for the cancer. A variety of cancers may be treated by practicing the methods, including lung cancer, liver cancer, or sarcoma. Aspects of the methods include local administration to the fibrotic tissue or systemic administration to treat the subject for the fibrosis. A variety of fibroses may be treated by practicing the methods, including skin fibrosis, liver cirrhosis, non-alcoholic steatohepatitis (NASH), non-alcoholic fatty liver disease (NHFLD), or kidney fibrosis.

In some embodiments of the composition, the transgene encodes a CRISPER/Cas9 system. In some embodiments, the transgene encodes a short hairpin RNA (shRNA) targeting CD63 gene.

### EXPERIMENTAL

### Example 1

Fibroblasts protect tumor cells from phagocytosis through CD63 in lung cancer.

Lung cancer is the leading cause for cancer-related deaths worldwide. Despite recent advances and improved therapeutic options, overall survival remains poor. Tetraspanins are found in extracellular vesicles and contribute to protein trafficking. Here, we investigated whether fibroblasts drive lung cancer through tetraspanins. After demonstrating increased CD63 expression both in lung cancer and idiopathic pulmonary fibrosis, we show that fibroblasts use extracellular vesicles to transport CD63 to the plasma membrane of cancer cells. Next, we show in vitro that inhibiting CD63 increases phagocytosis of lung cancer cells and lung fibroblasts and that CD63 co-localizes with CD9 on macrophages. Finally, inhibiting CD63 eliminates tumor cells in an adaptive transfer model. Next to its insights how fibroblast affect cancer cells, our study suggests that CD63 might be a target for immunotherapy in lung cancer.

Cancer cells can protect themselves from macrophage-mediated phagocytosis through “don't-eat-me-signals” such as CD47. Immune cells form together with fibroblasts the tumor stroma. Fibroblasts contribute to tumor progression and therapy resistance. However, the underlying mechanisms are only incompletely understood. Our group's focuses are fibrotic diseases, especially IPF, and the innate immune system. Recently, we have demonstrated that the transcription factor JUN drives IPF through adjusting adaptive immunity and that CD47 is upregulated in scleroderma to protect diseased fibroblasts from phagocytosis.

In this study, we investigated whether fibroblasts drive tumor progression through tetraspanins. To answer this hypothesis, we split our investigation into three parts respectively questions. First, whether lung cancer and IPF increase a specific tetraspanin. Second, whether this tetraspanin functionally links fibroblasts with cancer cells. And third, whether this tetraspanin affects the innate immune system.

To answer the first question, whether both IPF and lung cancer increase a specific tetraspanin, we analyzed lung cancer and normal lung for the expression of CD9, CD63, CD81, and CD151 through immunohistochemistry and mass spectroscopy. While CD9 was the most abundantly expressed tetraspanin both in normal and cancer tissue and CD81 was more widely expressed in normal tissue, CD63 was the only tetraspanin with a stronger expression in lung cancer. As IPF patients more often develop lung cancer, we hypothesized that lung cancer and IPF tissue share patterns in the expression of tetraspanins that support tumor progression. Hence, we run an exploratory mass spectroscopy analysis on sorted lung tissue from three IPF patients and confirmed its finding with IHC. In accordance with our previous observations, CD63 was more strongly expressed in these IPF samples. As its expression was increased both in lung cancer and IPF, we chose CD63 for a further exploration.

The next question addressed how CD63 functionally links fibroblasts with lung cancer cells. To analyze this relationship in vitro, we established primary lung fibroblast cultures from the tumor of a lung cancer patient (luc-fb) and a IPF patient (ipf-fb), generated a primary lung cancer culture (LC19), and used an established cell line, A549, as a second lung cancer cell line. We used healthy lung tissue from a lung cancer patient to generate normal pulmonary fibroblast cultures (npu-fb). We first used IF to analyze general CD63 expression in these fibroblasts. Both lung cancer and IPF fibroblasts expressed significantly more CD63 than normal fibroblasts. While most CD63 was expressed inside the cells, we assumed that a tumor supportive function of CD63 in fibroblasts is mediated through its expression on the cell surface. Therefore, we analyzed most subsequent experiments by flow cytometry. After observing that pro-inflammatory cytokines tendentially decrease CD63, we used co-cultures to investigate whether the adaptive and the innate immune system affect CD63 surface expression of lung fibroblasts and lung cancer cells. While neutrophils did not affect CD63, T cells decreased CD63. We then hypothesized that fibroblasts increase CD63 through the secretion of CD63-containing EV and the subsequent fusion of these EV with the cell membrane of cancer cells. In accordance with this hypothesis, co-culture of A549 with lung cancer fibroblasts increased CD63 in A549 cells and an ELISA showed secretion of CD63 into the supernatant through lung cancer fibroblasts. In addition, we transfected luc-fb with a plasmid containing GFP-labeled CD63. Supporting our hypothesis, culturing A549 directly with eGFP-CD63+ fibroblasts or only with the supernatant from eGFP-CD63+ fibroblasts led to GFP positivity of A549 cells in flow cytometry.

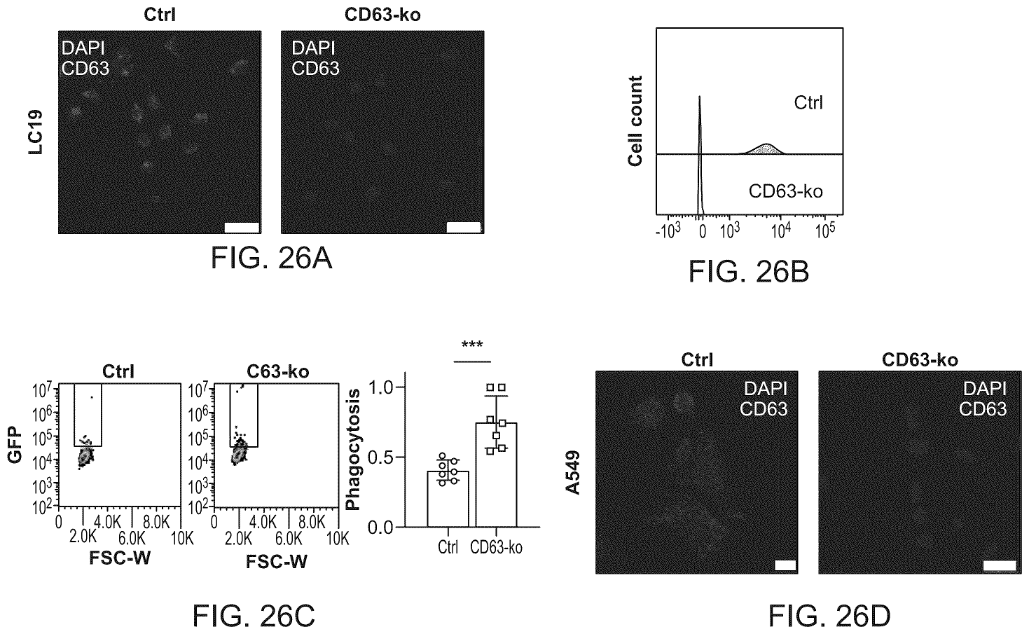

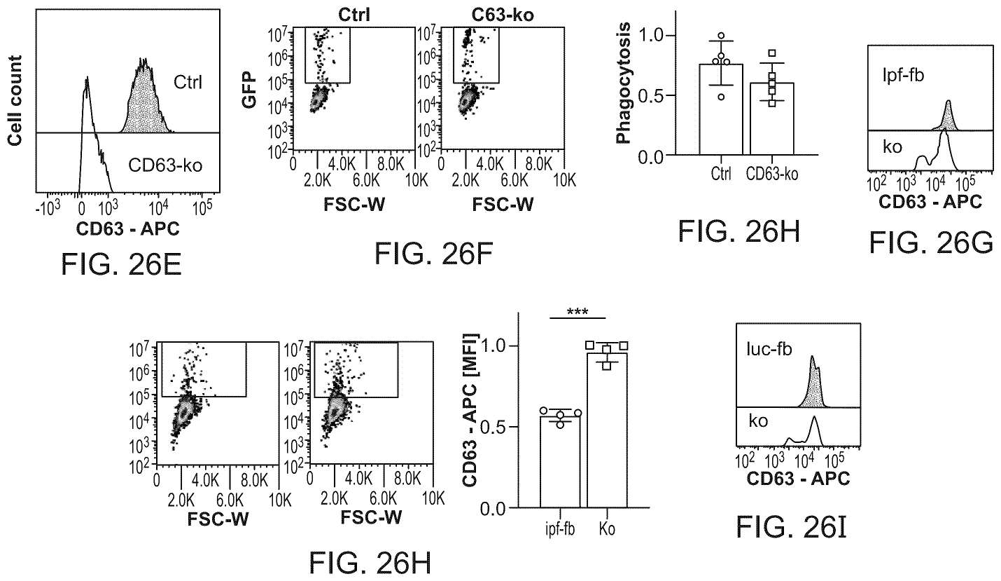

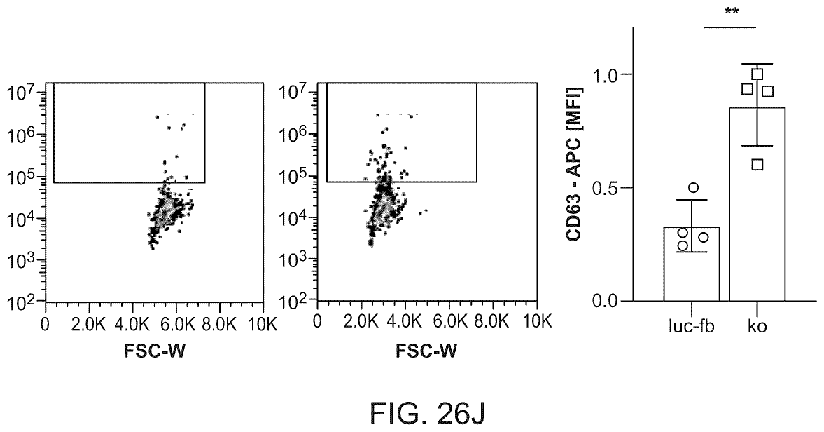

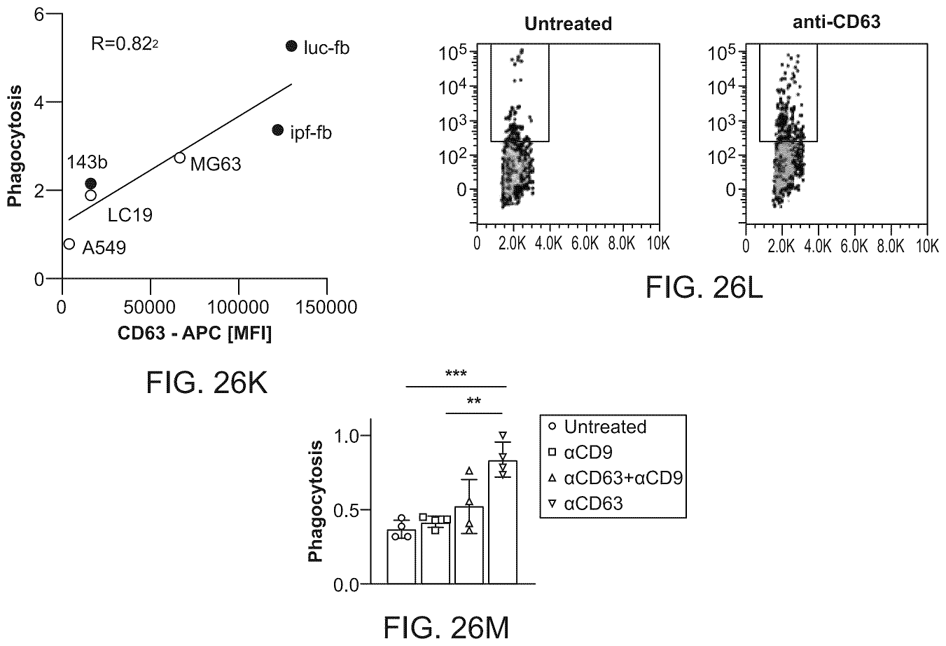

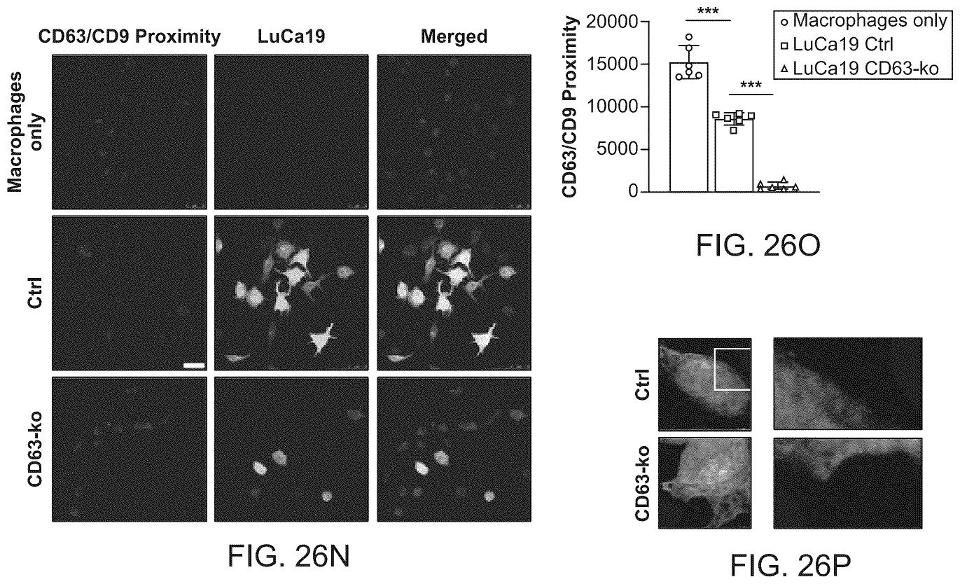

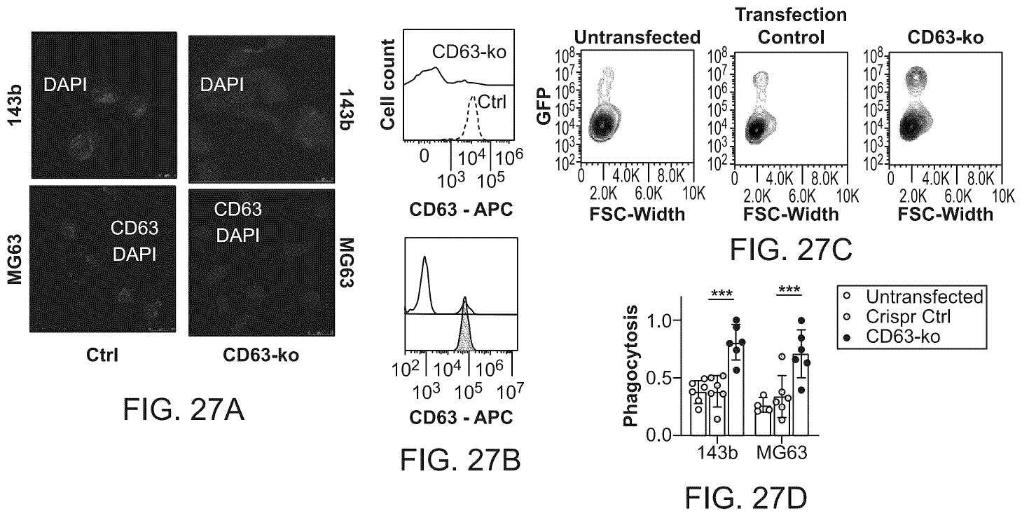

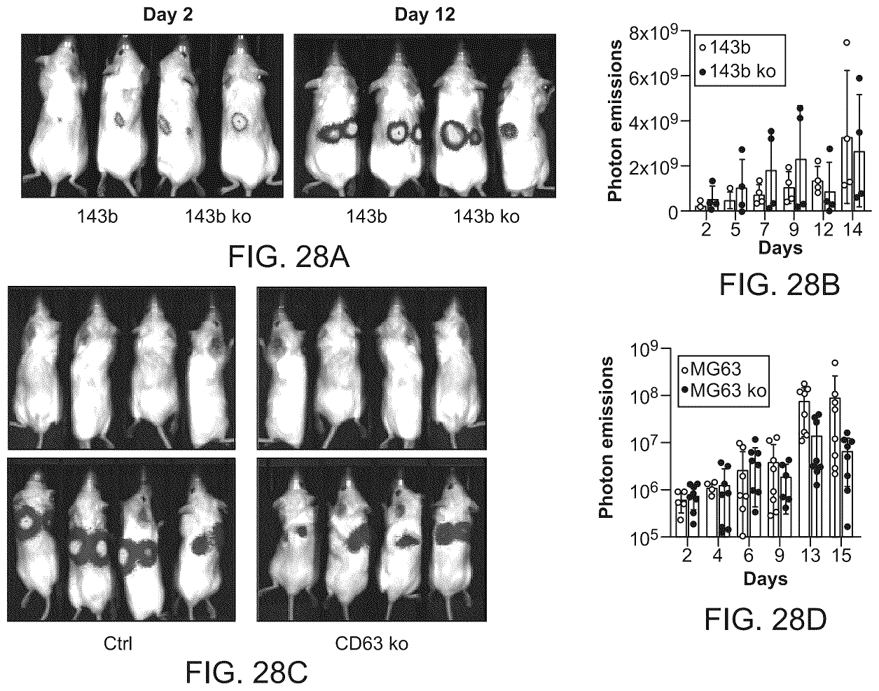

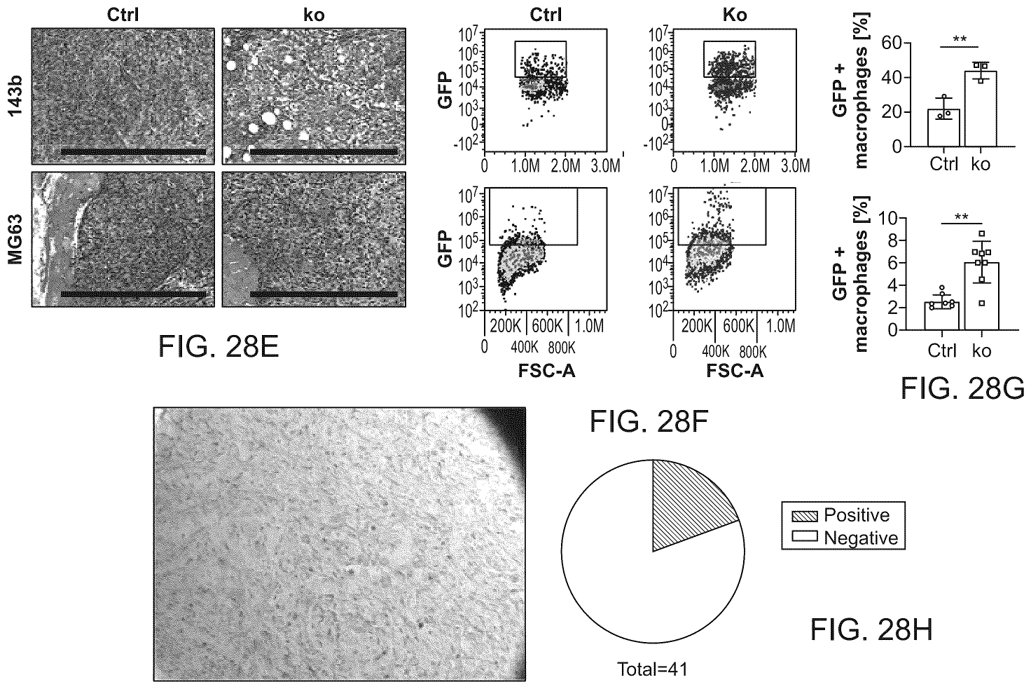

After having determined how surface CD63 is regulated and how CD63 on fibroblasts can affect cancer cells, we investigated if CD63 conveys a specific tumor supportive function. For this purpose, we established CD63 knockout cultures of LC19 and A549 cells through Cas9/sgRNA transfection and subsequent cell sorting (FIG. 26 A, B, D, E). After observing that a CD63 knockout decreased proliferation of both LC19 and A549, we run in vitro phagocytosis assays. While phagocytosis increased in LC19 after CD63 knockout, the CD63 knockout did not affect the phagocytosis of A549 (FIG. 26 C, F). Hypothesizing that CD63 generally regulates phagocytosis, we also performed a CD63 knockout in luc- and IPF fibroblasts. Though the knockout of CD63 was only partial after MACS sorting, phagocytosis increased after CD63 knockout in both fibroblast types (FIG. 26 G-J). To determine whether this increased phagocytosis was dependent on the cellular origin, we run in vitro phagocytosis assays with mesenchymal osteosarcoma cells, MG63 and 143, which also demonstrated an increase after a CD63 knockout (FIG. 27). When plotting all lung cancer cells, lung fibroblasts, and osteosarcoma cells and the change in phagocytosis after CD63 knockout against the wild-type CD63 expression, higher CD63 expression indicated a more powerful effect of CD63 knockout on phagocytosis (FIG. 26 K). Assuming CD63 works as a “don't eat me signal”, we wondered if CD9 on macrophages works as a binding partner. Blocking CD63 and/or CD9 in luc-fb through antibodies, CD63 blockade increased phagocytosis while combined CD63/CD9 blockade diminished this effect (FIG. 26 L, M). In accordance with the hypothesis that CD63 and CD9 form an axis that regulates phagocytosis, a proximity ligation assay confirmed that CD63 is physically close to CD9 (FIG. 26 N-P).

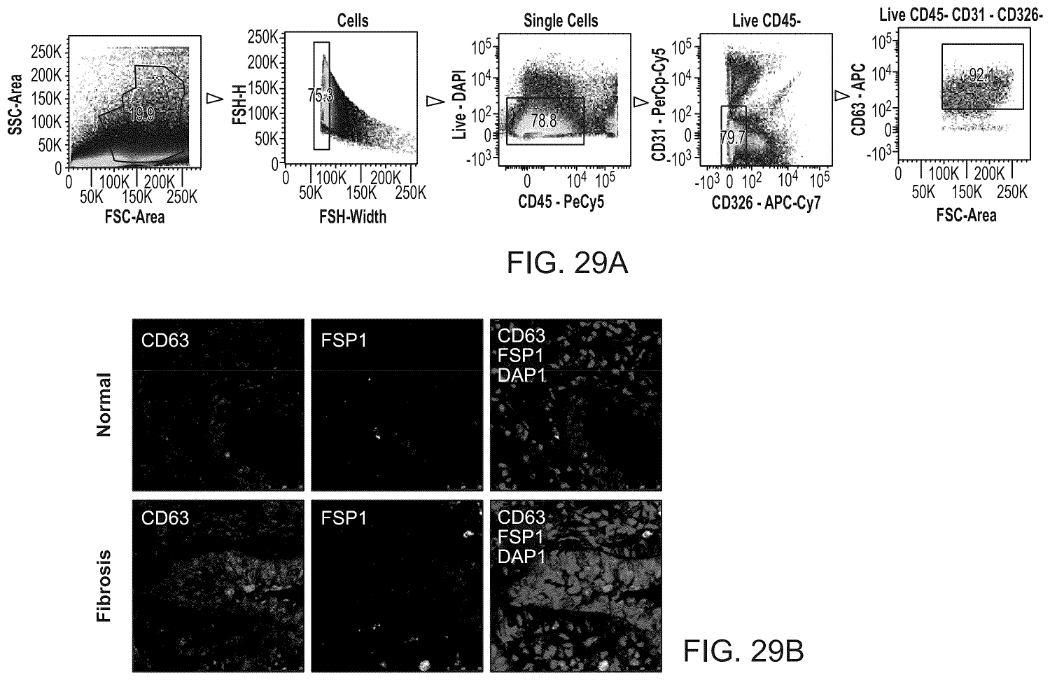

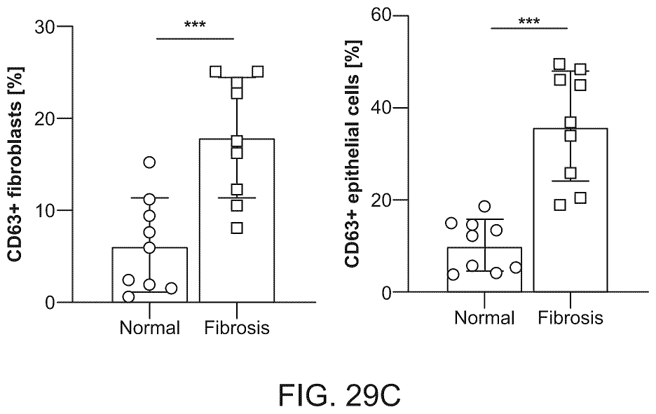

After our in vitro assay, we moved to an adaptive transfer model in which we transplanted cells under the kidney capsule of immunocompromised NSG mice. CD63 knockout led to a decrease in tumor growth in LC19 and A549 cells, a finding that we confirmed with the subcutaneous injection of LC19 cells. As the cell number after a CD63 knockout was too low to measure phagocytosis in vivo, we injected LC19 and A549 subcutaneously and measured phagocytosis through flow cytometry nine days after injection. As in the in vitro experiment, CD63 knockout in LC19 cells increased phagocytosis without having an effect in A549 cells. To determine the dependency on a specific cellular origin, we also injected osteosarcoma cells subcutaneously and measured phagocytosis after 21 days which confirmed our previous results (FIG. 29). Finally, we used a syngeneic lung fibrosis model to determine if lung fibrosis leads to an increase in CD63. Here, lung fibrosis significantly increased CD63 expression both in CD45-Epcam+ and CD45-Epcam-CD31− cells (FIG. 29).

Overall, our study provides new insights into lung cancer biology with therapeutic implications. The tetraspanin CD63 serves as an exosomal marker and participates in intra- and intercellular protein transportation. However, research on other functions of CD63 has been sparse and the role of CD63 as a don't-eat-me-signal has not been explored before. After demonstrating through IHC and mass spectroscopy that CD63 is more expressed in lung cancer than in normal lung, we demonstrate in vitro that CD63 from fibroblasts is transported to the cell membrane of cancer cells through CD63-containing extracellular vesicles. Then, in vitro experiments demonstrate that a CD63 knockout decreases proliferation of lung cancer cells in vitro and increases phagocytosis of LC19 cells and lung fibroblasts. After showing that CD63 physically interacts with CD9 on macrophages, we confirm our in vitro results through ectopic in vivo models in which we demonstrate increased phagocytosis and decreased tumor growth of lung cancer cells after a CD63 knockout.

The observation that blocking CD63 through an antibody also increases phagocytosis supports the assumption that the effect of CD63 as a don't-eat-me-signal is conveyed through its expression on the cell surface. Our study indicates that CD63 is a target for immunotherapy.

### Methods

Husbandry. JUN mice were kept on a regular diet in the facilities of the Veterinary Service Center at Stanford University and had a B6/129 background. Nod.Scid.Gamma mice were purchased from the Jackson Laboratory. Mice were kept on a standard diet. Female and male mice were used. When different sexes were used for individual experiments, groups were sex-matched. Mice were not backcrossed and between 6 and 12 weeks of age during experiments.

Genotyping. To determine the genotype, we harvested tissue from the tail of newborn mice on day 10. We digested the DNA with Quickextract (Lucigen Corporation) at 68° C. for 90 minutes, followed by heat inactivation at 98° C. for five minutes. We ran the genotyping PCR for the Rosa26 and the collagen status with the Phusion® High Fidelity DNA Polymerase (New England Biolabs) and the same primers as described previously.

Adaptive transfer under the kidney capsule. After anesthetizing mice, we areas over both flanks were shaved. After creating a flank cut the subcutaneous tissue was bluntly removed from the underlying soft tissue. The abdominal wall was incised and the kidney luxated out of the abdominal cavity. After piercing the kidney and detaching the renal capsule from the renal tissue, 1,000 cells suspended in Matrigel (MilliporeSigma) were injected under the kidney capsule. Afterwards, the kidney was replaced into the abdominal cavity and the abdominal wall and the skin were separately sutured.

Subcutaneous tumor cell injection. 400,000 cells were resuspended in 100 μl of DMEM and 100 μl of Matrigel (MilliporeSigma). Mice were anesthetized, the back was disinfected, and the cell suspension was slowly subcutaneously injected on both sides of the back. Afterwards, mice were put back into their original cage.

Luciferase-based optimal imaging. We intraperitoneally injected 100 μl of luciferin substrate (15 mg/ml) (Biosynth). 15 minutes later, we performed optical imaging with the Lago optical imaging system (Spectral imaging instruments) and analyzed the images with the Aura Software from the same manufacturer.

Cell culture maintenance. All cell cultures were kept in DMEM (Caisson Labs) supplemented with 5% HPL and 1% Penicillin-Streptomycin (MilliporeSigma) in an incubator at 37° C. 95% O2/5% CO2. Cells were regularly checked for signs for infection and split once being more than 80% confluent. For splitting, cell cultures were washed with PBS and incubated with Trypsin (Gibco) for 5 minutes. The reaction was then quenched with DMEM+ HPL. Cell suspensions were spun down and reapplied into cell dishes. For assays in which we determined CD63 either in the supernatant or on cell surfaces, we used serum-free DMEM/F12 (Gibco) medium supplement with 0.1% PVA (Fisher Scientific), 1% Penicillin-Streptomycin (MilliporeSigma), 10 mM HEPES (Fisher Scientific), 1 μM insulin (MilliporeSigma), and 1 μM transferrin (MilliporeSigma).

Preparation of human platelet lysate (HPL) and HPL-containing medium. Expired human platelets were obtained from the Stanford Blood Bank. Then, platelets were lysed through five quick freeze-thaw cycles. Platelet lysates were then spun down at 4,000 g for 10 minutes, aliquoted into 15 ml tubes and stored at −80° C. For preparing cell mediums, platelet lysates were warmed up, then spun down at 4,000 g for 10 minutes, and sequentially filtered through 0.80, 0.45 and 0.22 μm filters. The final medium, containing DMEM, 5% HPL, 1% Penicillin/Streptomycin and 2 Units of Heparin/ml, was then filtered through a 0.22 μm filter and stored at 4° C.

Harvesting of mouse lungs for subsequent cell cultures or flow cytometry. Mice were euthanized with CO2. The abdomen and chest were disinfected with 70% EtOH, followed by incising the skin from the lower abdomen up to the neck. The thorax was opened, and the heart was flushed with 5 ml of 10 mM EDTA. If tissue was to be used in histology, the trachea was sutured and flushed below with 200 μl of 1% SeaPrep Agarose (Lonza). The lungs were excised, minced with scissors, and filtered through a 70 μm cell strainer (Falcon). Cells were then either used for flow cytometry or cell culture.

Isolation and maintenance of primary mouse lung fibroblast cultures. After filtering cells and tissue as described previously, cells were washed two times with PBS. The supernatant was removed, and cell pellets and tissue parts were transferred into a culture dish. Cells were kept in DMEM+5% HPL supplemented with Ciprofloxacin over five days. Medium was changed on the first and third day. When being 50% confluent, cells were split.

Isolation of primary pulmonary fibroblast and lung cancer cell cultures. Human fibroblasts were obtained discarded fresh lungs tissues from de-identified patients. The tissue was minced with scissors, washed two times with PBS and then plated into a 10 cm cell culture dish in regular cell culture medium (see cell culture maintenance), supplemented with Ciprofloxacin (10 μg/ml) (Bioworld).

Generating human macrophage cultures. Blood was donated by one of the authors. After disinfecting the elbow, blood was drawn into EDTA S-Monovettes® (Sarstedt) with a 21G Vacutainer (BD) connected through a Multi Adapter (Sarstedt). Thereafter, 8 ml of blood was mixed with 32 ml of PBS. Then, the mixture was carefully applied onto 10 ml of Ficoll-Paque Plus (GE) and spun for 30 minutes at room temperature with 400 g with low acceleration and no break. Afterwards, the buffy coat was transferred into a 50 ml tube with transfer pipettes and washed with PBS. After a centrifugation step, cells were plated into 10 cm Petri dish (Corning) in 6 ml of regular DMEM/HPL Medium. After two hours, the medium was replaced by 8 ml DMEM/HPL medium supplemented with 20 ng/ml M-CSF (Gibco). The medium was exchanged on days 3, 5, and 7. The macrophages were used for phagocytosis assays 7 to 9 days after their harvest.

Isolation of human T cells and regulatory T cells. Blood draws and density centrifugations were performed as described above. After washing the buffy coat with PBS, it was washed once again with FACS buffer. Isolation of T cells and regulatory T cells were done with MACS Beads according to the manufacturer's specifications (Miltenyi Biotec). For isolation of CD3+ T cells, cells were labeled with CD3+ beads, and magnetically separated with MS columns (Miltenyi Biotec) on a QuadroMacs Separator (Miltenyi Biotec). For isolating regulatory T cells, first CD4+ T cells were separated through a negative selection, followed by a positive selection of CD25+ T cells on MS columns. Isolated cells were washed with FACS buffer, counted, and used in subsequent experiments.

Isolation of human neutrophils. Blood was drawn as described above. For the isolation of human neutrophils, we used the MACSxpress Whole Blood Neutrophil Isolation Kit (Miltenyi Biotec). After drawing the blood, the whole blood was incubated with the antibody mix for 5 minutes at room temperature in a 15 ml tube on a rotator. Then, the 15 ml tube was placed into the MACSxpress Separator (Miltenyi Biotec) for 15 minutes. The supernatant was collected and washed with PBS. Red blood cells were lysed with ACK lysis buffer (Gibco) for 10 minutes. After washing the cells with PBS, cells were counted and used in subsequent experiments.

Phagocytosis assay in vitro. For measuring phagocytosis through flow cytometry, 10,000 to 25,000 macrophages and fibroblasts were mixed in individual wells of a 96-well plate. In a blocking experiment, target cells were incubated with an anti-CD63 antibody (Novus, Clone H5C6, concentration 10 μg/ml) and/or macrophages were incubated with an anti-CD9 antibody (Abcam, #ab2215, Clone MEM-61, concentration 5 μg/ml. After a two hour incubation period on a shaker in a regular cell incubator, wells were washed with cold PBS, followed by trypsinization for 10 minutes. FACS buffer added, the plate was centrifuged, followed by another washing step with FACS buffer. After spinning down the plate, cells were incubated with a CD11b antibody (BioLegend) for 45 minutes on ice. Afterward, cells were washed and resuspended in FACS buffer, followed by flow analysis in a CytoFlex Flow cytometer (Beckman Coulter). For analysis, CD45+CD11b+ cells were gated and the percentage of RFP/PE+ cells determined. For immunofluorescence, macrophages were plated on fibronectin (MilliporeSigma) coated glass slides (VWR). After 45 minutes, fibroblasts were added and incubated with macrophages for one hour. After that, slides were vigorously washed three times with cold PBS, followed by a regular stain of cells plated on glass slides as described elsewhere in the method section.

Measuring phagocytosis in vivo. Cancer cells were subcutaneously injected, and tumors were harvested after 14 days. Upon euthanasia, the back skin was carefully detached from the underlying tissue and the tumors were visualized. Tumors were separated from the subcutaneous skin, minced with scissors and digested in 500 μl of DMEM supplemented with 40 μl of a 12.5 mg/ml liberase (Roche) solution for 30 minutes on a shaker at 37° C. The digestion reaction was quenched with DMEM/HPL medium, and the digestion mix and tissue pieces were filtered through a 70 μm filter (Falcon). Upon washing and spinning down the samples two times, cell suspensions were incubated with primary antibodies against CD11b and CD63 for 45 minutes on ice. Cell suspensions were washed with FACS buffer, resuspended in FACS buffer and analyzed either on a Aria Sorter (BD) or a Cytoflex (Beckman Coultier). The analysis was performed with FlowJo (FlowJo Inc).

Flow cytometry and Sorting. For live cells, we washed single cell suspensions with FACS buffer (PBS+2% FBS+1% Penicillin/Streptomycin+1 mM EDTA+25 mM HEPES) and then stained the cells with the primary antibodies for 45 minutes. Cells were washed and subsequently resuspended with FACS buffer. For intracellular stainings, cells were fixed with BD Wash/Perm (Becton, Dickinson and Company), followed by the same steps used for the live cells, with the exception of using BD Wash/Perm (Becton Dickinson and Company) instead of the FACS buffer. For flow cytometry, we either used the CytoFlex Flow Cytometer (Beckman Coulter) for analysis only, or the BD FACS Aria Ill (Becton, Dickinson and Company) for analysis and sorting. We performed the data analysis with FlowJo (FlowJo, LLC).

ELISA of CD63 in cell supernatant. To measure CD63 in the supernatant of lung cancer cells and lung fibroblasts, a Human CD63 ELISA Kit (Thermo Scientific) was used and performed according to the manufacturer's specifications. 20,000 cells were plated into the wells of a 24-well-plate in 200 μl of serum-free DMEM/F12 supplemented with PVA and Penicillin-Streptomycin. After 24 hours, the supernatant was harvested and frozen until the supernatant from all desired cultures was collected. Before running the assay, the supernatants were diluted with 100 μl of Diluent B and centrifuged at 14,000 g for 2 minutes. All steps and incubation periods were performed according to the specifications in the manual, including an initial 2.5 incubation in the supplied plate, followed by 1 hour incubation in the biotinylated antibody reagent, 45 minutes incubation in the Streptavidin-HRP solution and 30 minutes in the TMS substrate. Between each incubation step, the plate was washed four times with 300 μl of wash buffer. After incubating the plate with the TMS substrate, the reaction was stopped with the Stop solution and absorbances were measured with an Infinite plate reader (Tecan).

EdU Assay. EdU was measured with the Click-it EdU AF594 (or AF647) Kit (invitrogen). 10,000 cells were plated in the wells of a 24-well-plate in 500 μl of DMEM/HPL medium. After 24 hours, EdU (Invitrogen) was added directly to the wells. Cells were harvested after 45 and 90 minutes and fixed in 4% PFA (Electron Microscopy Service) for 15 minutes at room temperature. Cells were washed with PBS and incubated for 15 minutes in saponin-based wash and permeabilization buffer, followed by addition of the EdU reaction mix, another washing step with the wash and permeabilization buffer, and resuspension in wash and permeabilization buffer. Samples were analyzed on a Cytoflex (Beckman Coultier).

Transfection with sgRNA/Cas9 mRNA. We purchased modified sgRNA against CD63 from Synthego after using their knockout designer. Upon receival of the sgRNA, we diluted them in nuclease-free H2O. We performed a RNP transfection with the transit-mRNA transfection kit (Mirus) and Alt-R S.p. HiFi Cas9 Nuclease V3 (Integrated DNA Technologies). One day before the transfection, we plated 25,000 target cells into the wells of a 24-well plate in antibiotic-free DMEM medium with 10% FSCS. After warming up Optimem medium to room temperature (Gibco), 7.5 μg of sgRNA and 0.56 μl of Cas9 were added to 64 μl of Optimem, followed by mixing through pipetting up and down and an incubation of 30 minutes. Before reaching the end of the 30 minutes, 50 μl of DMEM were pipetted into 1.5 ml tubes, followed by adding first 1.5 μl of transit-mRNA (Mirus) and second 1.5 μl of mRNA Boost (Mirus). This mixture was vortexed, and 15 μl of the RNP mix were added to each tube. After shortly vortexing these mixtures, they were incubated for 2 minutes. Then, they were added drop-wisely onto the target cells, and thoroughly mixed with the medium through pipetting up and down. The cells were incubated for 2 days, before transferring the cells into the wells of 6-well plate or a 10 cm dish. For the purification of knockout cultures, we either used FACS for cancer cell cultures or MACS for fibroblasts.

Plasmid transfection. The CD63-eGFP plasmid was a gift from Paul Luzio and was purchased from addgene. Upon arrival, we plated the bacteria stab on kanamycin (MilliporeSigma) containing agar plates, followed by transferring single colonies into 14 ml tubes with LB medium with kanamycin. The next day, we transferred the bacteria-containing medium into flasks with 200 ml of LB medium with kanamycin. The next day, we performed a maxiprep (Qiagen) according to the manufacturer's specifications. The final plasmid was diluted in TE buffer. For transfecting lung fibroblasts, 50,000 fibroblasts were plated into the wells of a 6-well-plate. The next day, we mixed 6 μg of plasmid DNA with 18 μl of Fugene (Promega) in 300 μl of DMEM medium. After an incubation of 15 minutes, we added the mixture drop-wisely directly onto the cells. We incubated the cells with the plasmid for two days before proceeding to the next experiments.

Tissue fixation and hematoxylin staining. We kept harvested tissue in 10% formalin overnight. Tissue was then submitted to the Stanford Human Pathology/Histology Service Center for paraffin-embedding and cutting. We deparaffinized and rehydrated the tissue slides with xylene and a descending ethanol row. After washing the slides in PBS, we incubated them in hematoxylin (American MasterTech) for 4 minutes, then in bluing reagent (ThermoFisher Scientific) for 2 minutes and in Harleco® (MilliporeSigma) for 2 minutes. Slides were dehydrated with ethanol and xylene (MilliporeSigma) and mounted wit Permount® (ThermoFisher Scientific).

Immunofluorescence Staining of paraffin-embedded sections. When using paraffin-embedded tissue, we first deparaffinized and rehydrated the tissue. Then, we performed antigen retrieval with a citric acid buffer in a pressure cooker for 15 minutes, followed by blocking with 5% serum. Sections were incubated with the primary antibody over night at 4° C. After washing in PBST, we incubated the sections with the secondary antibody at room temperature for 30 minutes under agitation. Sections were washed, counterstained with DAPI and mounted with fluoromount (SouthernBiotech). Images of histological slides were obtained on a Leica Eclipse E400 microscope (Leica) equipped with a SPOT RT color digital camera model 2.1.1 (Diagnostic Instruments).

Immunofluorescence Staining of cells plated on glass slides. For plating cells on glass slides (VWR), areas were encircled on glass slides with a fat pen (Vector Laboratories), followed by sterilization for at least one hour under UV light. Then, fibronectin (20 μg/ml PBS) was added. After incubating the slides for one hour in a cell incubator at 37° C., the fibronectin solution was aspirated, and the slides were dried in the cell culture cabinet. Cells were added onto the glass slides and incubated for different amount of times in regular cell culture medium. Thereafter, the glass slides were washed in PBS, followed by fixing the cells with 4% PFA at room temperature for ten minutes. The slides were washed again in PBS, followed by permeabilization in TritonX for ten minutes for intranuclear stainings. Otherwise, cells were blocked with 5% serum for one hour. Afterwards, the staining procedure equaled the staining protocol of the paraffin-embedded slides.

Immunohistochemistry. When using paraffin-embedded tissue, we first deparaffinized and rehydrated the tissue. Then, we performed antigen retrieval with a citric acid buffer in a pressure cooker for 15 minutes, followed by blocking with 5% serum. Sections were incubated with the primary antibody over night at 4° C. After washing in PBST, we incubated the sections with the secondary HRP-antibody at room temperature for one 30 minutes under agitation. Sections were washed in PBST and ddH2O followed by incubation for 15-20 minutes in AEC Peroxidase Substrate (Vector Laboratories). Sections were washed in ddH2O and incubated in Modified Mayer's Hematoxylin for 4 Minutes. After washing in ddH2O, slides were mounted with fluoromount (SouthernBiotech).

Proximity-Ligation Assay. To determine physical proximity, we used a Duolink™ In Situ Red Starter Kit (MilliporeSigma). We plated target cells and macrophages on fibronectin-coated glass slides for one hour, before carefully washing the slide in PBS, followed by an incubation for 10 minutes in 4% PFA, another washing step in PBS, and 60 minutes incubation in PBS with 5% donkey serum. Then, the primary antibodies against CD63 (Abcam, clone TS63) and CD9 were resuspended in PBS with 1% donkey serum and incubated over night at 4° C. Single stain controls were used to determine specificity. The rest of the procedure was performed as outlined in the manufacturer's specifications. The next day, slides were washed with wash buffer, incubated for 1 hour with the PLA probes at 37° C., washed wish wash buffer, incubated for 30 minutes with the ligase at 37° C., washed with wash buffer A, incubated with the polymerase (amplification step) for 100 minutes at 370 C, followed by two washing steps in wash buffer B. Samples were mounted with Duolink In Situ Mounting Medium, covered with a cover glass and imaged with a confocal microscope.

Preparation of tissue microarrays. To prepare the tissue microarray, the Stanford Pathology database was searched for resected lung adenocarcinomas. Blocks and slides with H&E stains were requested and suitable areas of interest identified. For the TMA, cores were transferred from the original block into a paraffin block. The paraffin block was melted at 55° C. for one hours, before being cut.

Mass spectrometry analysis of sorted IPF lungs. Lung tissue from patients with idiopathic pulmonary fibrosis was minced and digested in CollagenaseIV for 30 minutes and filtered through a 70 μm cell strainer. Thereafter, cells were washed with 10 ml of PBS, centrifuged at 300 g for 5 minutes, washed with 10 ml of FACS buffer, and centrifuged again at 300 g for 5 minutes. Cells were stained for CD45 and Epcam for 45 minutes on ice. Afterwards, cells were washed twice with FACS buffer. DAPI was added to the cell suspensions, and the cell suspensions were sorted on a BD Aria Sorter for CD45+, Epcam+ and lineage-negative cells.

Sorted cells were pelleted and washed 2× with ice-cold PBS to remove any remaining FBS (1,300 rpm×5 min 4° C.). PBS was aspirated away, and the pellets were snap frozen with liquid nitrogen prior to storage at −80° C. Prior to lysis, cells were thawed on ice and subjected to sample preparation with the PreOmics iST NHS kit according to literature protocol. The only modification made to the protocol was scaling down in volume of lysis buffer and digest buffer (20 μL each). Samples were resuspended in 12 μL of LC-Load Buffer from the iST NHS kit and peptide concentration determined (Pierce Quantitative Colorimetric Peptide Assay).

A nanoElute was attached in line to a timsTOF Pro equipped with a CaptiveSpray Source (Bruker). Chromatography was conducted at 40° C. through a 25 cm reversed-phase Aurora Series C18 column (IonOpticks) at a constant flow-rate of 0.4 μL/min. Mobile phase A was 98/2/0.1% Water/MeCN/Formic Acid (v/v/v) and phase B was MeCN with 0.1% Formic Acid (v/v). During a 120 min method, peptides were separated by a 4-step linear gradient (0% to 15% B over 60 min, 15% to 23% B over 30 min, 23% to 35% B over 10 min, 35% to 80% over 10 min) followed by a 10 min isocratic flush at 80% for 10 min before washing and a return to low organic conditions. Experiments were run as data-dependent acquisitions with ion mobility activated in PASEF mode. MS and MS/MS spectra were collected with m/z X00 to 1500 and ions with z=+1 were excluded.

Raw data files were processed with Byonic software. Fixed modifications included +113.084 C. Variable modifications included Acetyl+42.010565 N-term, pyro-Glu-17.026549 N-term Q, pyro-Glu-18.010565 N-term E. Precursor tolerance 30.0 ppm.

Analysis of publicly available mass spectroscopy data. To validate our results from IHC, we performed additional analyses of publicly available mass spectroscopy data from 38 lung adenocarcinomas and matched control lung tissue. We downloaded the dataset “LC7 Proteomic Analysis of Matched Control and NSCLC Lung Adenocarcinoma Tissue” (MassIVE MSV000080825) from the homepage of the Center for Computational Mass Spectrometry at the University of California San Diego. We performed the analysis with the Knime Analytics Platform (Knime). A decoy database from a uniport database was created with Knime's “DecoyDatabase” node. Peaks were found with the PeakPickerHiRes and MSGFPlusAdaptor were used to find matches in a mass spec database. A false-discovery rate of 0.01 was applied. Each sample contributed three replicates. Replicates were analyzed separately. For the final analysis, the mean value of the three replicates was used.

Primary antibodies. Flow cytometry: CD11b (Biolegend, #301305, Clone ICRF44), CD31 (Biolegend, #102522, Clone MEC13.3), CD45 (Biolegend, #103109, Clone 30-F11), CD63 (Biolegend, #143906, Clone NVG-2), CD63 (Biolegend, #353007, Clone H5C6), CD326 (Biolegend, #118217, Clone G8.8)

MACS Sorting: CD63 (Biolegend, #353017, Clone H5C6). Immunostainings: CD9 (Abcam, #ab92726), CD63 (Abcam, #ab59479, Clone TS63), CD63 (Abcam, #ab1318, Clone NK1/C3), CD63 (R&D, #MAB5417), CD81 (Abcam, #ab128202), CD151 (Abcam, #ab33315, Clone 11G5a), FSP1 (abcam, #ab58597), FSP1 (MilliporeSigma, #07-2274), GFP (Abcam, #ab183734), Ki67 (Abcam, #ab15580)

Secondary antibodies. Immunostainings: AF488 donkey anti-mouse (Invitrogen, #A-21202), AF488 goat anti-rabbit (CST, #44125) AF594 donkey anti-goat (Novus, #NBP1-75607), AF594 goat anti-mouse (Invitrogen, #R37121), AF594 goat anti-rat (Invitrogen, #A-11007), HRP goat anti-rabbit (Abcam, #ab205718), HRP donkey anti-mouse (Abcam, #ab6820),

Statistics. We used Graphpad prism (Graphpad Software Inc) for creating graphs and running statistical analyses. When two values were compared, a two-sided student's t-test was used, if more than two values were analyzed, we used ANOVA tests with corrections for multiple comparisons. Experiments included at least three independent values. Hypotheses were tested through at least two independent experiment. We regarded experiments with different cell lines as independent experiments. P values below 0.05 were considered as statistically significant.

### Example 2

Lung cancer remains a disease with poor survival. Non-small cell lung cancer (NSCLC) comprises squamous cell carcinomas and adenocarcinomas, and as smoking rates drop in many countries, adenocarcinomas become the predominant entity. Many adenocarcinomas show a desmoplastic reaction with a pronounced fibrotic stroma component. The tumor tissue microenvironment includes cancer cells, other epithelial cells, such as endothelial cells, immune cells, and fibroblasts—also called cancer-associated fibroblasts (CAF). CAF contribute to lung cancer progression by secreting a variety of pro-tumorigenic cytokines. Though the immune system also secretes pro-tumorigenic cytokines, it has the potential to effectively eliminate target cells. Immunotherapies against PD1/PDL1 and CTLA4—unleashing the adaptive immune system—have improved cancer therapies, including lung cancer. Though the innate immune system has received less attention, the CD47 antibody magrolimab is currently being investigated in a phase Ill study for myelodysplastic syndrome, a form of blood cancer. As a macrophage immune checkpoint and “don't-eat-me-signal” it protects target cells against phagocytosis through macrophages. Most lung cancers are diagnosed in advanced stages in which complete surgical tumor resection is not feasible and true cancer cure—defined as the absence of detectable tumor cells—unlikely. However, new therapies either as small-molecule inhibitors or immunotherapies have also brought major therapeutic improvements. Unfortunately, only a minority benefits from these therapies as their cancers are initially resistant or grow resistant over time. Therefore, the array of available therapies targeting different signaling pathways needs to be expanded. Thereby, lung cancer could be turned into a chronic disorder for which patients can switch between therapies multiple times. The majority of new cancer therapies are antibodies due to their specificity targeting surface proteins. Groups of surface proteins are for example the immunoglobulin superfamily, the alpha/beta hydrolase superfamily, or tetraspanins. Tetraspanins include 29 proteins that all incorporate 4 transmembrane domains. Tetraspanin family members CD9, CD63, and CD81 are part of intra- and extracellular vesicles and commonly serves as markers for exosomes. As a prognostic factor in various cancers, tetraspanins led to mixed results. While increased CD63 expression in colorectal cancer correlated with poor survival, it correlated with improved survival in gastric or esophageal cancer.

Here, we investigated whether tetraspanins drive tumor progression and whether tetraspanins could serve as new therapeutic target for lung cancer. For this purpose, we would analyze clinical patient samples and validate the effect of the specific tetraspanin in vitro and in vivo.

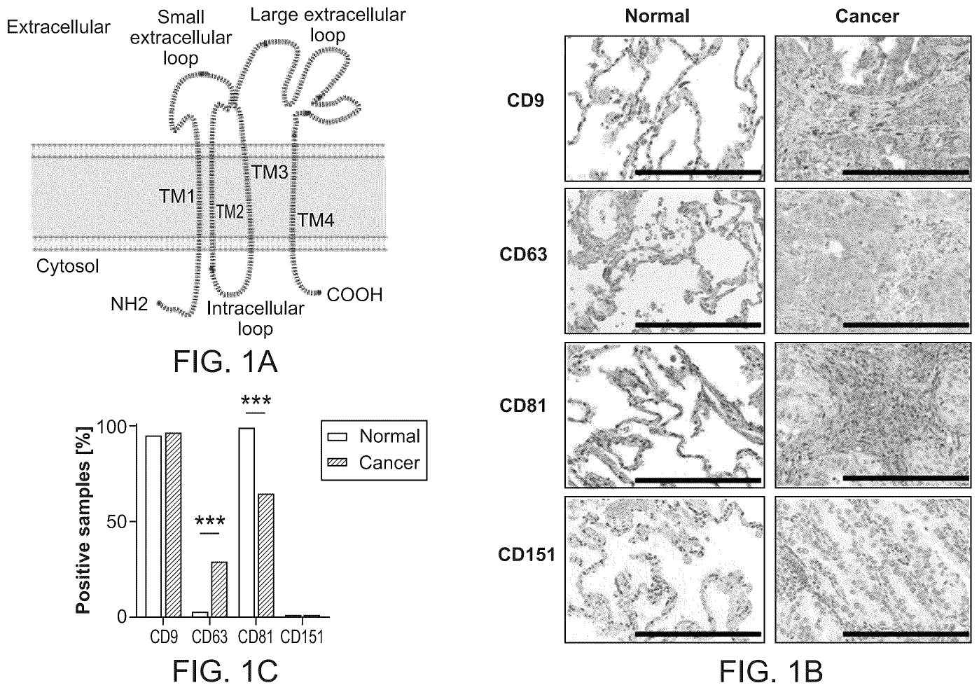

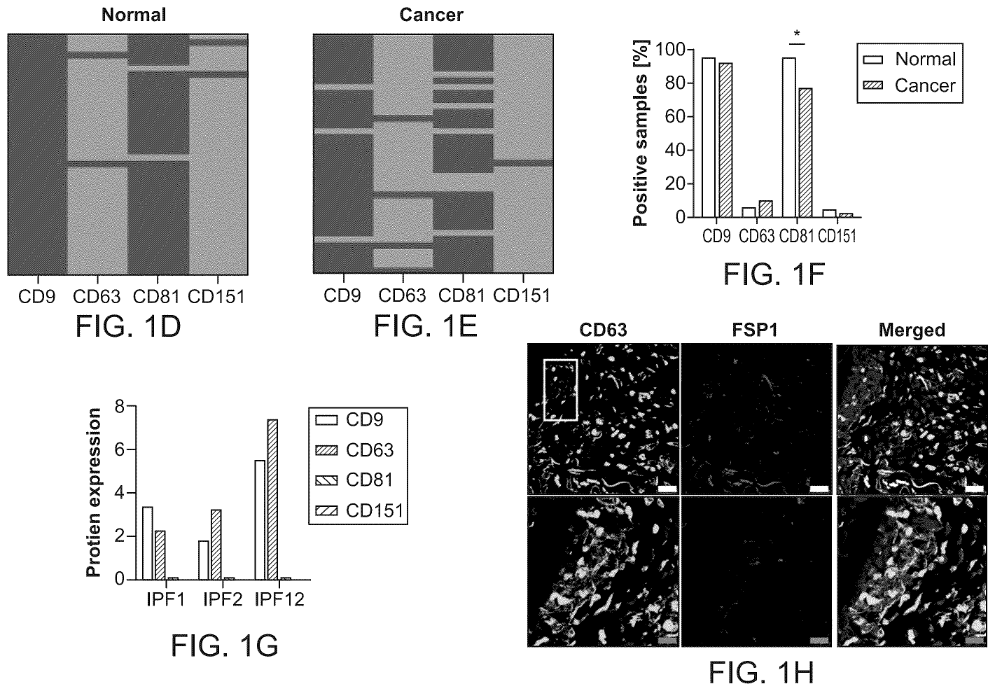

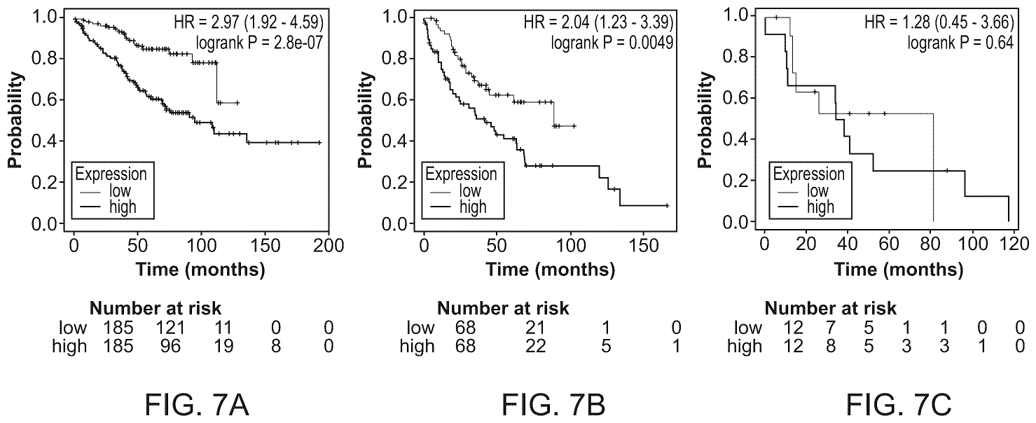

Lung cancer upregulates CD63 at the gene and protein expression level. To study the expression of tetraspanins, a tissue microarray (TMA) was first built that contained tissue cores from patients with lung adenocarcinomas and matched normal lung tissue (FIG. 1 A). As tetraspanins include more than 30 family members, the four tetraspanins CD9, CD63, CD81, and CD151 were stained for (FIG. 1 B). While CD9 and CD81 was more strongly expressed in normal lung and lung cancer tissue, CD63 was the only tetraspanin with a stronger expression in cancer tissue than normal tissue (FIG. 1 C). For this reason, the analysis of CD63 was continued, and determined CD63 gene expression on the TMA through in situ hybridization (ISH). Confirming the IHC results, CD63 was more expressed in both lung cancer cells and tumor stroma than in normal lung tissue (FIG. 1 C-G). Additionally, higher CD63 expression was correlated with worse survival in a publicly available dataset (FIG. 1 H, FIG. 7).

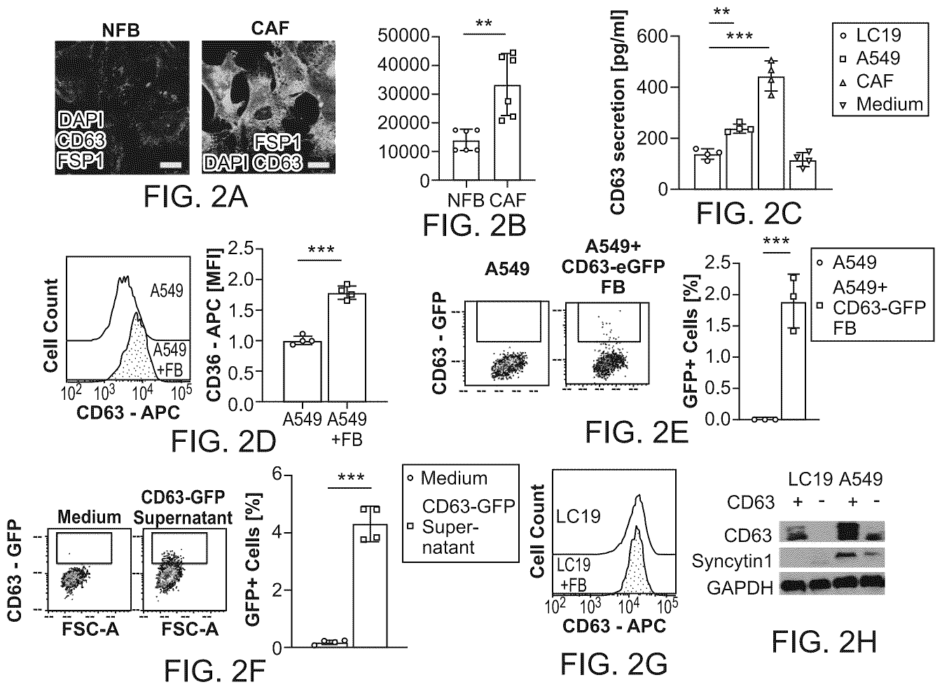

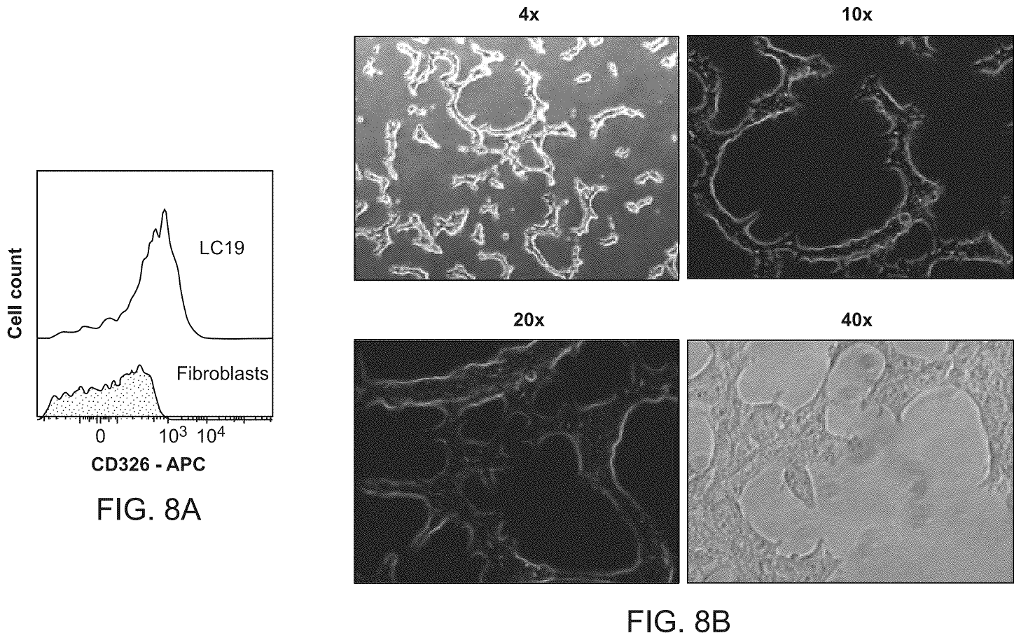

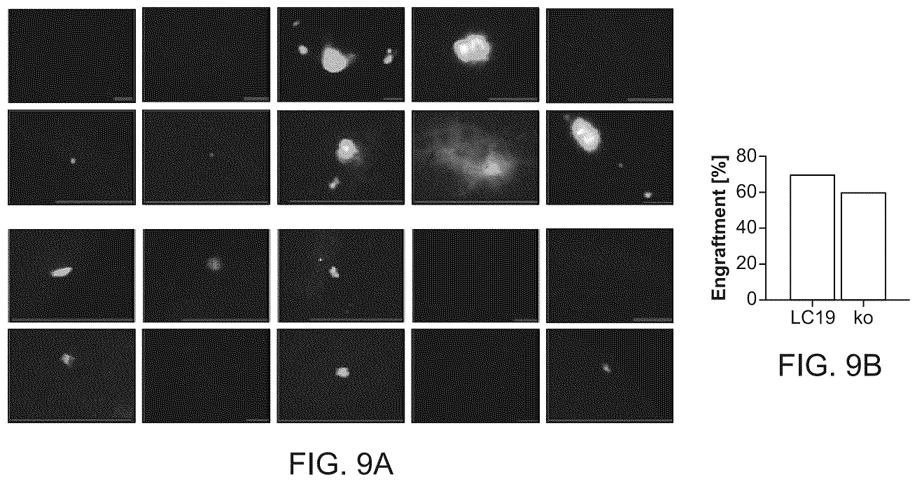

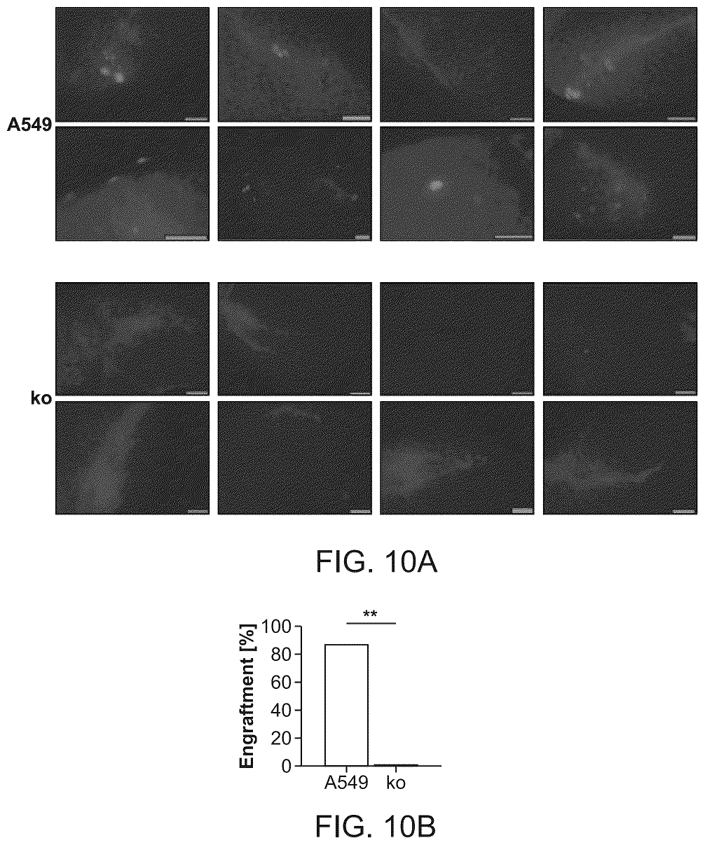

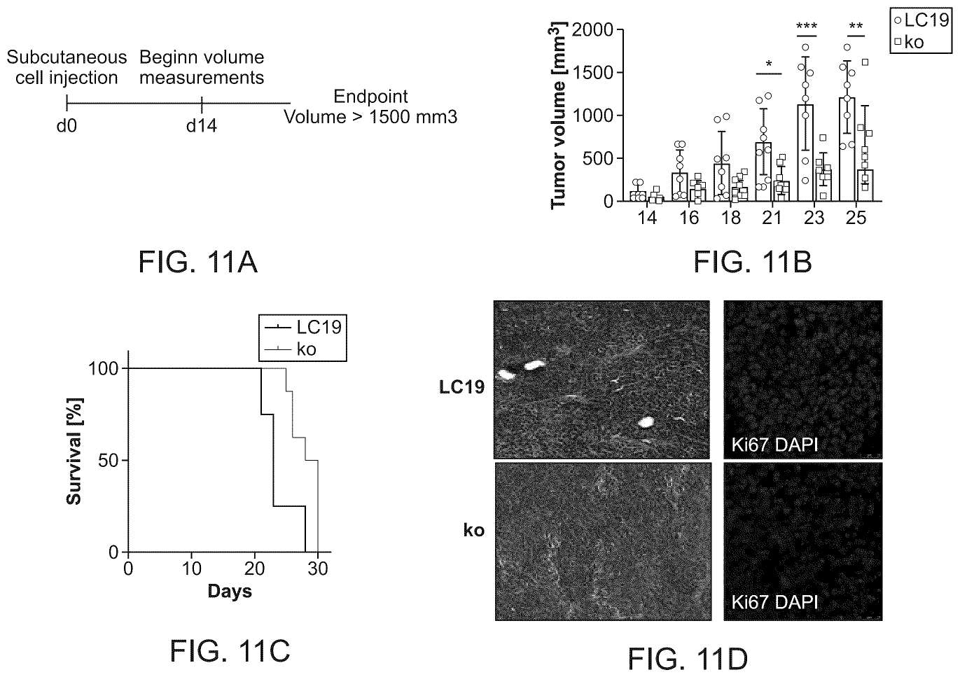

Exosomes transport CD63 from fibroblasts to lung cancer cells via Syncytin1. Next, the expression of tetraspanins in in vitro cultures was measured with immunofluorescence (IF) and Flow Cytometry (FC). Two established lung cancer cell lines were used (A549 and H810) and generated one primary lung cancer cell line from a patient with NSCLC (FIG. 8). Additionally, primary lung fibroblast cultures from normal lung (NFB) were established, and lung cancer (CAF). Both in IF and FC, CAF expressed more CD63 than NFB (FIG. 2 A, B). Tetraspanins are found in intra- and extracellular vesicles. For this reason, it was hypothesized that lung fibroblasts secrete CD63 and that the secreted CD63 is incorporated into the plasma membranes of cancer cells. In accordance with this hypothesis, CAF secreted CD63 into the supernatant in vitro. Next, co-culturing A549 cells with fibroblasts increased CD63 expression in the A549 cells (FIG. 2 C-E). The fibroblasts were then transfected with a plasmid containing GFP-labeled CD63. A549 cells were co-cultured either directly with these fibroblasts or with the supernatant of these fibroblasts. In both cases, GFP was detected on the membranes of the A549 cells through flow cytometry (FIG. 2 F-G). While co-culturing with fibroblasts increased CD63 in A549 cells, it did not increase CD63 expression in LC19 cells. Looking for an explanation, A549 and LC19 cells were further analyzed through WB. Here, it was found that A549 cells but not LC19 cells expressed Syncytin1 that serves as a receptor for extracellular vesicles (FIG. 2 H).

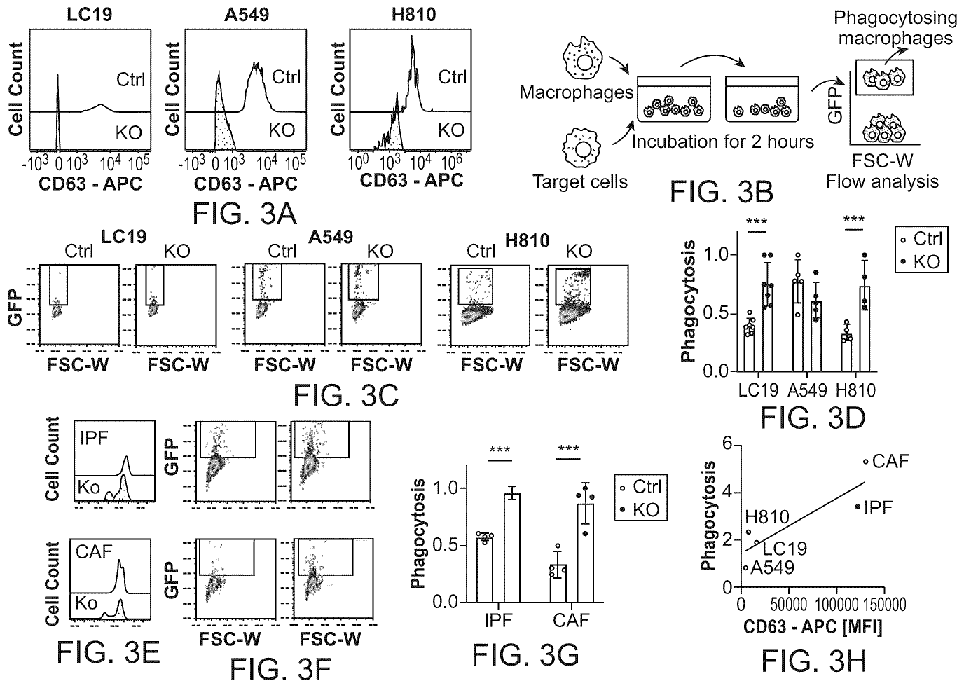

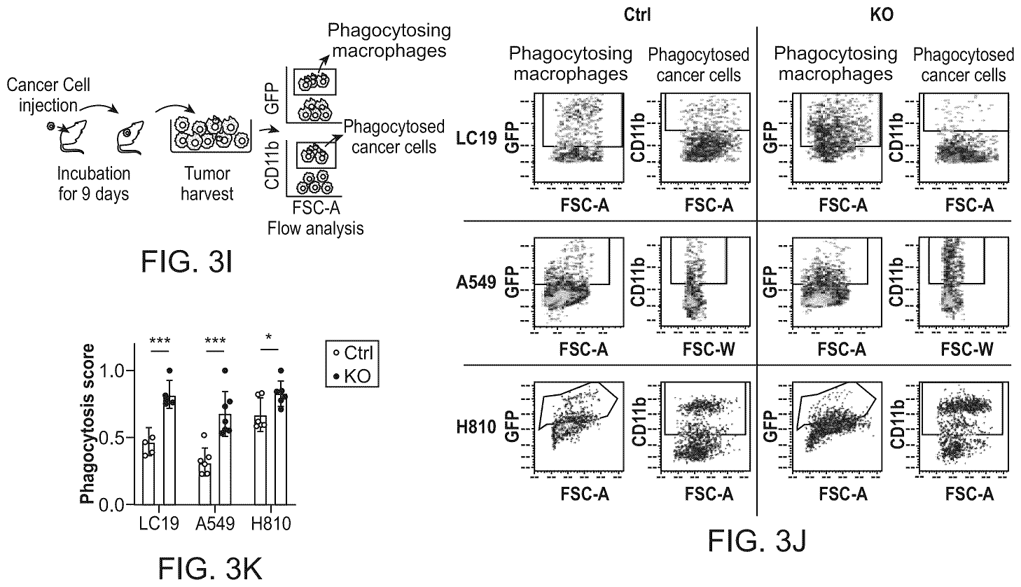

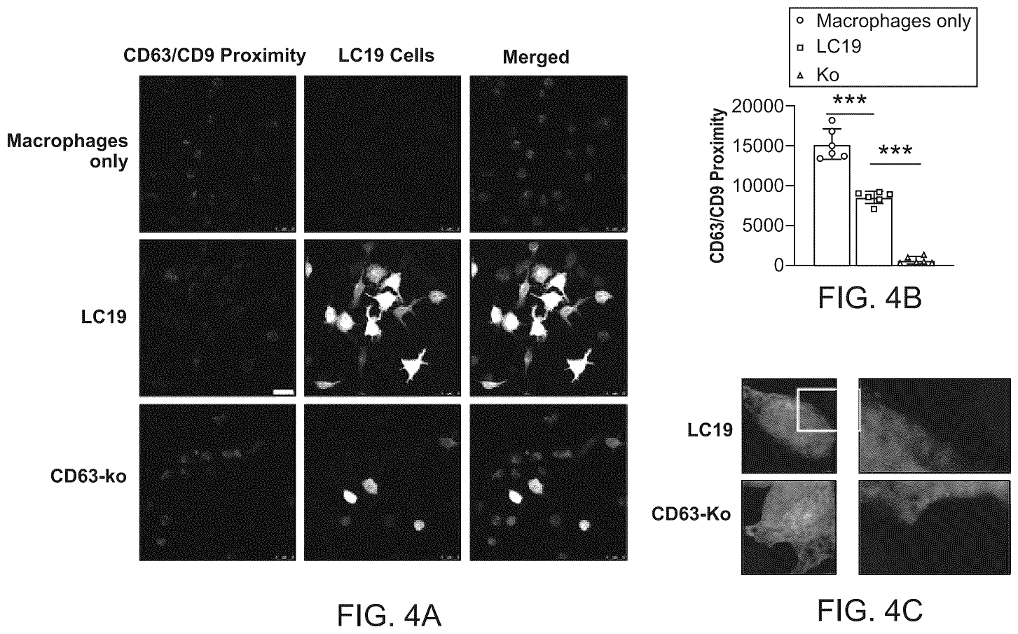

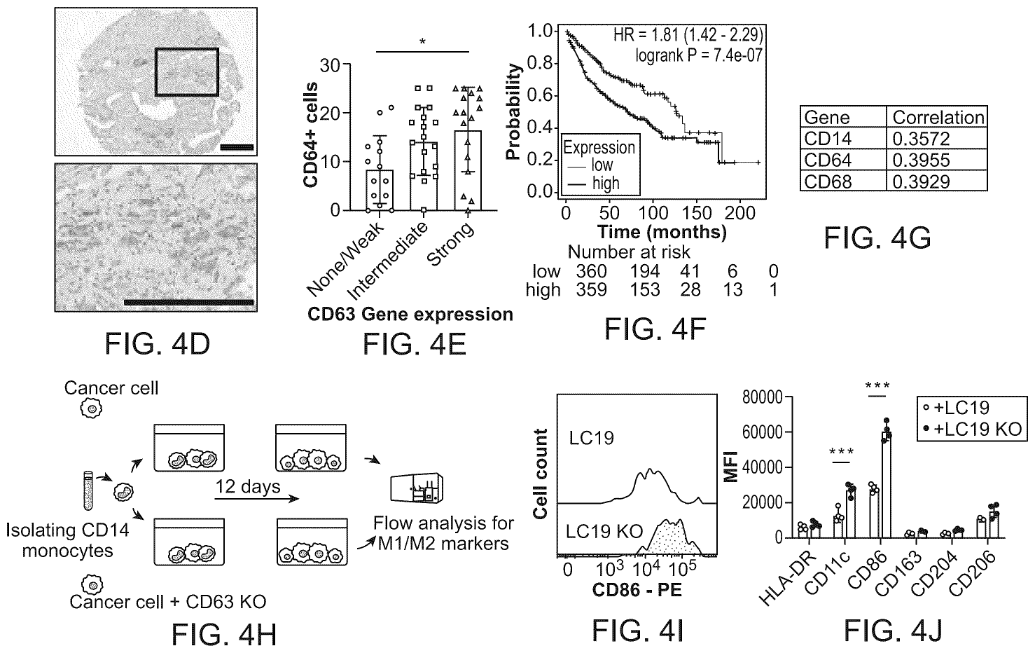

CD63 serves as a “don't-eat-me-signal” for lung cancer cells and lung fibroblasts. Next, how CD63 interacts with macrophages as part of innate immune system was examined. For this purpose, CD63 in lung cancer cells was deleted and ran in vitro phagocytosis assays with primary peripheral blood derived human macrophages. In the in vitro phagocytosis assays, GFP-labeled lung cancer cells were incubated with the macrophages for 2 hours before the uptake of GFP-positive cellular material into the macrophages was measured with flow cytometry (FIGS. 3A-B). It was discovered that deleting CD63 increased phagocytosis of LC19 and H810 cells (FIGS. 3C-D). Then, the in vitro phagocytosis assays were repeated with lung cancer fibroblasts. As the primary fibroblasts were unable to survive FACS, the knockout population was purified through MACS. Though MACS only led to an incomplete purification of the knockout cell populations, the reduction of CD63 in lung cancer fibroblasts increased phagocytosis (FIG. E-G). When compensating for the incomplete purification in the fibroblasts, plotting the increase in phagocytosis against the expression of CD63 for lung cancer cells and lung fibroblasts demonstrated a correlation between a higher increase in phagocytosis and a higher CD63 expression (FIG. H). After the in vitro phagocytosis assays, phagocytosis was measured in vivo. For this purpose, GFP-labeled lung cancer cells was injected subcutaneously into immunocompromised mice. After nine days, the transplanted cells were harvested for flow cytometry. As the percentage of GFP+ macrophages depended on the infiltration of the tumor with macrophages, both the percentage of GFP+ macrophages and the percentage of phagocytosed cancer cells were measured. Both numbers were combined to calculate a phagocytosis index. Thereby, deleting CD63 increased phagocytosis in all three lung cancer cell lines (FIGS. 3I-K). Looking for a receptor for CD63 on macrophages, a proximity ligation assay demonstrated an interaction between CD63 and CD9 (FIGS. 4A-C). On the lung cancer TMA, lung cancer samples with a higher CD63 gene expression showed an increased infiltrate with CD64+ macrophages. Additionally, higher CD64 expression in lung adenocarcinomas was correlated with poorer survival and CD63 positively correlated the three macrophage/monocyte-associated genes CD14, CD64, and CD68 (FIGS. 4D-G). In a monocyte differentiation, incubating bead-purified CD14+ monocytes with LC19 cells after CD63 KO led to the upregulation of the M1-associated proteins CD11c and CD86 compared to the incubation with LC19 cells without CD63 KO (FIGS. 4H-J).

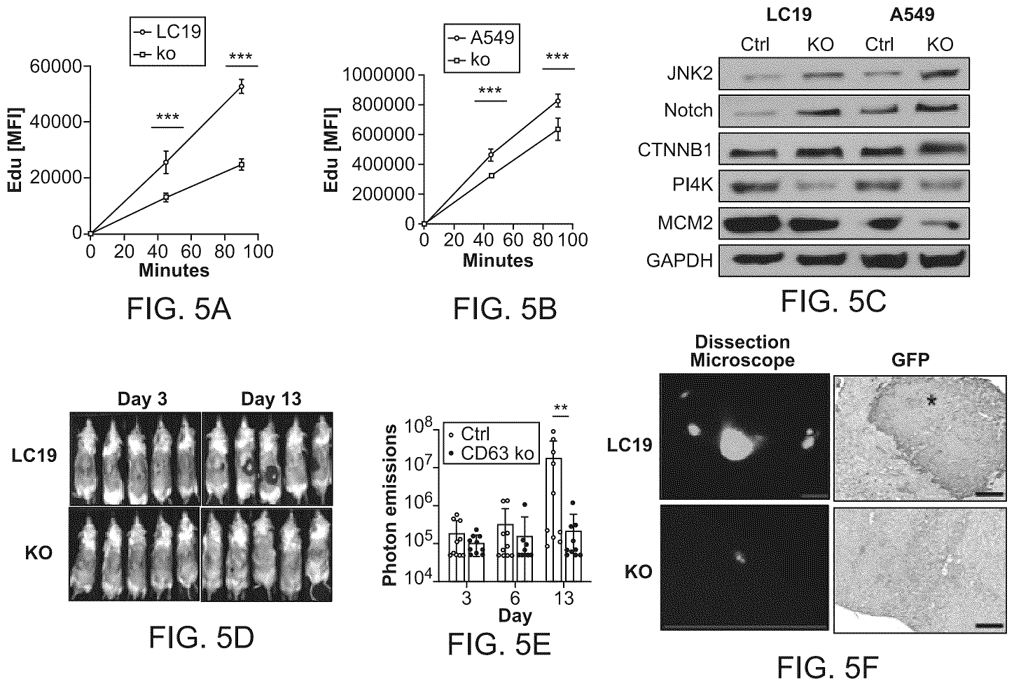

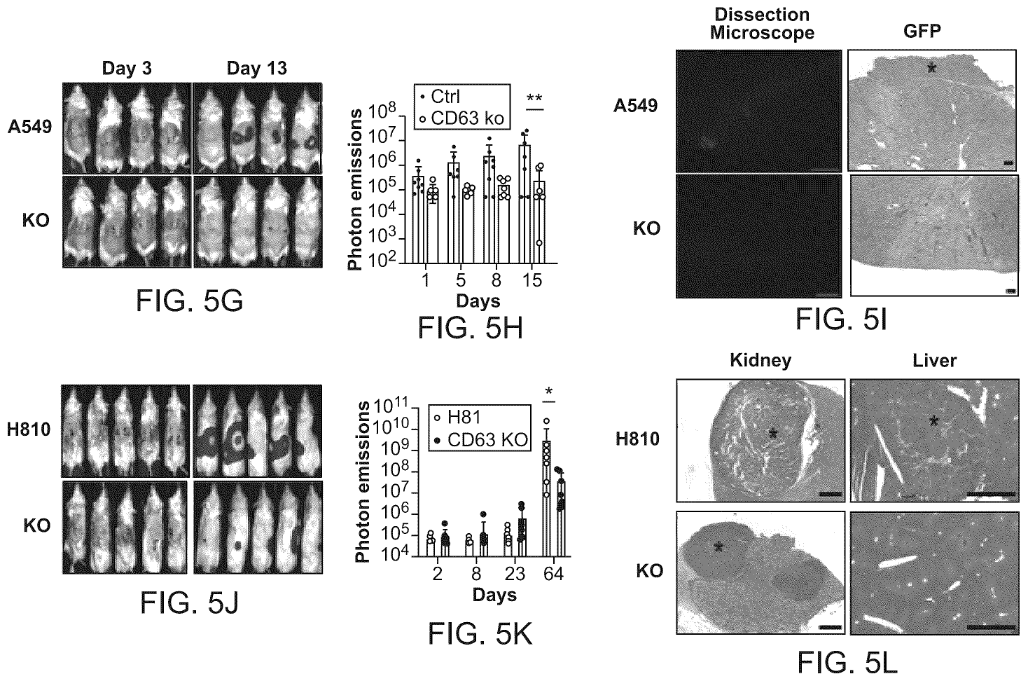

CD63 mediates cancer stem cell characteristics to lung cancer cells. Next, how deleting CD63 affects typical cancer-related characteristics was investigated. After demonstrating that CD63 deletion decreases proliferation, a WB indicated increased JNK2 and Notch expressions—markers related to both pro- and anti-tumorigenic potential. Additionally, it decreased PI4K expressions (FIGS. 5A-C). Next, an adaptive transfer model was used to test whether CD63 affects the tumorigenic potential in vivo. 1000-2000 GFP- and luciferase-labeled lung cancer cells were transplanted under the kidney capsule of immunocompromised mice and regularly tracked cell growth through optical imaging. While tumor growth after deleting CD63 was either prevented or strongly impaired, control tumors of LC19 and A549 grew fast within a couple of weeks. In contrast, H810 cells grew more slowly and tumor formed both in controls and after deleting CD63 after 60 days (FIGS. 5D-K). However, in the controls, H810 formed liver metastases while no liver metastases were found after deleting CD63 (FIG. 4L).

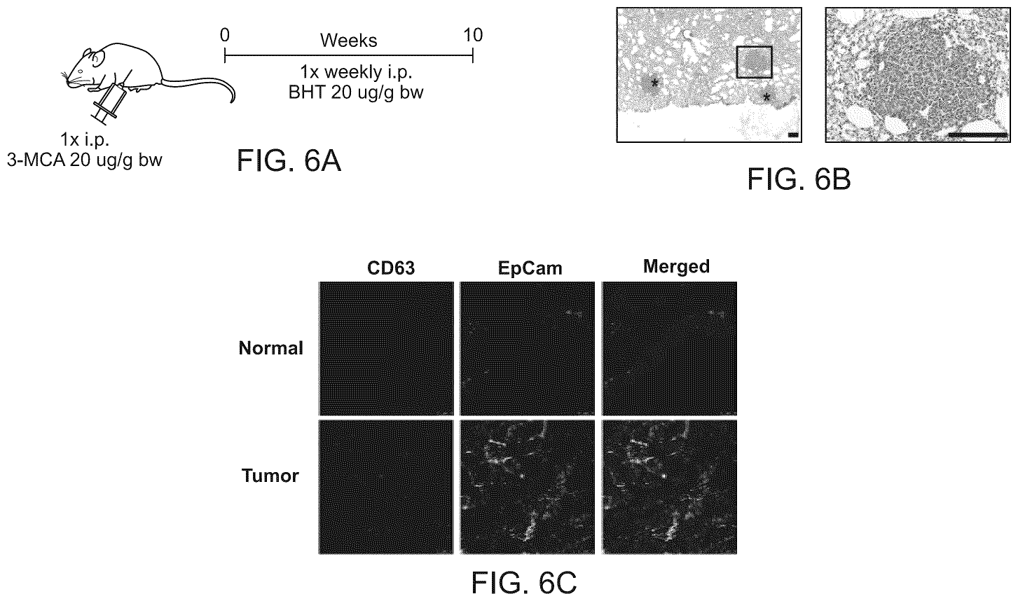

CD63 increases in epithelial cells and fibroblasts in a mouse lung tumor model. Finally, whether CD63 is also increased in a mouse model of lung fibrosis was investigated. Here, lung fibroblasts through Bleomycin inhalation were induced, followed by pulmonary JUN induction through doxycycline-administration. After 12 days, the lung tissue was harvested and analyzed the lung tissue through IHC and flow cytometry. Thereby, both methods demonstrated increased CD63 expression both in epithelial cells and fibroblasts. Next, lung tumor formation was induced through administration of 3-MCA and BHT over ten weeks and IHC confirmed CD63 expression in lung adenomas (FIG. 6A-C)

### Discussion

Lung cancer remains the primary reason for cancer-related mortality worldwide. As smoking rates decrease, lung adenocarcinomas become the dominating histological entity. Many adenocarcinomas share a desmoplastic reaction in which a fibrotic tumor stroma forms a substantial part of the tumor. The tissue microenvironment in lung cancer is built by cancer cells, other epithelial cells, immune cells, and fibroblasts. Thereby, cancer cells and fibroblasts synergistically drive tumor progression. The group of tetraspanin proteins shares four typical transmembrane domains and includes more than 30 family members. They are commonly found in intra- and extracellular vesicles. In cancer studies, tetraspanins often showed conflicting results, and individual tetraspanins are correlated with better survival in one cancer and worse survival in another type of cancer. Unfortunately, these studies often investigated only the correlation between a tetraspanin and survival without investigating the mechanism how a tetraspanin affects cancer progression. In this study, a comprehensive analysis of a tetraspanin with increased expression in lung cancer and its mechanism to drive tumor progression was preformed.

Patient samples were first analyzed for the protein expression of the tetraspanins CD9, CD63, CD81, and CD151. Thereby, CD63 was the only one with an increased expression of CD63 in the cancer compared to the matched normal lung tissue. ISH then confirmed increased CD63 expression in cancer cells and cancer stroma. Interestingly, increased CD63 expression was correlated with worse survival in a publicly available database, despite predominantly early-stage lung cancers were included.

In lung cancer, cancer cells interact with CAF and immune cells during tumor development and progression. In the study, it was identified that CD63 on fibroblasts, e.g. as extracellular vesicles, can be merged into the cell membrane of cancer cells. Thereby, the fusion is dependent on the cell-fusion protein syncytin1, and no increase in CD63 occurred in syncytin1-negative LC19 cells.

Cancers develop mechanisms to evade immune surveillance. Surface CD63, either transported from the cell inside or merged into the cell membrane from the outside, regulated macrophage-mediated phagocytosis, and deleting or blocking CD63 increased phagocytosis both in vivo and in vitro in lung cancer cells and lung fibroblasts. Supporting the interaction between CD63 and phagocytosis, a higher initial CD63 expression led to a higher increase in phagocytosis after CD63 deletion. A proximity ligation assay showed the physical connection between CD63 on cancer cells, and CD9 on macrophages, though this interaction might not be exclusive, as other CD63 ligands are known such as CD82 or CD117, both found on macrophages as well. So far, the results suggested an adverse effect of CD63 on macrophage functions. Though macrophages can eliminate cancer cells, they can also drive tumor progression through the secretion of various immune suppressive cytokines. The M1/M2 concept—though imperfect—states that macrophages differentiate either towards the M1 phenotype that is associated with better survival in cancer or towards the pro-tumorigenic M2 phenotype. Tumor-associated macrophages (TAM) are of the M2 phenotype and exhibit a tumor-suppressive function. In accordance with an adverse effect of CD63 on macrophage function, higher CD63 expression in patient samples was associated with a higher infiltration with CD64+ monocytes and macrophages. Additionally, deleting CD63 in LC19 led to an increase in M1 markers, indicating the CD63 moves macrophages towards the M2 fate.

Tumor progression not only relies on the interaction between the cancer cell and its surroundings, but also on cancer-cell intrinsic mechanisms. Cancer stem cells (CSC) have an increased capacity of self-renewal. Some of the proposed CSC markers exhibit opposite effects. For example, JNK2 is both associated with increased stemness and compensatory apoptosis and induced senescence. Similarly, while the Notch pathway plays an important role in cancer stem cell maintenance, Notch also serves as a tumor suppressor and is commonly downregulated in a variety of cancers. In the study, deleting CD63 impaired tumor engraftment in an adaptive transfer model. As tumor engraftment is a characteristic of cancer stem cells, it was assumed that the increased expression of Notch and JNK2 after deleting CD63 hints at the tumor-suppressive function both genes in the CD63 context. Finally, adenomas in a MCA-induced lung tumor model in mice expressed more CD63 compared to normal bronchial epithelia, indicating that CD63 increases in tumor development in mice as well.

Finally, research also aims at developing new therapies to reach better clinical outcomes. Immunotherapies, like PD1/PDL1 or CTLA4 blockade, or small-molecule inhibitors, like crizotinib, have prolonged survival for patients with lung adenocarcinomas over the recent years. Despite their success, many patients do not respond to these therapies or progress with time. Cure from lung cancer, as defined as absence of tumor cells, depends on early diagnosis and complete tumor resection. Unfortunately, most patients with lung cancer are diagnosed in advanced stages in which complete tumor resection is no longer feasible—a fact that is unlikely to change. However, similar to other cancers such as prostate cancer, new therapies can significantly prolong survival and finally turn lung cancer into a chronic disease in most cases. To reach this objective, an array of different therapies needs to be available to offer patients the possibility to switch between drugs multiple times. Today, immunotherapy heavily relies on boosting the adaptive immune system. Targeting the innate immune system—such as reestablishing and stimulating cancer cell elimination by macrophages—offers a new therapeutic axis. Until now, only magrolimab that targets the macrophage immune checkpoint CD47 has reached advanced clinical stages for hematopoietic cancers. In this study, several mechanisms were identified through which CD63 stimulates lung cancer survival and progression. As a surface protein, CD63 can be targeted by antibodies. As CD63 undergoes significant posttranslational modifications, the efficiency of a CD63 blockade likely depends on the specific modification. More work is required to identify this specific modification and epitope whose blockade offers the strongest clinical benefit. Nevertheless, the study conceptually demonstrates that blocking CD63 offers potential therapeutic benefits.

Our observation in patient samples that CD63 protein and gene expression is increased in lung cancer and that increased CD63 expression is correlated with poorer survival, contradicts a study by Kwon et al. in 2006 in which the authors observed decreased CD63 gene expression in lung cancer tissue compared to normal lung tissue and in which a higher CD63 expression in IHC stains was correlated with improved survival. Possible explanations for these differences are the use of qt-PCR on whole tissue lysates to determine gene expression by Kwon et al. and a bias towards advanced stages in samples with negative CD63 expression. In contrast, using ISH allowed us to determine CD63 gene expression with spatial resolution.

In conclusion, the study demonstrates new roles of CD63 in lung cancer. CD63 is transferred from fibroblasts in the tumor stroma to lung cancer cells and contributes to immune evasion by impairing macrophage-dependent phagocytosis and moving macrophages towards the tumor-suppressive M2 phenotype. CD63 maintains CSC-associated feature in lung cancer cells and its deletion impairs the engraftment of lung cancer cells in mice and upregulates tumor suppressors. Though more research is needed on how specific epitopes of CD63 contribute to these observations, the study shows how blocking CD63 could be a therapy for lung cancer.

### Methods

Husbandry. JUN mice were kept on a regular diet in the facilities of the Veterinary Service Center at Stanford University and had a B6/129 background. Nod.Scid.Gamma and Balb/cJ mice were purchased from the Jackson Laboratory. Mice were kept on a standard diet. Female and male mice were used. When different sexes were used for individual experiments, groups were sex-matched. Mice were not backcrossed and between 6 and 12 weeks of age.

Genotyping. To determine the genotype, tissue from the tail of newborn mice was harvested on day 10. The DNA with Quickextract (Lucigen Corporation) was digested at 68° C. for 90 minutes, followed by heat inactivation at 98° C. for five minutes. The genotyping PCR was ran for the Rosa26 and the collagen status with the Phusion® High Fidelity DNA Polymerase (New England Biolabs) and the same primers as described previously.

Adoptive transfer under the kidney capsule. After anesthetizing mice, areas over both flanks were shaved. After creating a flank cut the subcutaneous tissue was bluntly removed from the underlying soft tissue. The abdominal wall was incised and the kidney luxated out of the abdominal cavity. After piercing the kidney and detaching the renal capsule from the renal tissue, 1,000-2000 cells suspended in Matrigel (MilliporeSigma) were injected under the kidney capsule. Afterwards, the kidney was replaced into the abdominal cavity and the abdominal wall and the skin were separately sutured. Upon the endpoint, mice were euthanatized. The kidneys were excised and visualized under a dissection microscope.

Subcutaneous tumor cell injection. 400,000 cells were resuspended in 100 μl of DMEM and 100 μl of Matrigel (MilliporeSigma). Mice were anesthetized, the back was disinfected, and the cell suspension was slowly subcutaneously injected on both sides of the back. Afterwards, mice were put back into their original cage.

Luciferase-based optimal imaging. 100 μl of luciferin substrate (15 mg/ml) (Biosynth) was intraperitoneally injected. 15 minutes later, optical imaging was performed with the Lago optical imaging system (Spectral imaging instruments) and analyzed the images with the Aura Software from the same manufacturer.

Lung fibrosis induction in mice. To induce lung fibrosis in JUN inducible mice, 100 μl of bleomycin was administered through inhalation under anesthesia. 3 and 7 days thereafter, doxycycline (2 mg/ml) (MilliporeSigma) was administered through inhalation as well. On day 12, mice were euthanatized and the tissue was processed as described elsewhere in the methods.

Lung tumor induction in mice through 3-Methylcholanthrene and Butylated Hydroxytoluene. Mice received intraperitoneal 3-MCA (MilliporeSigma) (15 μg/g body weight, diluted in 500 μl of corn oil). Over the next ten weeks, mice received BHT (MilliporeSigma) intraperitoneally (20 μg/g body weight, diluted in 400 μl of corn oil (MilliporeSigma)) once weekly. Mice were then euthanatized and lungs were processed as described elsewhere in the methods.

Cell culture maintenance. All cell cultures were kept in DMEM (Caisson Labs) supplemented with 5% HPL and 1% Penicillin-Streptomycin (MilliporeSigma) in an incubator at 37° C. 95% O2/5% CO2. Cells were regularly checked for signs for infection and split once being more than 80% confluent. For splitting, cell cultures were washed with PBS and incubated with Trypsin (Gibco) for 5 minutes. The reaction was then quenched with DMEM+HPL. Cell suspensions were spun down and reapplied into cell dishes. For assays in which CD63 was determined either in the supernatant or on cell surfaces, serum-free DMEM/F12 (Gibco) medium supplement with 1% BSA (MilliporeSigma), 1% Penicillin-Streptomycin (MilliporeSigma), 10 mM HEPES (Fisher Scientific), 1 μM insulin (MilliporeSigma), and 1 μM transferrin (MilliporeSigma) was used.

Preparation of human platelet lysate (HPL) and HPL-containing medium. Expired human platelets were obtained from the Stanford Blood Bank. Then, platelets were lysed through five quick freeze-thaw cycles. Platelet lysates were then spun down at 4,000 g for 10 minutes, aliquoted into 15 ml tubes and stored at −80° C. For preparing cell mediums, platelet lysates were warmed up, then spun down at 4,000 g for 10 minutes, and sequentially filtered through 0.80, 0.45 and 0.22 μm filters. The final medium, containing DMEM, 5% HPL, 1% Penicillin/Streptomycin and 2 Units of Heparin/ml, was then filtered through a 0.22 μm filter and stored at 4° C.

Harvesting of mouse lungs for flow cytometry. Mice were euthanized with CO2. The abdomen and chest were disinfected with 70% EtOH, followed by incising the skin from the lower abdomen up to the neck. The thorax was opened, and the heart was flushed with 5 ml of 10 mM EDTA. The lungs were excised, minced with scissors, and filtered through a 70 μm cell strainer (Falcon). Cells were then used for flow cytometry.

Isolation of primary pulmonary fibroblast and lung cancer cell cultures. Human fibroblasts were obtained discarded fresh lungs tissues from de-identified patients. The tissue was minced with scissors, washed two times with PBS and then plated into a 10 cm cell culture dish in regular cell culture medium (see cell culture maintenance), supplemented with Ciprofloxacin (10 μg/ml) (Bioworld).

Generating human macrophage cultures. Blood was donated by one of the authors. After disinfecting the elbow, blood was drawn into EDTA S-Monovettes® (Sarstedt) with a 21G Vacutainer (BD) connected through a Multi Adapter (Sarstedt). Thereafter, 8 ml of blood was mixed with 32 ml of PBS. Then, the mixture was carefully applied onto 10 ml of Ficoll-Paque Plus (GE) and spun for 30 minutes at room temperature with 400 g with low acceleration and no break. Afterwards, the buffy coat was transferred into a 50 ml tube with transfer pipettes and washed with PBS. After a centrifugation step, cells were plated into 10 cm Petri dish (Corning) in 6 ml of regular DMEM/HPL Medium. After two hours, the medium was replaced by 8 ml DMEM/HPL medium supplemented with 20 ng/ml M-CSF (Gibco). The medium was exchanged on days 3, 5, and 7. The macrophages were used for phagocytosis assays 7 to 9 days after their harvest.

Monocyte differentiation assay. To test how monocytes differentiate over time in vitro in co-incubation with lung cancer cells, CD14+ monocytes were purified with the EasySep™ Human Monocyte

Phagocytosis assay in vitro. For measuring phagocytosis through flow cytometry, 10,000 to 25,000 macrophages and fibroblasts were mixed in individual wells of a 96-well plate. In a blocking experiment, target cells were incubated with an anti-CD63 antibody (Novus, Clone H5C6, concentration 10 μg/ml) and/or macrophages were incubated with an anti-CD9 antibody (Abcam, #ab2215, Clone MEM-61, concentration 5 μg/ml). After a two hour incubation period on a shaker in a regular cell incubator, wells were washed with cold PBS, followed by trypsinization for 10 minutes. FACS buffer added, the plate was centrifuged, followed by another washing step with FACS buffer. After spinning down the plate, cells were incubated with a CD11b antibody (BioLegend) for 45 minutes on ice. Afterward, cells were washed and resuspended in FACS buffer, followed by flow analysis in a CytoFlex Flow cytometer (Beckman Coulter). For analysis, CD11b+ cells were gated and the percentage of GFP+ cells determined. For immunofluorescence, macrophages were plated on fibronectin (MilliporeSigma) coated glass slides (VWR). After 45 minutes, fibroblasts were added and incubated with macrophages for one hour. After that, slides were vigorously washed three times with cold PBS, followed by a regular stain of cells plated on glass slides as described elsewhere in the method section.

Measuring phagocytosis in vivo. Cancer cells were subcutaneously injected, and tumors were harvested after 14 days. Upon euthanasia, the back skin was carefully detached from the underlying tissue and the tumors were visualized. Tumors were separated from the subcutaneous skin, minced with scissors and digested in 500 μl of DMEM supplemented with 40 μl of a 12.5 mg/ml liberase (Roche) solution for 30 minutes on a shaker at 37° C. The digestion reaction was quenched with DMEM/HPL medium, and the digestion mix and tissue pieces were filtered through a 70 μm filter (Falcon). Upon washing and spinning down the samples two times, cell suspensions were incubated with primary antibodies against CD11b for 45 minutes on ice. Cell suspensions were washed with FACS buffer, resuspended in FACS buffer and analyzed either on a BD Aria Sorter or a Cytoflex (Beckman Coultier). The analysis was performed with FlowJo (FlowJo Inc). To measure a phagocytosis score, the percentages of GFP+ macrophages and phagocytosed GFP+ cells were combined. The highest individual phagocytosis score per cell line represented the value 1 and the other phagocytosis scores were normalized to this value.

MACS. To sort cells via MACS, cells were trypsinized, washed and stained with a biotin-conjugated CD63 antibody for 45 minutes in facs buffer. Cells were washed with FACS buffer, and incubated with anti-biotin beads (Miltenyi Biotec). Upon washing, cells were transferred into MS columns (Miltenyi Biotec). The flow through was then transferred into another MS column. The flow through was washed with FACS buffer, centrifuged at 300 G at 4° C. The supernatant was aspirated and cells seeded into cell culture dishes.

Flow cytometry. For live cells, single cell suspensions were washed with FACS buffer (PBS+2% FBS+1% Penicillin/Streptomycin+1 mM EDTA+25 mM HEPES) and then stained the cells with the primary antibodies for 45 minutes. Cells were washed and subsequently resuspended with FACS buffer. Either the CytoFlex Flow Cytometer (Beckman Coulter) for analysis only, or the BD FACS Aria Ill (Becton, Dickinson and Company was used. The data analysis was preformed with FlowJo (FlowJo, LLC).

ELISA of CD63 in cell supernatant. To measure CD63 in the supernatant of lung cancer cells and lung fibroblasts, a Human CD63 ELISA Kit (Thermo Scientific) was used and performed according to the manufacturer's specifications. 20,000 cells were plated into the wells of a 24-well-plate in 200 μl of serum-free DMEM/F12 supplemented with PVA and Penicillin-Streptomycin. After 24 hours, the supernatant was harvested and frozen until the supernatant from all desired cultures was collected. Before running the assay, the supernatants were diluted with 100 μl of Diluent B and centrifuged at 14,000 g for 2 minutes. All steps and incubation periods were performed according to the specifications in the manual, including an initial 2.5 incubation in the supplied plate, followed by 1 hour incubation in the biotinylated antibody reagent, 45 minutes incubation in the Streptavidin-HRP solution and 30 minutes in the TMS substrate. Between each incubation step, the plate was washed four times with 300 μl of wash buffer. After incubating the plate with the TMS substrate, the reaction was stopped with the Stop solution and absorbances were measured with an Infinite plate reader (Tecan).

EdU Assay. EdU was measured with the Click-it EdU AF594 (or AF647) Kit (invitrogen). 10,000 cells were plated in the wells of a 24-well-plate in 500 μl of DMEM/HPL medium. After 24 hours, EdU (Invitrogen) was added directly to the wells. Cells were harvested after 45 and 90 minutes and fixed in 4% PFA (Electron Microscopy Service) for 15 minutes at room temperature. Cells were washed with PBS and incubated for 15 minutes in saponin-based wash and permeabilization buffer, followed by addition of the EdU reaction mix, another washing step with the wash and permeabilization buffer, and resuspension in wash and permeabilization buffer. Samples were analyzed on a Cytoflex (Beckman Coultier).

Transfection with sgRNA/Cas9 mRNA. Modified sgRNA against CD63 was purchased from Synthego after using their knockout designer. The sequence of sgRNAs are presented in Table 1. Upon receival of the sgRNA, they were diluted in nuclease-free H2O. A RNP transfection was preformed with the transit-mRNA transfection kit (Mirus) and Alt-R S.p. HiFi Cas9 Nuclease V3 (Integrated DNA Technologies). One day before the transfection, 25,000 target cells were plated into the wells of a 24-well plate in antibiotic-free DMEM medium with 10% FCS. After warming up Optimem medium to room temperature (Gibco), 7.5 μg of sgRNA and 0.56 μl of Cas9 were added to 64 μl of Optimem, followed by mixing through pipetting up and down and an incubation of 30 minutes. Before reaching the end of the 30 minutes, 50 μl of DMEM were pipetted into 1.5 ml tubes, followed by adding first 1.5 μl of transit-mRNA (Mirus) and second 1.5 μl of mRNA Boost (Mirus). This mixture was vortexed, and 15 μl of the RNP mix were added to each tube. After shortly vortexing these mixtures, they were incubated for 2 minutes. Then, they were added drop-wisely onto the target cells, and thoroughly mixed with the medium through pipetting up and down. The cells were incubated for 2 days, before transferring the cells into the wells of 6-well plate or a 10 cm dish. For the purification of knockout cultures, either FACS for cancer cell cultures or MACS for fibroblasts was used.

TABLE 1
sgRNA sequences

| [symbol] | | | | | | |
|---|---|---|---|---|---|---|
| EQ | | [symbol] | | | | |
| ID | | ene | | [symbol] | | |
| NO | Design ID | Symbol | Position | trand | Sequence | AM |
| | Hs.Cas9.C | [symbol] | 55726719 | | CAACCACACTGCTTCGATCC | GG |
| | D63.1.AA | D63 | | | | |
| | Hs.Cas9.C | [symbol] | 55727223 | | CCCACTGCGATGATGACCAC | GG |
| | D63.1.AB | D63 | | | | |

[symbol]

indicates data missing or illegible when filed

Plasmid transfection. The CD63-eGFP plasmid was a gift from Paul Luzio and was purchased from addgene. Upon arrival, the bacteria stab was plated on kanamycin (MilliporeSigma) containing agar plates, followed by transferring single colonies into 14 ml tubes with LB medium with kanamycin. The next day, the bacteria-containing medium was transferred into flasks with 200 ml of LB medium with kanamycin. The next day, a maxiprep (Qiagen) was preformed according to the manufacturer's specifications. The final plasmid was diluted in TE buffer. For transfecting lung fibroblasts, 50,000 fibroblasts were plated into the wells of a 6-well-plate. The next day, 6 μg of plasmid DNA was mixed with 18 μl of Fugene (Promega) in 300 μl of DMEM medium. After an incubation of 15 minutes, the mixture was added drop-wisely directly onto the cells. The cells were incubated with the plasmid for two days before proceeding to the next experiments.

Tissue fixation and hematoxylin staining. The harvested tissue was kapt in 10% formalin overnight. Tissue was then submitted to the Stanford Human Pathology/Histology Service Center for paraffin-embedding and cutting. The tissue slides were deparaffinized and rehydrated with xylene and a descending ethanol row. After washing the slides in PBS, they were incubated in hematoxylin (American MasterTech) for 4 minutes, then in bluing reagent (ThermoFisher Scientific) for 2 minutes and in Harleco® (MilliporeSigma) for 2 minutes. Slides were dehydrated with ethanol and xylene (MilliporeSigma) and mounted wit Permount® (ThermoFisher Scientific).

Immunofluorescence staining of paraffin-embedded sections. When using paraffin-embedded tissue, the tissue was first deparaffinized and rehydrated. Then, antigen retrieval was preformed with a citric acid buffer in a pressure cooker for 30 minutes, followed by blocking with 5% serum. Sections were incubated with the primary antibody over night at 4° C. After washing in PBST, the sections were incubated with the secondary antibody at room temperature for 30 minutes under agitation. Sections were washed, counterstained with DAPI and mounted with fluoromount (SouthernBiotech). Images of histological slides were obtained on a Leica Eclipse E400 microscope (Leica) equipped with a SPOT RT color digital camera model 2.1.1 (Diagnostic Instruments).

Immunofluorescence staining of cells plated on glass slides. For plating cells on glass slides (VWR), areas were encircled on glass slides with a fat pen (Vector Laboratories), followed by sterilization for at least one hour under UV light. Then, fibronectin (20 μg/ml PBS) was added. After incubating the slides for one hour in a cell incubator at 37° C., the fibronectin solution was aspirated, and the slides were dried in the cell culture cabinet. Cells were added onto the glass slides and incubated for different amount of times in regular cell culture medium. Thereafter, the glass slides were washed in PBS, followed by fixing the cells with 4% PFA at room temperature for ten minutes. The slides were washed again in PBS, followed by permeabilization in TritonX for ten minutes for intranuclear stainings. Otherwise, cells were blocked with 5% serum for one hour. Afterwards, the staining procedure equaled the staining protocol of the paraffin-embedded slides.

Immunohistochemistry. When using paraffin-embedded tissue, the tissue was first deparaffinized and rehydrated. Then, antigen retrieval was preformed with a citric acid buffer in a pressure cooker for 30 minutes, followed by blocking with 5% serum. Sections were incubated with the primary antibody over night at 4° C. After washing in PBST, the sections were incubated with the secondary HRP-antibody at room temperature for 30 minutes under agitation. Sections were washed in PBST and ddH2O followed by an incubation up 20 minutes in AEC Peroxidase Substrate (Abcam). Sections were washed in ddH2O and incubated in Modified Mayer's Hematoxylin for 4 Minutes. After washing in ddH2O, slides were incubated for 4 minutes in 0.02% Ammonium hydroxide, washed in ddH2O, and mounted with fluoromount (SouthernBiotech).

In Situ Hybridization. In situ hybridization was performed with the RNAScope® 2.5 HD Assay (Advanced Cell Diagnostics) according to the manufacturer's specifications. Tissue sections were deparaffinized in xylene for 15 minutes, followed by 100% ethanol for 1 minute. After drying, the sections were incubated with hydroxide peroxidase for 10 minutes. After washing in deionized water, the sections were cooked for 15 minutes in the antigen retrieval buffer in a pressure cooker. The slides were then washed in deionized water and incubated in 100% ethanol for 1 minute. The next day, sections were incubated with Protease Plus for 30 minutes. After being washed in deionized water, the sections were incubated with the probes against CD63 (C1) and FSP1 (C2) for 2 hours at 37° C. in a humidified chamber, followed by incubations with the amplification buffers 1, 2, 3, 4, 5 and 6, the Red staining solution, amplification buffers 7, 8, 9, and 10, and the Green staining solution. Upon washing in the RNAScope Wash buffer, the cells were counterstained with Gill No. 1 hematoxylin solution (diluted 1:1 with deionized water) (MilliporeSigma) for 2 minutes, followed by 2 minute incubation in 0.02% Ammonium hydroxide in deionized water. The sections were then dried in an oven at 60° C., dipped into xylene, and mounted with VectaMount Permanent (Vector Laboratories).

Proximity-Ligation Assay. To determine physical proximity, a Duolink™ In Situ Red Starter Kit (MilliporeSigma) was used. Target cells were plated and macrophages on fibronectin-coated glass slides for one hour, before carefully washing the slide in PBS, followed by an incubation for 10 minutes in 4% PFA, another washing step in PBS, and 60 minutes incubation in PBS with 5% donkey serum. Then, the primary antibodies against CD63 (Abcam, clone TS63) and CD9 were resuspended in PBS with 1% donkey serum and incubated over night at 4° C. Single stain controls were used to determine specificity. The rest of the procedure was performed as outlined in the manufacturer's specifications. The next day, slides were washed with wash buffer, incubated for 1 hour with the PLA probes at 37° C., washed wish wash buffer, incubated for 30 minutes with the ligase at 37° C., washed with wash buffer A, incubated with the polymerase (amplification step) for 100 minutes at 370 C, followed by two washing steps in wash buffer B. Samples were mounted with Duolink In Situ Mounting Medium, covered with a cover glass and imaged with a confocal microscope.

Preparation of tissue microarrays. To prepare the tissue microarray, the Stanford Pathology database was searched for resected lung adenocarcinomas. Blocks and slides with H&E stains were requested and suitable areas of interest identified. For the TMA, cores were transferred from the original block into a paraffin block. The paraffin block was melted at 55° C. for one hours, before being cut.

Western Blotting. For Western Blotting, 100000 cells were plated in 6-well-plates. 24 hours thereafter, cells were washed with PBS and lysed in 200 μl of cell lysis buffer (CST). The lysate was then sonicated and centrifuged at 18,000 g for 10 minutes at 4° C. The supernatant was transferred into new tubes. Protein concentrations were calculated with the BCA Protein Assay Kit (Pierce). Then, 10 μg of protein were diluted with 10 μl of LDS Sample Buffer (Millipore Sigma), 4 μl of Bolt Sample Reducing Agent (ThermoFisher), and ddH2O up to a volume of 40 μl. The samples were heated to 95° C. for 7 minutes and cooled down on ice. A mini gel tank system (Invitrogen) was used for running the gel and blotting the protein. The samples were transferred into the wells of Bolt 12% Bis-tris Plus gels (Invitrogen). The gels were then run at 160 V for 65 minutes in Bolt Running Buffer (ThermoFisher). Afterwards, the gel was transferred onto nitrocellulose (mdl Membrane Technologies). Blotting was performed in Mini Blot Modules (Invitrogen) at 350 A for 60 minutes at RT with Efficient Western Transfer Buffer (G Biosciences). The membranes were then washed in PBS, blocked with 5% skim milk, and incubated with the primary antibodies overnight at 4° C. on a rocking platform. The next day, the membranes were washed in PBST, incubated with the secondary antibody for 30 minutes at RT on a rocking platform, and washed again with PBST. Finally, the blots were incubated with Luminata Forte Western HRP Substrate for 5 minutes (MilliporeSigma) and developed on X-ray films (Advansta). If the membranes were stained with another round of primary antibodies, the membranes were stripped with Western Blot Strip-II Buffer (Advansta) and washed with PBST.

Primary antibodies. Flow cytometry: CD11b (Biolegend, #301305, Clone ICRF44), CD31 (Biolegend, #102522, Clone MEC13.3), CD45 (Biolegend, #103109, Clone 30-F11), CD63 (Biolegend, #143906, Clone NVG-2), CD63 (Biolegend, #353007, Clone H5C6), CD86 (Biolegend, #374205, Clone BU63), CD163 (Biolegend, #333619, Clone GHI/61), CD204 (Biolegend, #371907, Clone 7C9C20), CD206 (Biolegend, #321103, Clone 15-2), CD326 (Biolegend, #118217, Clone G8.8)

MACS Sorting: CD63 (Biolegend, #353017, Clone H5C6). Immunostainings: CD9 (Abcam, #ab92726), CD63 (Abcam, #ab59479, Clone TS63), CD63 (Abcam, #ab1318, Clone NK1/C3), CD63 (R&D, #MAB5417), CD81 (Abcam, #ab128202), CD151 (Abcam, #ab33315, Clone 11G5a), FSP1 (abcam, #ab58597), FSP1 (MilliporeSigma, #07-2274), GFP (Abcam, #ab183734), Ki67 (Abcam, #ab15580)

Secondary antibodies. Immunostainings: AF488 donkey anti-mouse (Invitrogen, #A-21202), AF594 donkey anti-goat (Novus, #NBP1-75607), AF594 goat anti-rat (Invitrogen, #A-11007), HRP goat anti-rabbit (Abcam, #ab205718), HRP donkey anti-mouse (Abcam, #ab6820).

Western Blot: CD9 (Abcam, #ab92726), CD63 (Abcam, #ab59479, clone TS63), CD81 (Abcam, #ab128202), CD151 (Abcam, #ab33315, Clone 11G5a), CTNNB1 (CST, clone D10A8), GAPDH (GeneTex, clone GT239), JNK2 (CST, clone 56G8), MCM2 (CST, #4007), Notch1 (CST, clone D1E11), PI4K (Santa Cruz Biotechnology, clone B-5), Syncytin1 (Invitrogen, BS-2962R)

Calculating Kaplan Meier Plots. To calculate Kaplan Meier Plots, the Lung Cancer mRNA data base on the Kaplan-Meier Plotter and screened for CD14, CD63, CD64, and CD68. Patients with adenocarcinomas were split into two groups based on the median expression of the specific gene. Correlations were calculated with the Spearman correlation.

Statistics. Graphpad prism (Graphpad Software Inc) was used for creating graphs and running statistical analyses. When two values were compared, a two-sided student's t-test was used, if more than two values were analyzed, ANOVA tests were used with corrections for multiple comparisons. Experiments included at least three independent values. Hypotheses were tested through at least two independent experiments. Experiments with different cell lines were regarded as independent experiments. P values below 0.05 were considered as statistically significant.

### Example 3

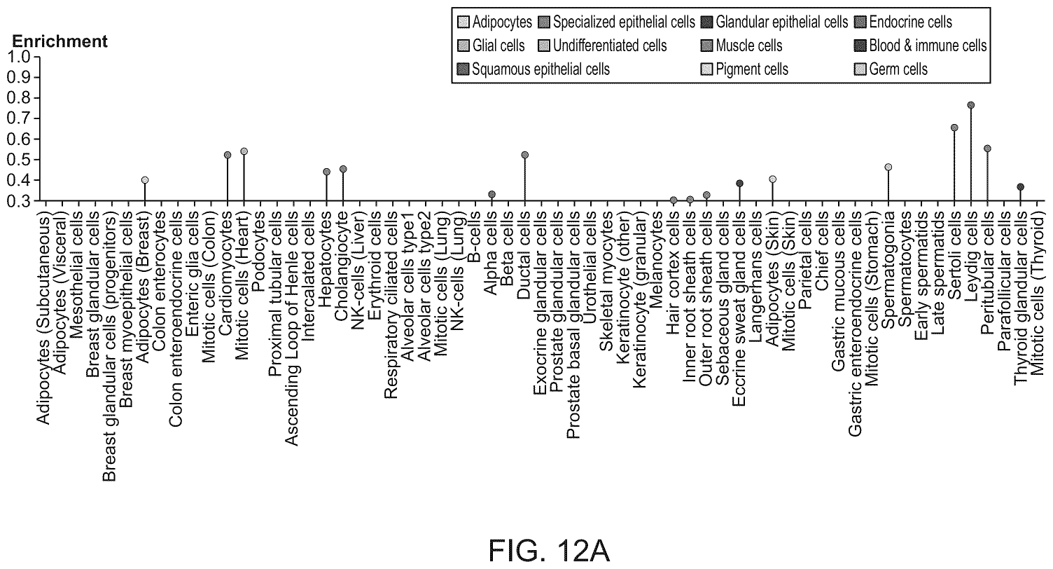

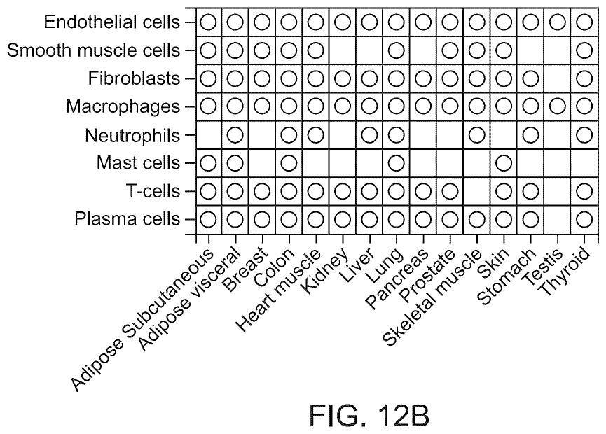

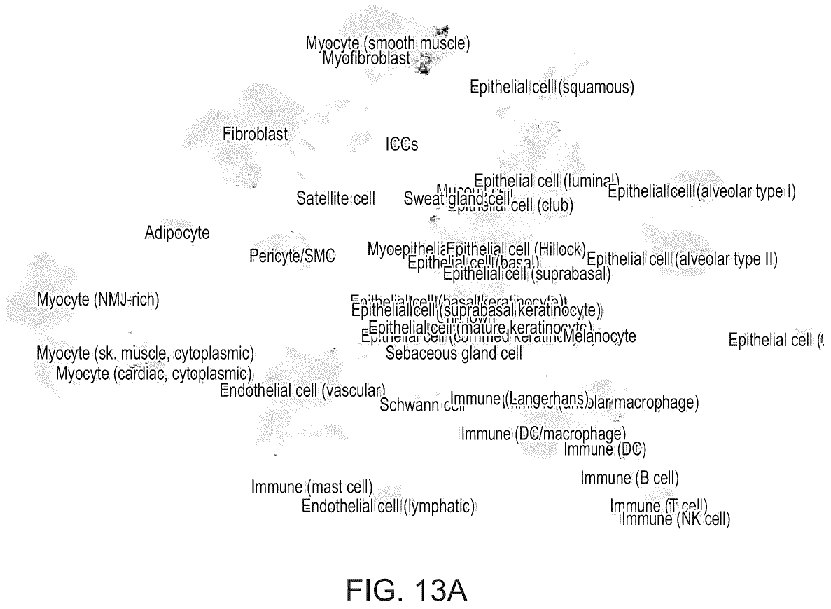

CD63 is expressed in a number of healthy tissues and cells types. As disclosed herein, the specific tissues and cell types are shown in FIG. 12.

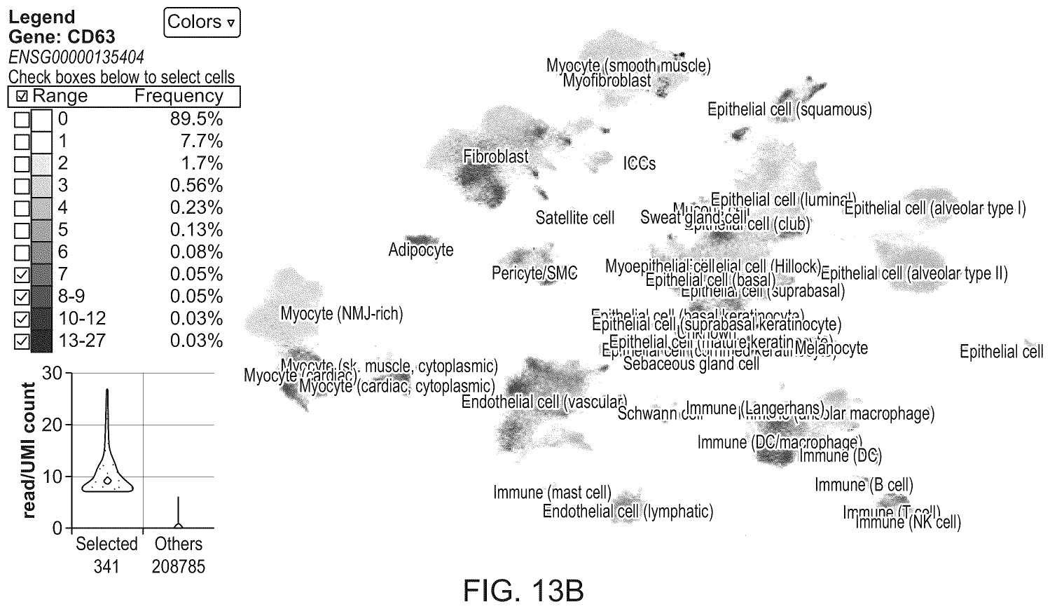

As shown in FIG. 13, a cross-tissue atlas of 209,126 nuclei profiles were generated using four snRNA-seq methods that were applied to 25 samples from 16 donors and a map the expression of CD63 in human healthy tissues was labeled by dark dots. These data confirm that CD63 expression is rare in healthy human tissues and mainly present in a minor subset of myofibroblasts, myocytes and epithelial cells.

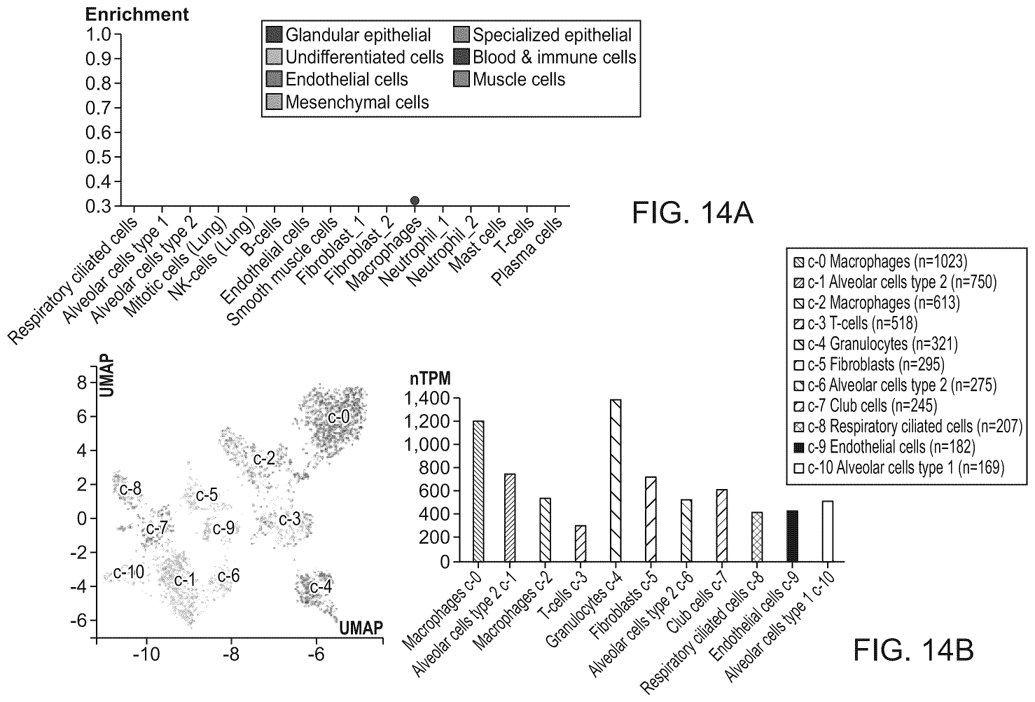

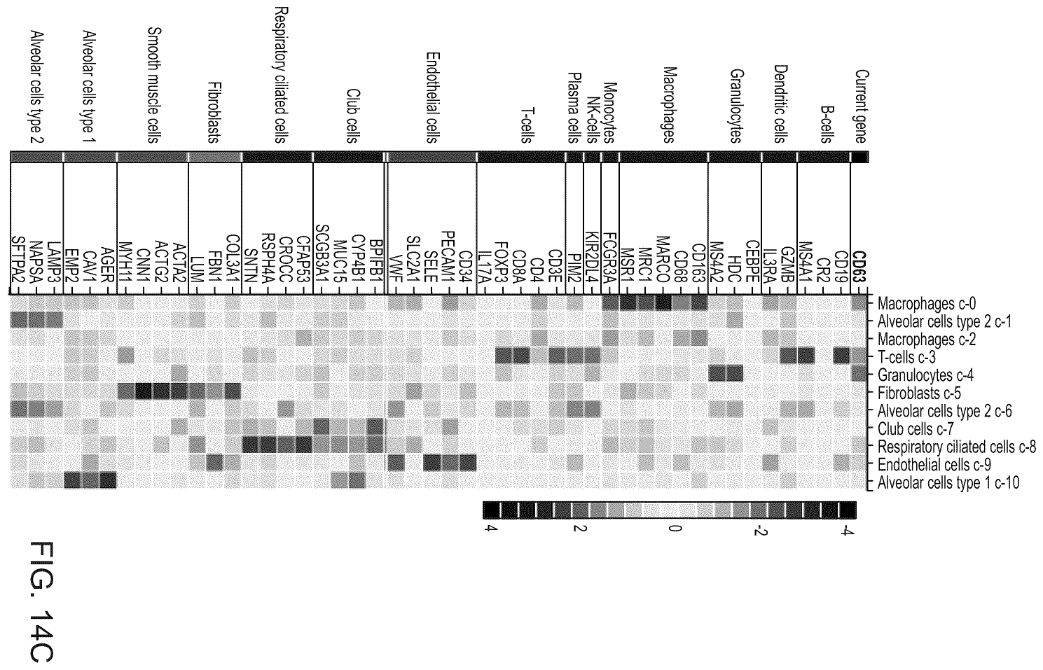

A Meta-analysis of scRNA seq data of healthy lung tissue (GSE130148) was preformed and revealed no significant expression of CD63 in healthy lung except low expression in macrophages (FIG. 14 A). A heatmap was generated and gene expression analysis was preformed which depicted single cell data according to cell type as scatter plot, all cells are shown in the color scale as % of maximal expression (FIG. 14 B-C).

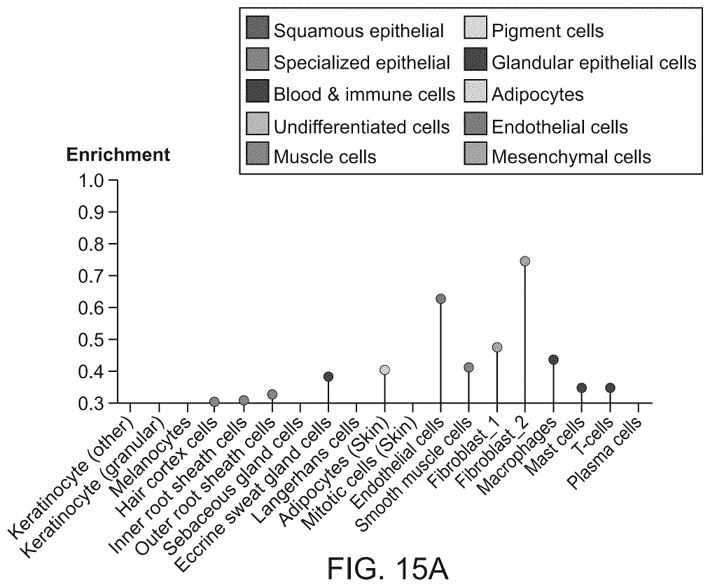

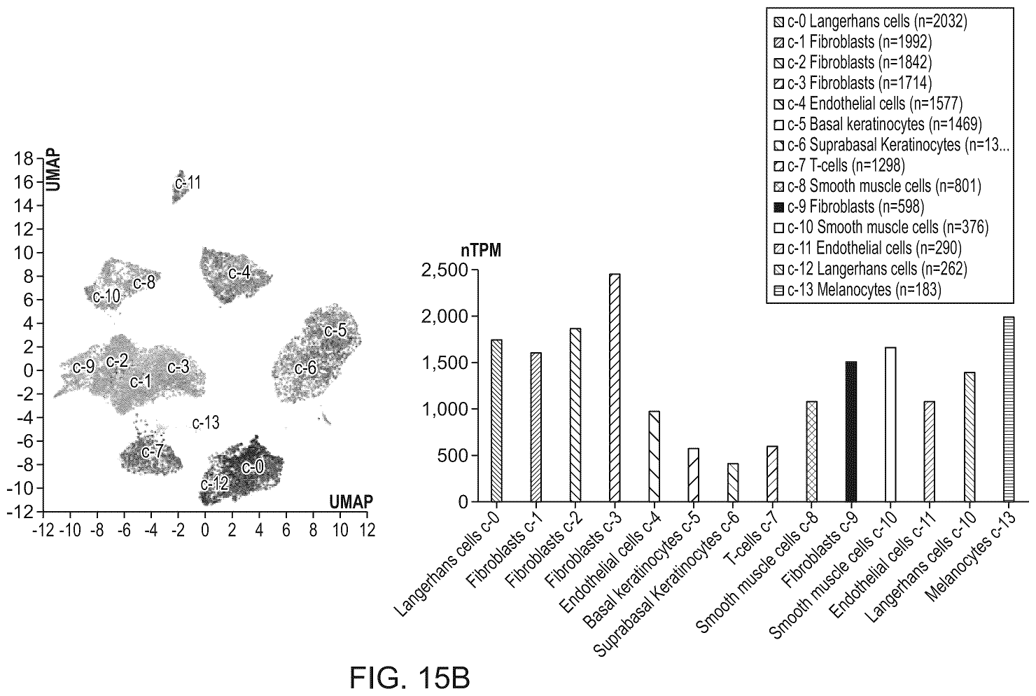

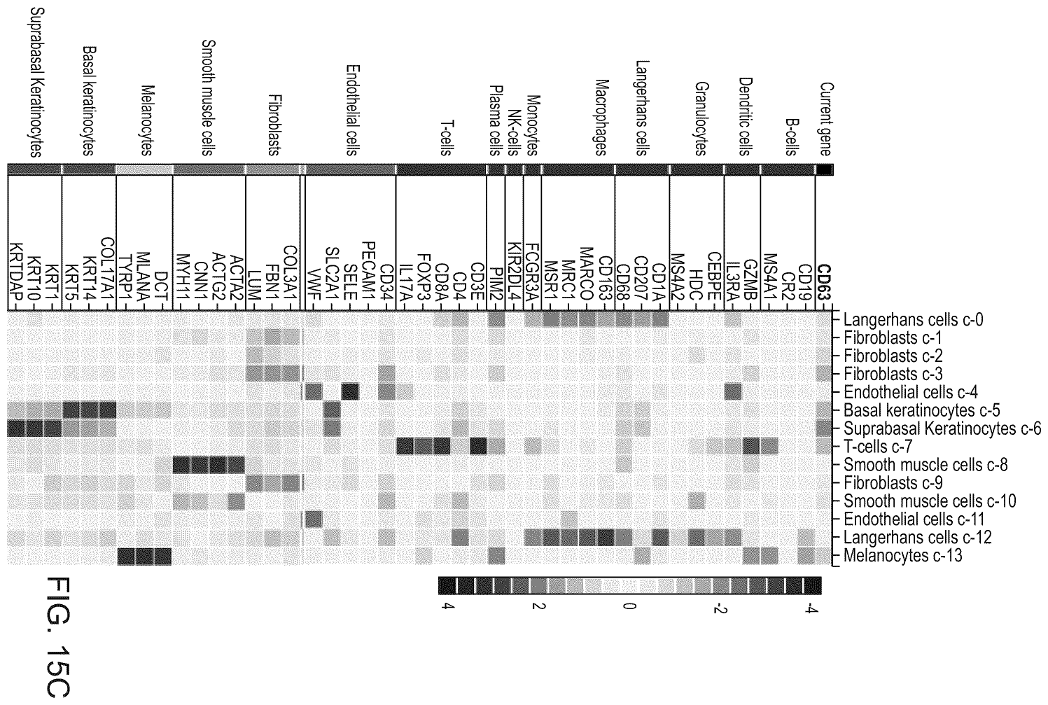

As shown in FIG. 15, a Meta-analysis of scRNA seq of human healthy skin demonstrates CD63 expression to be high in fibroblasts followed by endothelial cells. (FIG. 15 B) UMAP and (FIG. 15 C) heatmap gene expression analysis of GSE130973 demonstrate scatter plots colored according to cell type group, in addition all cells are shown on the color scale as % of maximal expression.

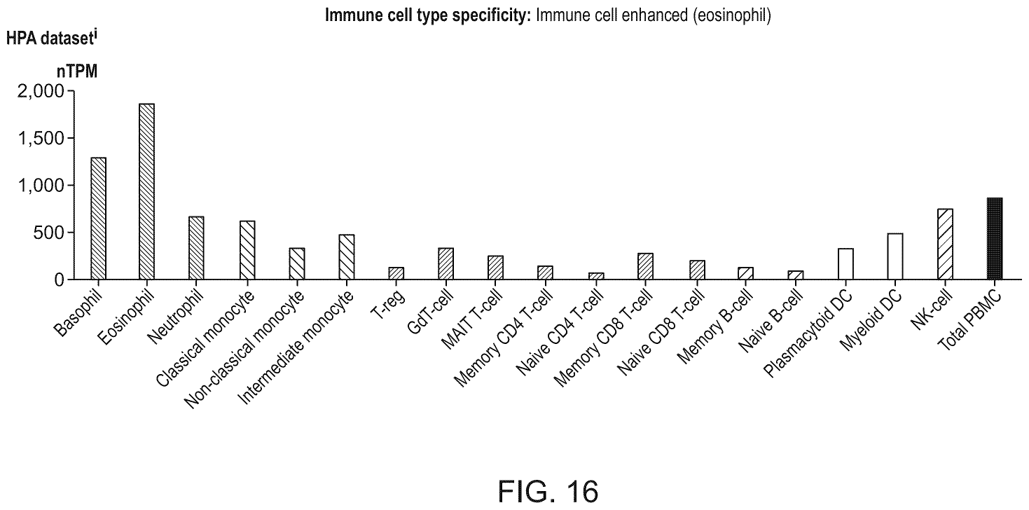

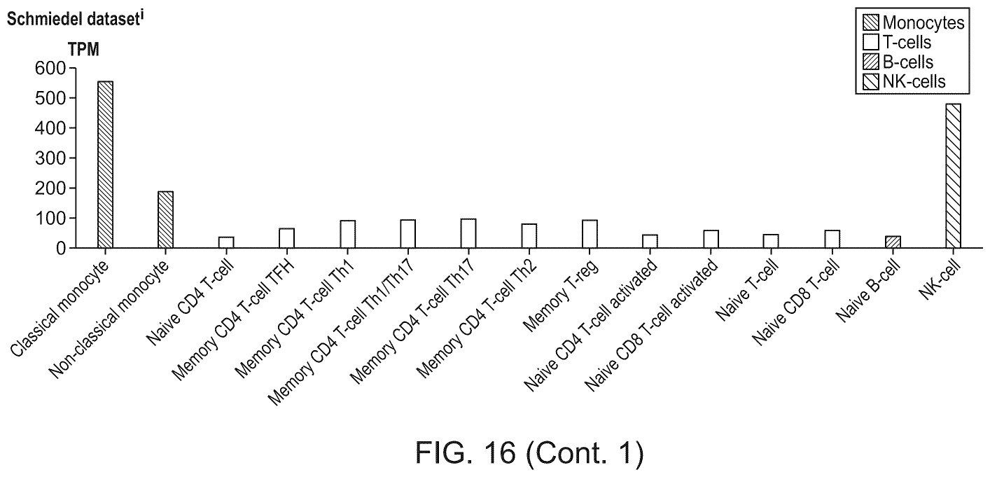

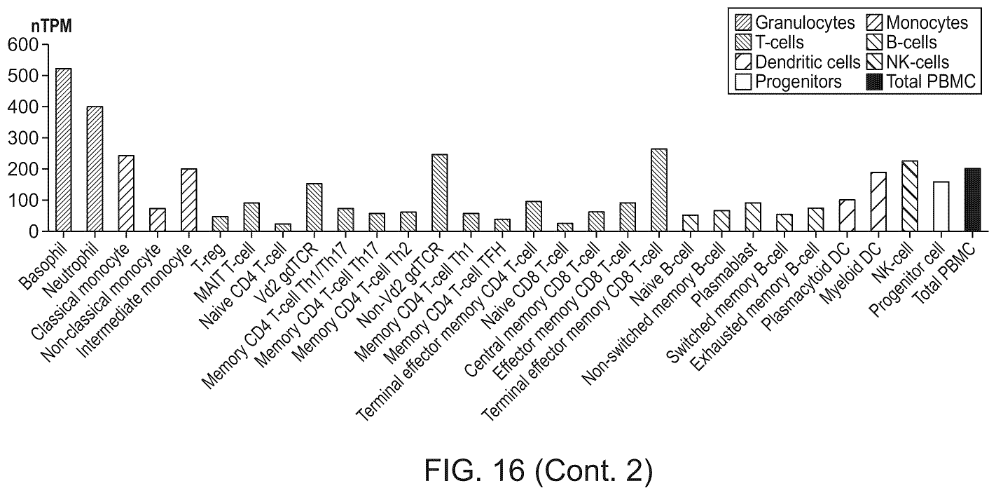

The estimate of the transcript abundance of each protein-coding gene disclosed in FIG. 16 was determined by RNA-seq analysis using three data sets, 69 cell lines, 52 human tissues and 18 blood cell types and peripheral blood mononuclear cells (PBMC). The nTPM number quantifies CD63 expression in immune cells across these data sets.

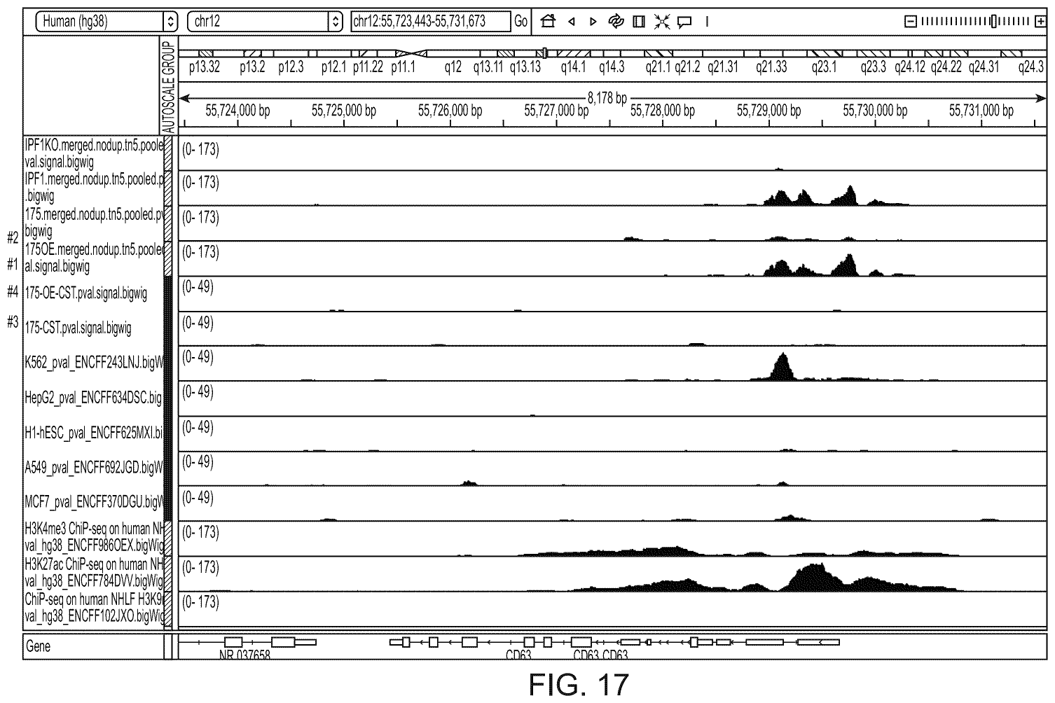

Increased chromatin accessibility of the promoter regions of CD63 in lung fibroblasts derived from lung fibrosis patients was observed following ATAC and ChIP-seq as shown in #1 and 3 in FIG. 17. When JUN was knocked down with CRISPR-Cas9, chromatin accessibility of the CD63 promoter was reduced (#2 and 4 in FIG. 17) suggesting that JUN regulates promoter accessibility of CD63 in lung fibroblasts in lung fibrosis.

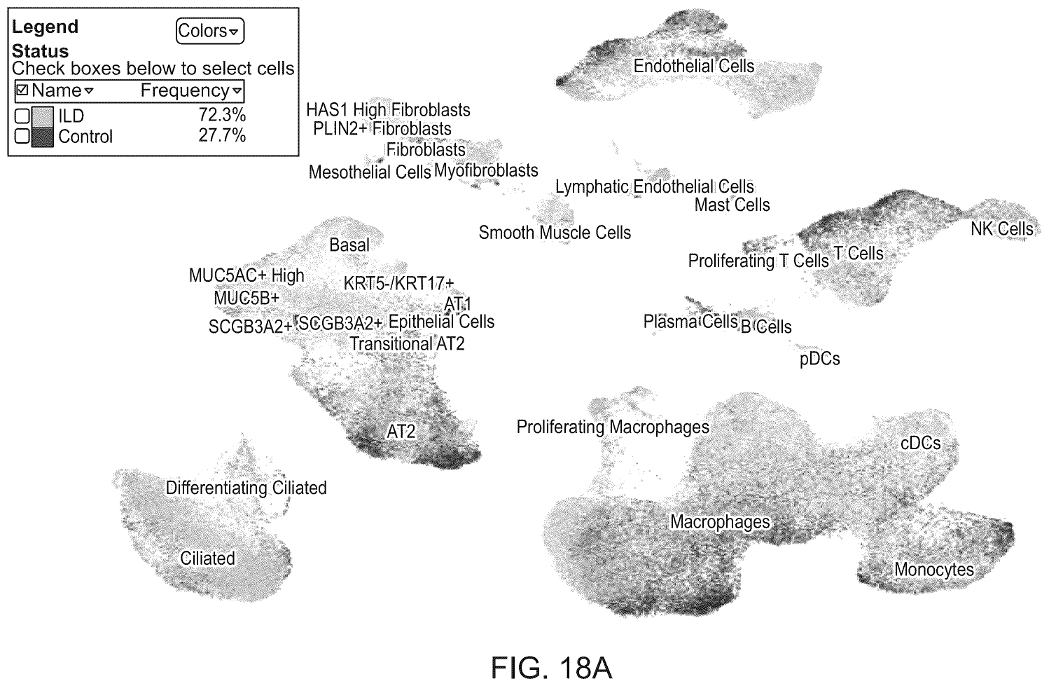

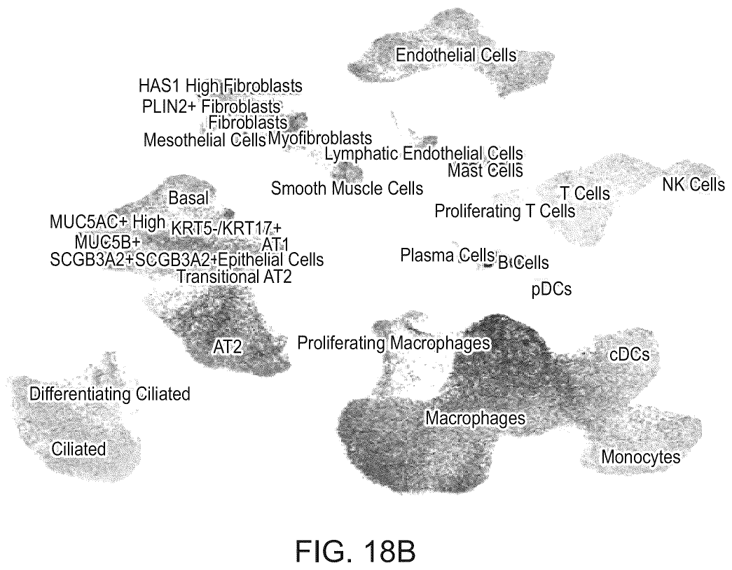

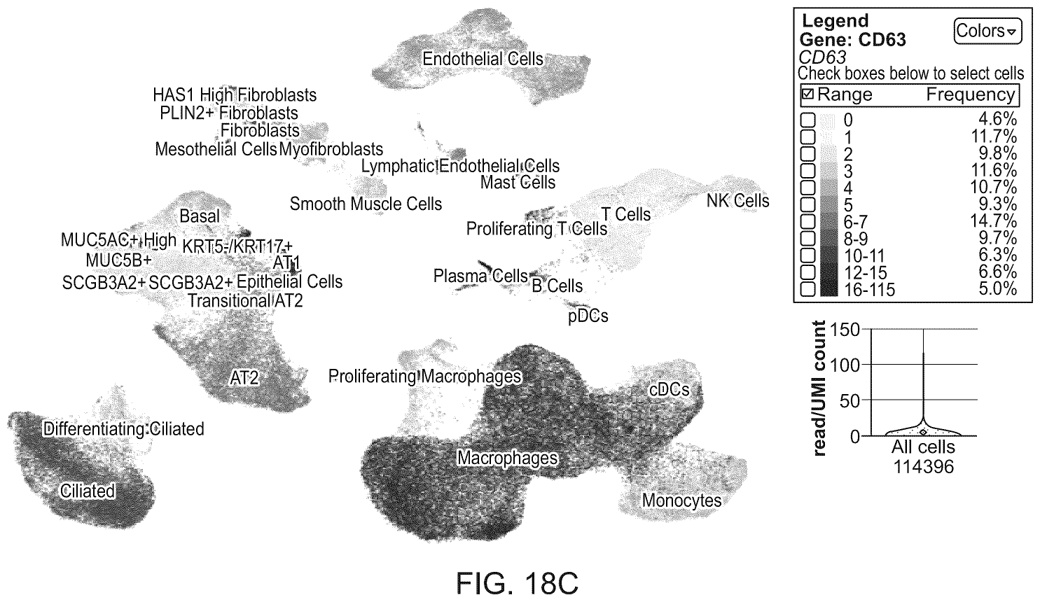

As shown in FIG. 18, 31 distinct cell types and CD63 expression in macrophages and epithelial cells were identified through analysis of 114,000 cells from 20 fibrotic lungs and 10 control lungs GSE135893. FIG. 18 A-B disclose cell distribution in interstitial lung disease/lung fibrosis (ILD) and non-fibrosis healthy lung (Control). FIG. 18 C discloses CD63 expression in the lung is quantified as % maximum expression across all cell types.

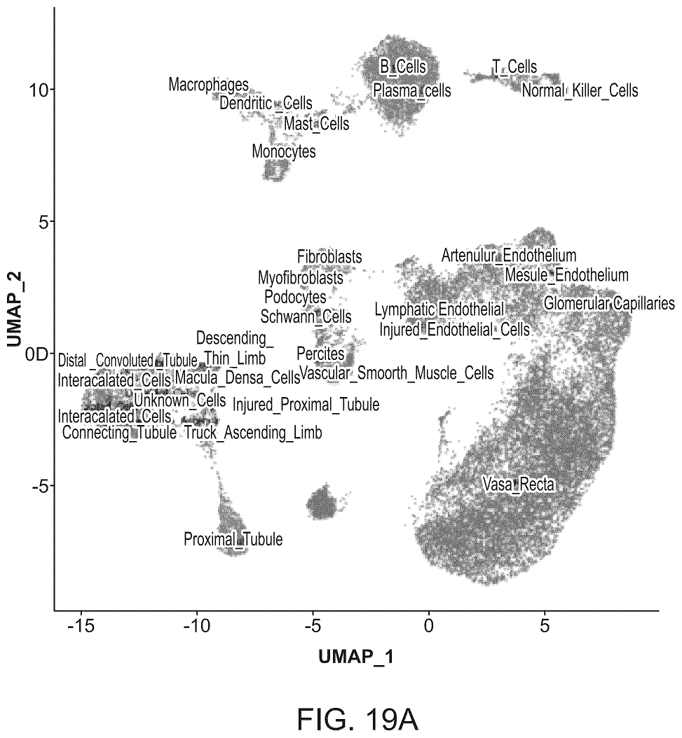

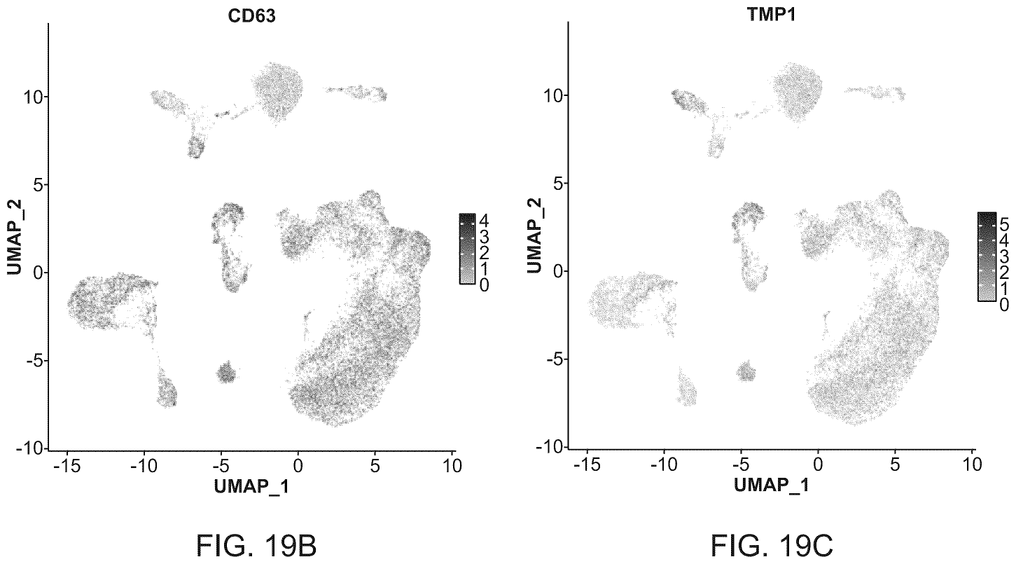

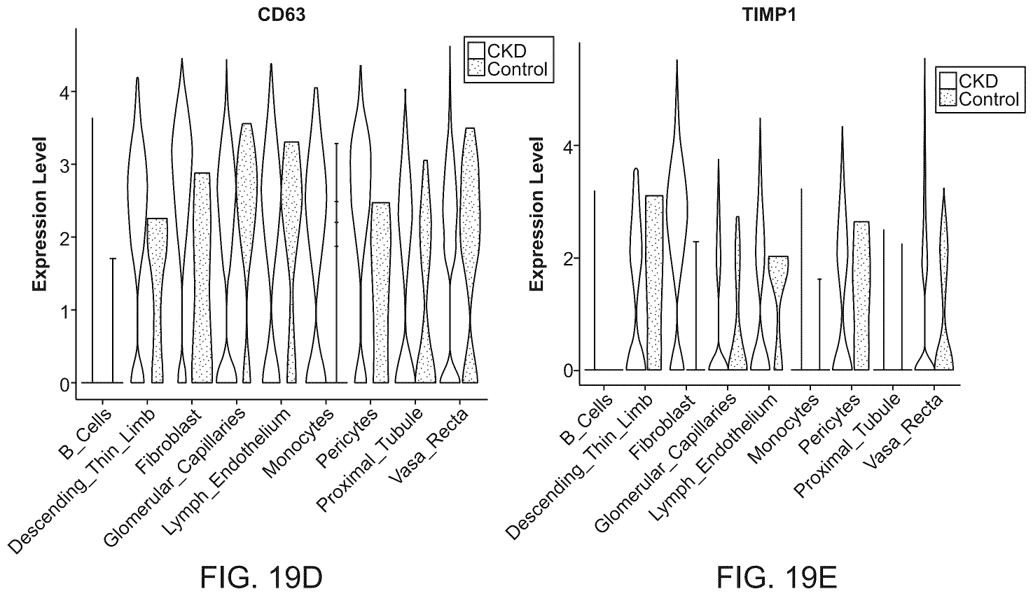

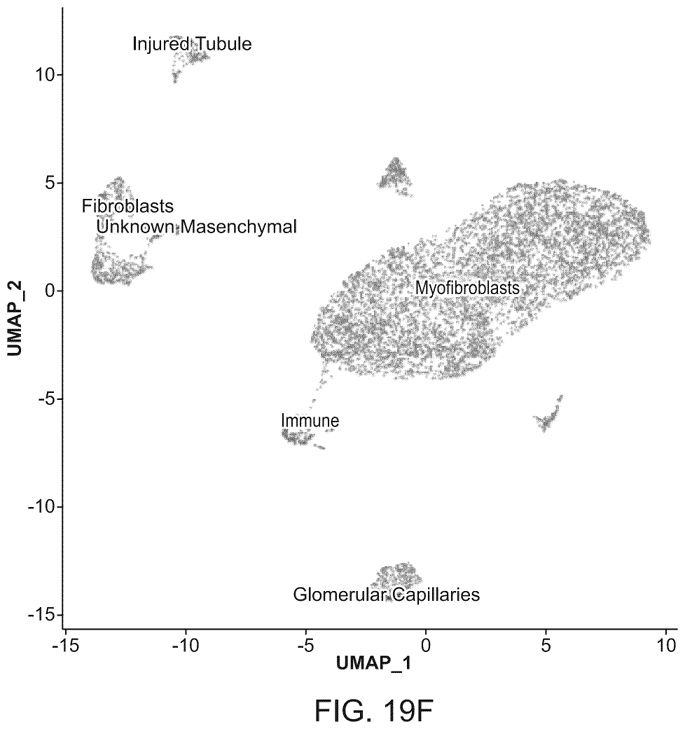

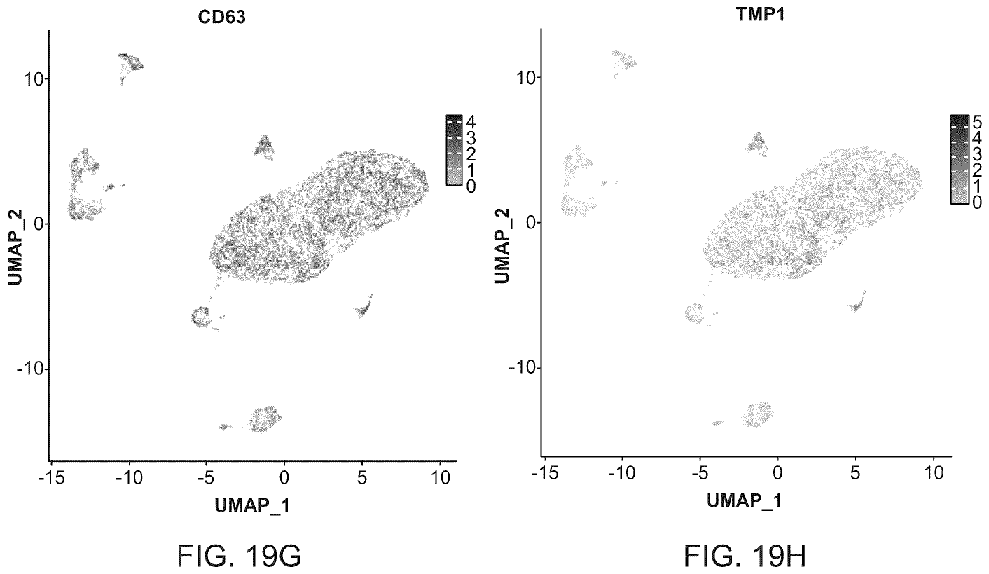

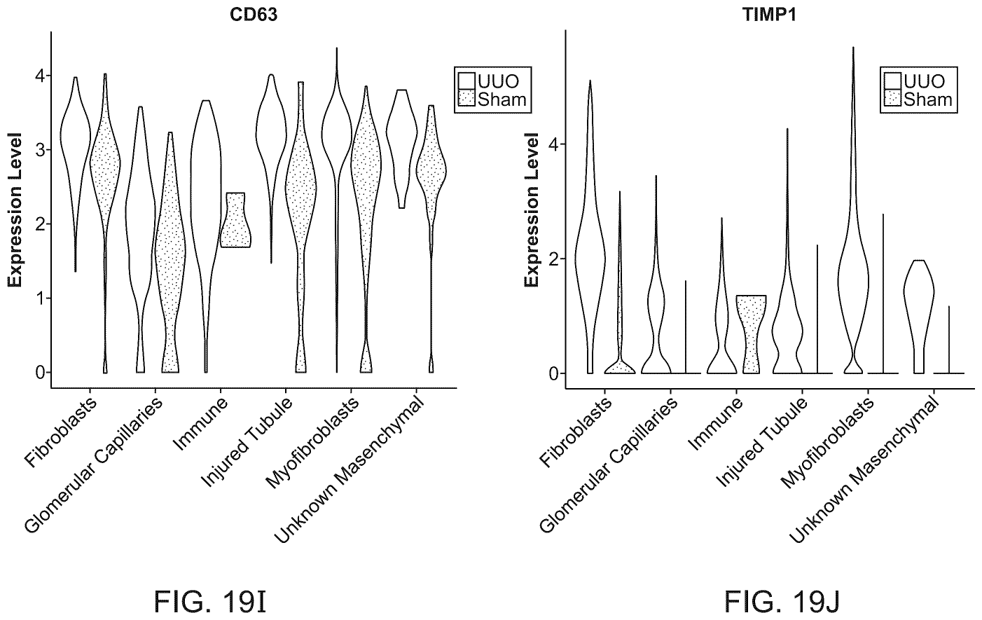

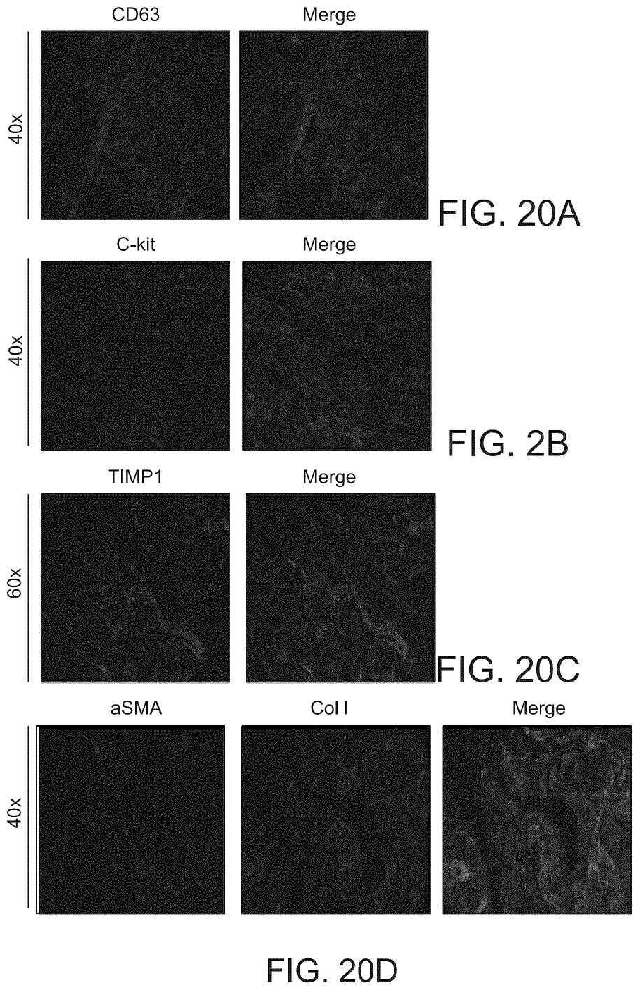

FIG. 19 discloses a single-cell atlas of human chronic kidney disease (CKD) due to hypertensive nephrosclerosis and mouse UUO-induced fibrosis model. UMAP embedding was preformed on 51,849 CD10− single cells from 15 human kidneys and it was found that CD63 is mainly expressed in monocytes, fibroblasts, proximal tubule cells and pericytes (FIG. 19 A-B). TIMP1 was found to be mainly expressed in fibroblasts and macrophages (FIG. 19 C). CD63 was found to be significantly overexpressed in monocytes, fibroblasts, descending thin limb and pericytes in CKD (FIG. 19 D). (TIMP1 was also found to be significantly overexpressed in fibroblasts in CKD (FIG. 19 E). UMAP embedding was then preformed on 7,245 PDGFRα+PDGFRβ+ cells in mouse kidney and CD63 was found to be expressed in all cell types (FIG. 19 F-G). TIMP1 was found to be mainly expressed in myofibroblasts (FIG. 19 H). (I) CD63 was found to be significantly overexpressed in mesenchymal cells and fibroblasts in UUO model (FIG. 19 I). TIMP1 was found to be significantly overexpressed in every cell type detected in UUO model (FIG. 19 J).

As shown in FIG. 20, expression of CD63 and fibrotic proteins was found in in IPF tissues. CD63 was labeled with immunofluorescence in fresh frozen lung fibrosis (IPF) tissue (FIG. 20 A). C-kit and TIMP1 was also labeled with immunofluorescence in FFPE IPF tissue (FIG. 20 B-C). C-kit and TIMP1 are known as associated proteins of CD63. αSMA and Col I were immunofluorescently labeled and found in FFPE IPF tissue (FIG. 20 D). Magnification was either 40× or 60× as labeled.

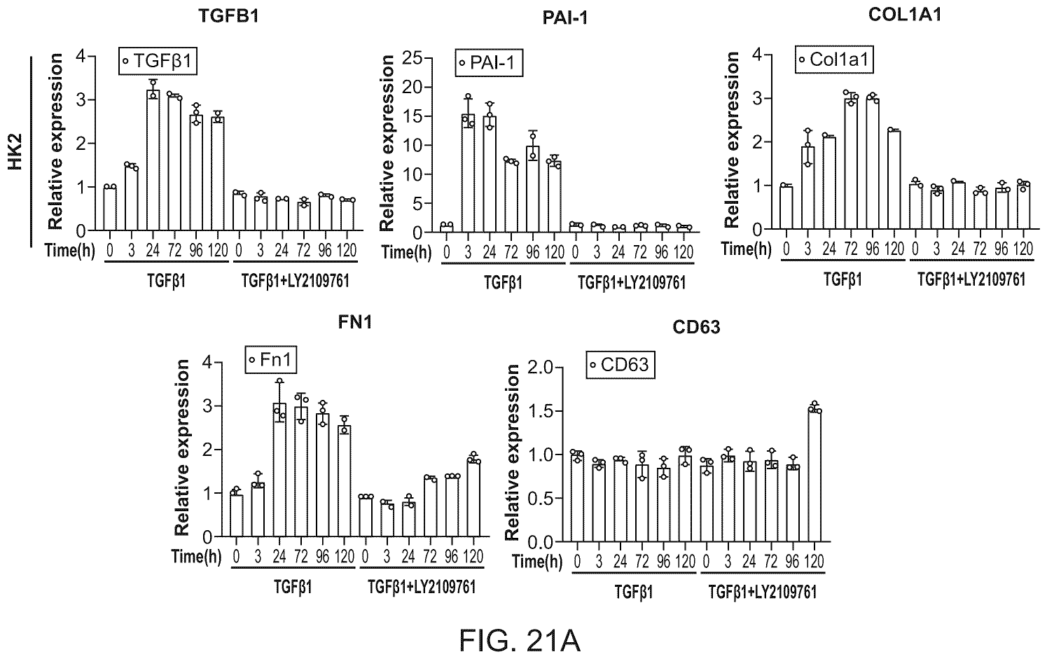

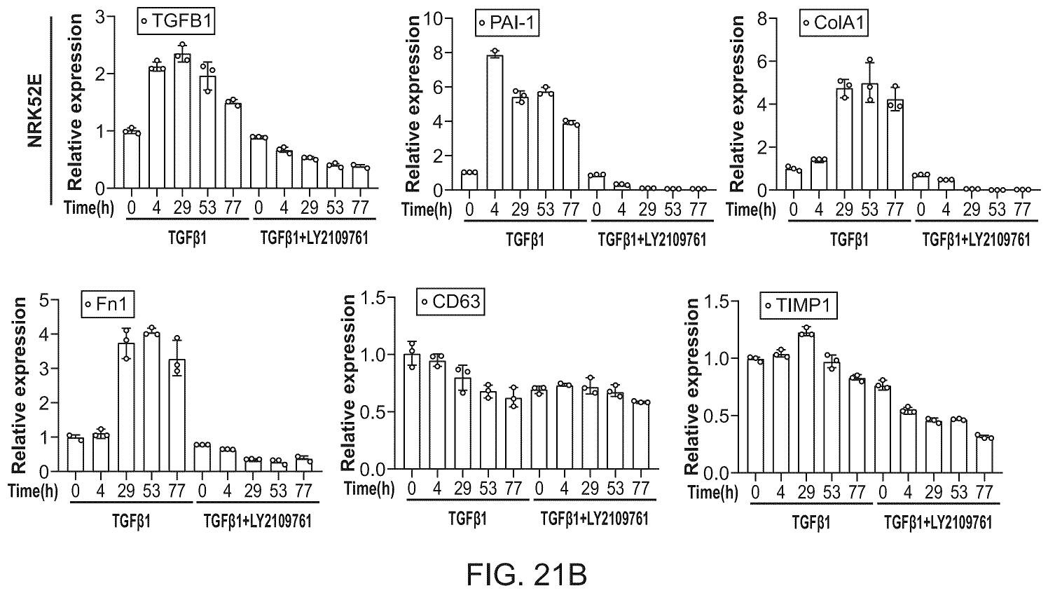

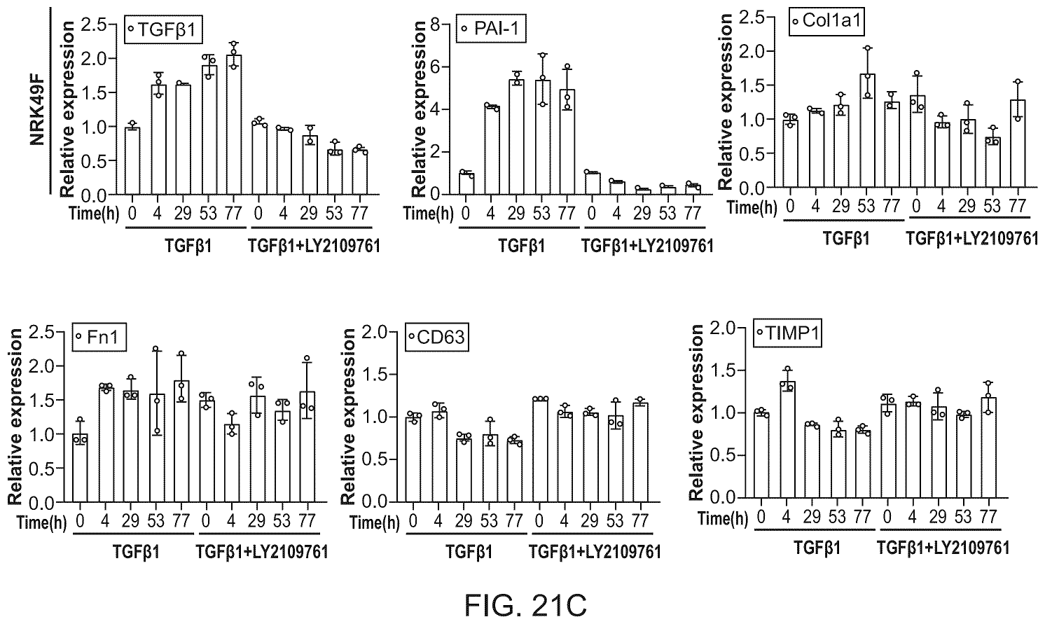

Activation of TGFβ1 signaling pathway elevated the expression of fibrotic related proteins in 3 kidney cell lines. Human epithelial (HK2), rat epithelial (NRK52E), rat fibroblast (NRK49F) cell lines were treated with TGFβ1 (10 ng/ml) or TGFβ1 (10 ng/ml) plus LY2109761 (2.5 μM) for times indicated in hours as shown in FIG. 21 A-C. RT-qPCR analysis showed the elevated expression of fibrotic related proteins. CD63 and its known interactor TIMP1 were not increased in any of the cell lines, indicating that CD63 is not involve in TGFβ1 signaling pathway.

### Example 4

Liver pathogenicity's affect the quality-of-life patients. Some of these devastating conditions include liver cirrhosis, liver cancer (hepatocellular carcinoma/HCC), non-alcoholic steatohepatitis (NASH) and non-alcoholic fatty liver disease (NAFLD) which follow liver fibrosis in the developmental process of disease progression. Hepatic fibrosis is characterized by the accumulation of proteins like collagen causing most chronic liver diseases ultimately leading to liver failure. Reversibility of advanced hepatic fibrosis has stimulated researchers to develop anti-fibrotic molecules thus preventing progression to chronic liver dysfunction. Current therapeutics involve hepatectomies and liver transplantation in combination with conventional chemotherapeutics and some targeted immune therapies like Nivolumab, Pembrolizumab and small molecule inhibitors like Sorafenib which have been approved by the FDA for therapy. However, owing to huge side-effects and lack of potency, there still exists a huge dearth of promising anti-fibrotic drugs in clinic thus warranting a need for increased development of immune therapeutic strategies for treating fibrosis especially in the context of the liver. CD63, also known as lysosome-associated membrane glycoprotein 3, is a cell surface glycoprotein and a member of the transmembrane-4 superfamily (TM4SF; tetraspan proteins). CD63 as an immune target for treating disease has not been previously described in the context of liver cirrhosis, HCC, NASH and NAFLD. Here using high throughput transcriptomics and novel in-vitro and in-vivo experimentation we show CD63 is an integral immune regulatory protein that plays a critical role in the pathogenesis of advanced liver diseases.

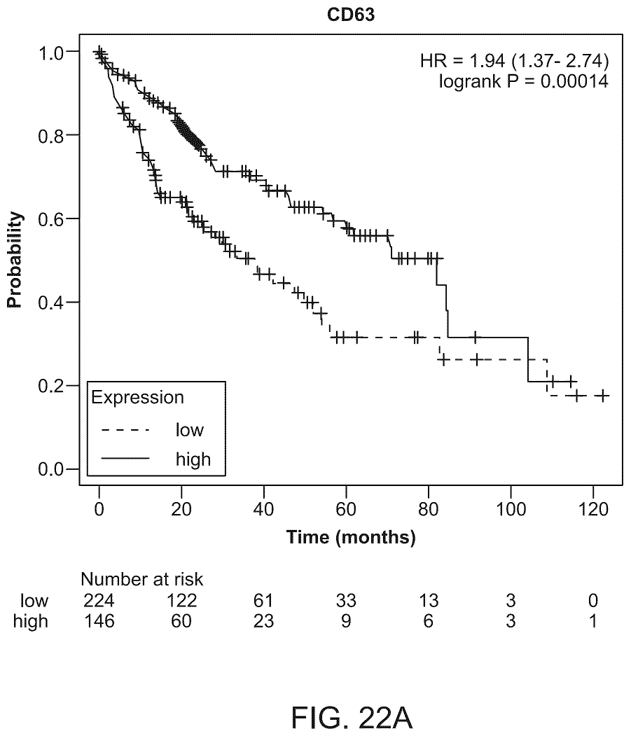

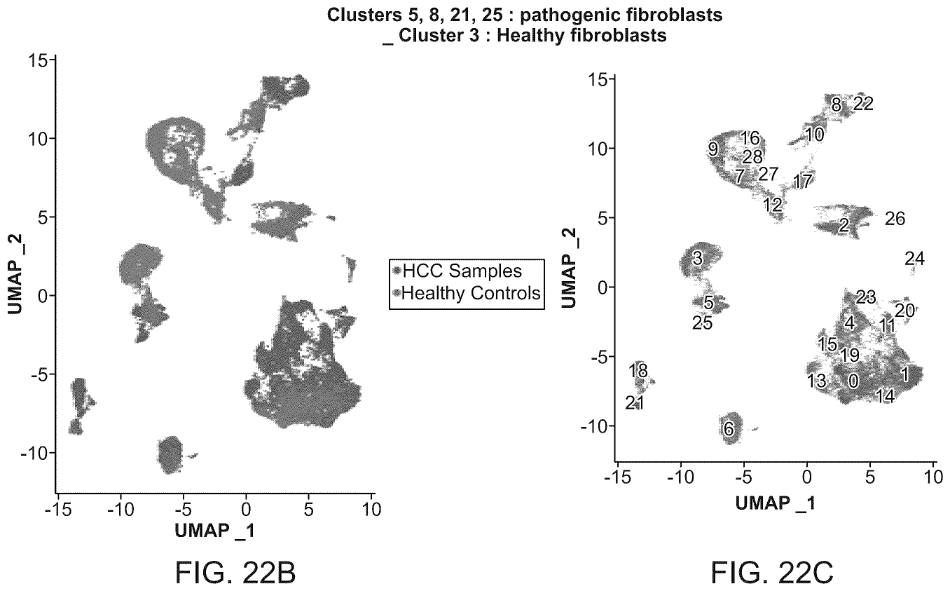

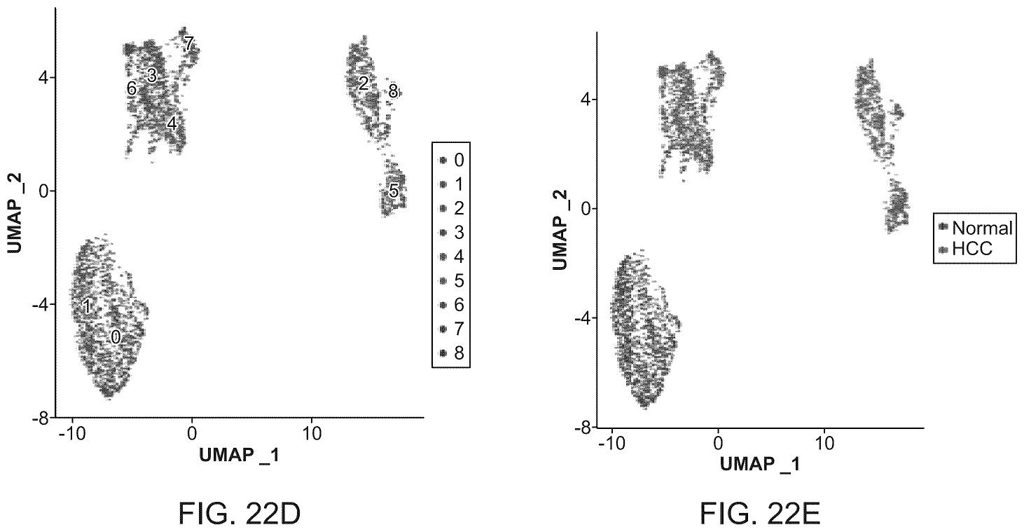

CD63 expression correlated with poor survival in cancer patients and is one of the most significantly overexpressed immune proteins in human patients with liver diseases. Using patient single cell transcriptomic data from 25 patients (>50,000 cells each) we compared the single cell populations identified between the healthy and diseased population. Using hepatocellular carcinoma as our disease model we clustered multiple populations of cell specific subtypes in both healthy and diseased liver (FIG. 22 A). Cell specific populations in HCC cluster differently than cellular subpopulations in healthy livers (FIG. 22 B). Of the cell specific clusters, clusters 5, 8, 21 and 25 are pathogenic fibroblast subclusters (FIG. 22 C). Cluster 3 comprises healthy fibroblasts. Re-clustering the selected fibroblast subclusters demonstrates clusters 2, 8 and 5 as highly pathogenic fibroblast clusters in liver cancer whereas clusters 1 and 0 are highly enriched fibroblasts in healthy livers (FIG. 22 D). UMAP indicated that cell population clusters were highly enriched in liver cancer and cell population clusters highly enriched in normal livers (FIG. 22 E). We further perturbed the individual proteins that are significantly enriched in the pathogenic clusters (green) and the healthy clusters (orange).

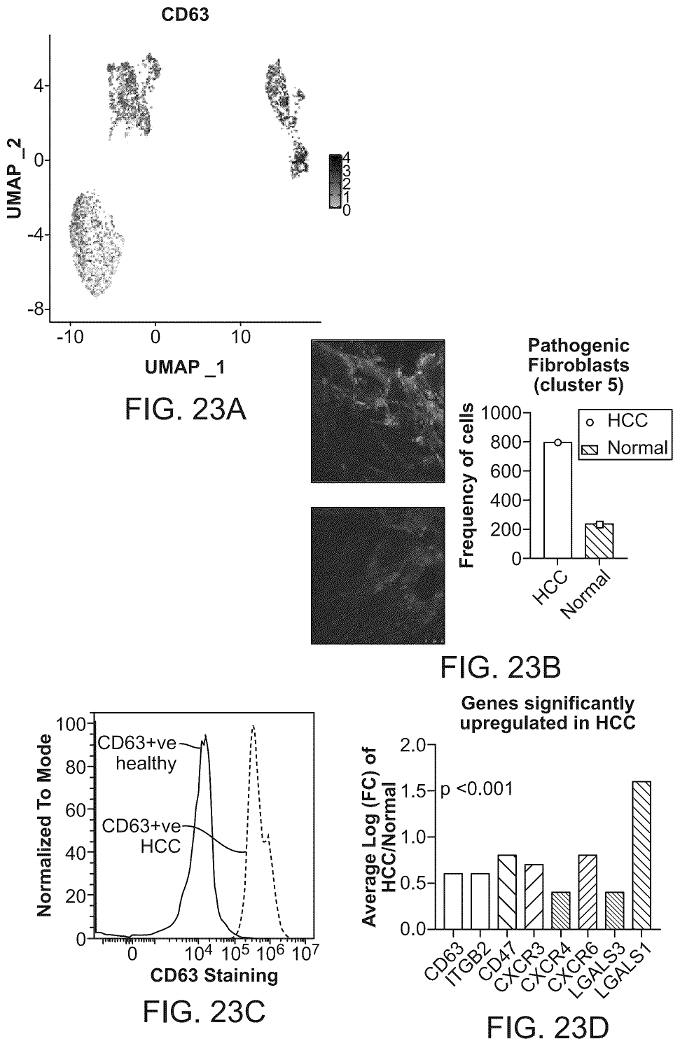

FIG. 23. CD63 was significantly upregulated in primary human liver tissue in HCC. To validate the highest gene enrichment obtained in cell population clusters highly enriched in liver cancer and cell population clusters highly enriched in normal livers, we demonstrate a UMAP indicating CD63 expression is the highest in the pathogenic fibroblast clusters in liver cancer. This enrichment is significantly higher than that obtained in normal healthy liver clusters. To identify the percent cell enrichment in each cluster, we analyzed the frequency of cells observed in the most pathogenic cluster (cluster 5; FIG. 23 B). High enrichment of pathogenic fibroblasts were demonstrated that are CD63 positive (˜800 cells) relative to CD63+ve cells in the normal liver (˜200 cells). After isolating primary fibroblasts from liver cancer patients, CD63 staining of primary cells reveals significant overexpression of CD63 in the primary patient fibroblasts. Flow cytometry validation further demonstrated this point with CD63 expression (FIG. 23 C) (Mean Fluorescence Intensity >2-fold higher in HCC samples compared to healthy control cells). Comparing across the most highly expressed genes in HCC, it was demonstrated CD63 as one of the most significantly enriched genes in liver cancer patients with expression levels comparable to known molecular targets of cancer like LGALS1, CD47 and CXCR3 (FIG. 23 D).

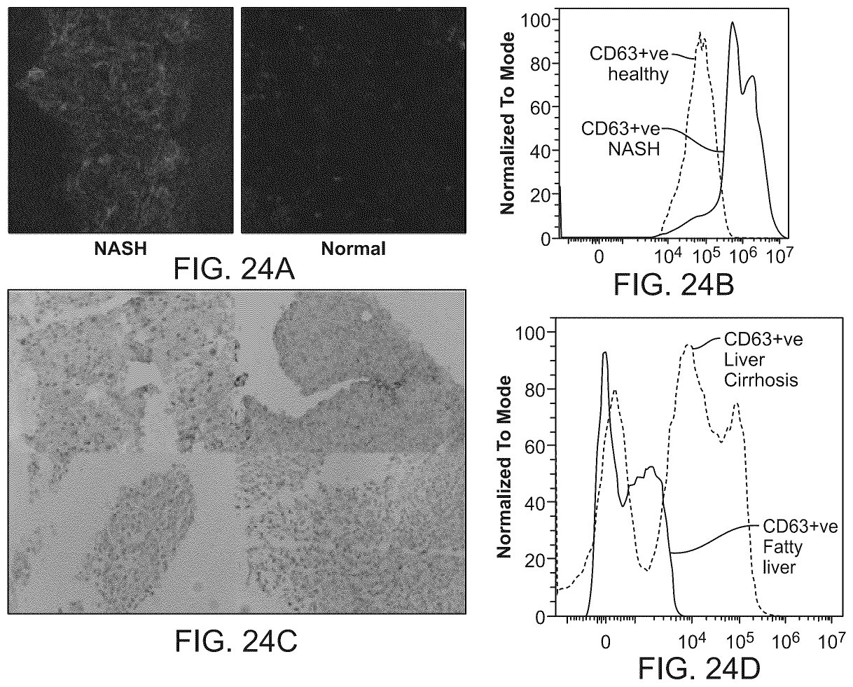

FIG. 24. CD63 is significantly enriched in other liver diseases like NASH, NAFLD and liver cirrhosis. To analyze the expression of CD63 in other liver pathologies like NASH, NAFLD and Liver cirrhosis, NASH human tissue was stained for CD63 expression. CD63 is highly expressed in NASH human tissue compared to healthy liver tissue (FIG. 24 A). Flow cytometry analyses further demonstrated that CD63 expression was significantly higher in NASH patient primary cells relative to normal liver cells (FIG. 24 B). To further evaluate CD63 expression both in NAFLD and cirrhotic liver, RNA scope was used to analyze CD63 expression which can de determined from probe expression (FIG. 24 C). CD63 expression is high in NAFLD and cirrhotic liver. This expression co-localized with FSP1 which is a known marker of fibrosis. These data describe the expression levels of CD63 which exhibit patterns similar to that of fibrotic liver markers. Finally, to evaluate the role of CD63 in the development of liver pathogenic diseases like liver cirrhosis, we used flow cytometric analyses was used to demonstrate that in fatty liver patient primary cells there is a population of cells that is CD63 positive but a large population of cells that are CD63 negative (FIG. 24 D). However, looking at primary cells from liver cirrhosis patients, it is evident that a majority of the cell populations highly express CD63. These results strongly suggest that CD63 could play an important role in the fatty liver progressing from a normal phenotype to a more aggressive pathogenic fibrotic phenotype that is observed in liver cirrhosis patients.

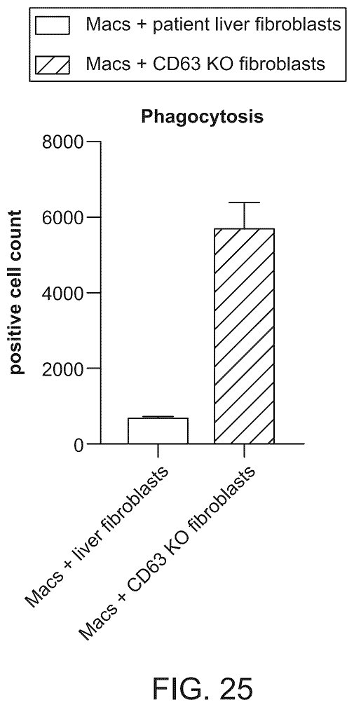

As shown in FIG. 25, genetic knock-out of CD63 increases phagocytosis of pathogenic liver fibroblasts. Genetically knocking out CD63 in pathogenic human fibroblasts increases the number of human macrophages when tested in a co-culture system staining for M2 macrophage marker CD204. Since phagocytosis is a prime mechanism through which several immune therapeutic antibodies target pathogenic diseases for treatment, it was believed that blocking CD63 is an essential treatment mechanism through which we can elicit an immune response against pathogenic fibroblasts in diseases like liver cirrhosis, NASH, HCC and NAFLD paired student t-test (p<0.001).
1. Bray F, Ferlay J, Soerjomataram I, Siegel R L, Torre L A, and Jemal A. Global cancer statistics 2018: GLOBOCAN estimates of incidence and mortality worldwide for 36 cancers in 185 countries. CA: A Cancer Journal for Clinicians. 2018; 68(6):394-424.
2. Tzouvelekis A, Spagnolo P, Bonella F, Vancheri C, Tzilas V, Crestani B, et al. Patients with IPF and lung cancer: diagnosis and management. Lancet Respir Med. 2018; 6(2):86-8.
3. Chen X, and Song E. Turning foes to friends: targeting cancer-associated fibroblasts. Nature Reviews Drug Discovery. 2019; 18(2):99-115.
4. Paz-Ares L, Dvorkin M, Chen Y, Reinmuth N, Hotta K, Trukhin D, et al. Durvalumab plus platinum-etoposide versus platinum-etoposide in first-line treatment of extensive-stage small-cell lung cancer (CASPIAN): a randomised, controlled, open-label, phase 3 trial. The Lancet. 2019; 394(10212):1929-39.
5. Arbour K C, and Riely G J. Systemic Therapy for Locally Advanced and Metastatic Non-Small Cell Lung Cancer. JAMA. 2019; 322(8):764.
6. Yu H, Boyle T A, Zhou C, Rimm D L, and Hirsch F R. PD-L1 Expression in Lung Cancer. Journal of Thoracic Oncology. 2016; 11(7):964-75.
7. Camidge D R, Kim H R, Ahn M-J, Yang J C-H, Han J-Y, Lee J-S, et al. Brigatinib versus Crizotinib in ALK-Positive Non-Small-Cell Lung Cancer. New England Journal of Medicine. 2018; 379(21):2027-39.
8. Prada I, and Meldolesi J. Binding and Fusion of Extracellular Vesicles to the Plasma Membrane of Their Cell Targets. Int J Mol Sci. 2016; 17(8):1296.
9. Kahlert C, and Kalluri R. Exosomes in tumor microenvironment influence cancer progression and metastasis. Journal of Molecular Medicine. 2013; 91(4):431-7.
10. Aaberg-Jessen C, Sorensen M D, Matos ALSA, Moreira J M, Brünner N, Knudsen A, et al. Co-expression of TIMP-1 and its cell surface binding partner CD63 in glioblastomas. BMC Cancer. 2018; 18(1):270.
11. Kaprio T, Hagström J, Andersson L C, and Haglund C. Tetraspanin CD63 independently predicts poor prognosis in colorectal cancer. Histol Histopathol. 2020:18209.
12. Koh H, An H, Jung J, and Song D. The prognostic significance of CD63 expression in patients with non-small cell lung cancer. Polish Journal of Pathology. 2019; 70(3):183-8.
13. Rorive S, Lopez X M, Maris C, Trepant A-L, Sauvage S, Sadeghi N, et al. TIMP-4 and CD63: new prognostic biomarkers in human astrocytomas. Modern Pathology. 2010; 23(10):1418-28.
14. Kwon M J, Seo J, Kim Y J, Kwon M J, Choi J Y, Kim T-E, et al. Prognostic significance of CD151 overexpression in non-small cell lung cancer. Lung Cancer. 2013; 81(1):109-16.
15. Cham L B, Torrez Dulgeroff L B, Tal M C, Adomati T, Li F, Bhat H, et al. Immunotherapeutic Blockade of CD47 Inhibitory Signaling Enhances Innate and Adaptive Immune Responses to Viral Infection. Cell Reports. 2020; 31(2):107494.
16. Advani R, Flinn I, Popplewell L, Forero A, Bartlett N L, Ghosh N, et al. CD47 Blockade by Hu5F9-G4 and Rituximab in Non-Hodgkin's Lymphoma. New England Journal of Medicine. 2018; 379(18):1711-21.
17. Jaiswal S, Jamieson C H M, Pang W W, Park C Y, Chao M P, Majeti R, et al. CD47 is upregulated on circulating hematopoietic stem cells and leukemia cells to avoid phagocytosis. Cell. 2009; 138(2):271-85.
18. Shen Y, Wang X, Lu J, Salfenmoser M, Wirsik N M, Schleussner N, et al. Reduction of Liver Metastasis Stiffness Improves Response to Bevacizumab in Metastatic Colorectal Cancer. Cancer Cell. 2020; 37(6):800-17.e7.
19. Berna, Pentcheva-Hoang T, Julienne, Zheng X, Wu C-C, Tyler, et al. Depletion of Carcinoma-Associated Fibroblasts and Fibrosis Induces Immunosuppression and Accelerates Pancreas Cancer with Reduced Survival. Cancer Cell. 2014; 25(6):719-34.
20. Pape J, Magdeldin T, Stamati K, Nyga A, Loizidou M, Emberton M, et al. Cancer-associated fibroblasts mediate cancer progression and remodel the tumouroid stroma. British Journal of Cancer. 2020.
21. Dzobo K, and Dandara C. Architecture of Cancer-Associated Fibroblasts in Tumor Microenvironment: Mapping Their Origins, Heterogeneity, and Role in Cancer Therapy Resistance. OMICS: A Journal of Integrative Biology. 2020; 24(6):314-39.
22. Wernig G, Chen S-Y, Cui L, Van Neste C, Tsai J M, Kambham N, et al. Unifying mechanism for different fibrotic diseases. Proceedings of the National Academy of Sciences. 2017.
23. Cui L, Chen S-Y, Lerbs T, Lee J-W, Domizi P, Gordon S, et al. Activation of JUN in fibroblasts promotes pro-fibrotic programme and modulates protective immunity. Nature Communications. 2020; 11(1).
24. Fahrmann J F, Grapov D, Phinney B S, Stroble C, Defelice B C, Rom W, et al. Proteomic profiling of lung adenocarcinoma indicates heightened DNA repair, antioxidant mechanisms and identifies LASP1 as a potential negative predictor of survival. Clinical Proteomics. 2016; 13(1).
25. Li G, Endsley M A, Somasunderam A, Gbota S L, Mbaka M I, Murray J L, et al. The dual role of tetraspanin CD63 in HIV-1 replication. Virology Journal. 2014; 11(1):23.
26. Schulze U, Brast S, Grabner A, Albiker C, Snieder B, Holle S, et al. Tetraspanin CD63 controls basolateral sorting of organic cation transporter 2 in renal proximal tubules. 2016:fj.201600901R.
1. Raghu, G., et al., An official ATS/ERS/JRS/ALAT statement: idiopathic pulmonary fibrosis: evidence-based guidelines for diagnosis and management. American journal of respiratory and critical care medicine, 2011. 183(6): p. 788-824.
2. Dzobo, K. and C. Dandara, Architecture of Cancer-Associated Fibroblasts in Tumor Microenvironment: Mapping Their Origins, Heterogeneity, and Role in Cancer Therapy Resistance. OMICS: A Journal of Integrative Biology, 2020. 24(6): p. 314-339.
3. Cox, T. R., The matrix in cancer. Nature Reviews Cancer, 2021. 21(4): p. 217-238.
4. Klemm, F. and J. A. Joyce, Microenvironmental regulation of therapeutic response in cancer. Trends in Cell Biology, 2015. 25(4): p. 198-213.
5. Gonzalez, H., C. Hagerling, and Z. Werb, Roles of the immune system in cancer: from tumor initiation to metastatic progression. Genes & development, 2018. 32(19-20): p. 1267-1284.
6. Seidel, J. A., A. Otsuka, and K. Kabashima, Anti-PD-1 and Anti-CTLA-4 Therapies in Cancer: Mechanisms of Action, Efficacy, and Limitations. Frontiers in oncology, 2018. 8: p. 86-86.
7. Hellmann, M. D., et al., Nivolumab plus Ipilimumab in Lung Cancer with a High Tumor Mutational Burden. New England Journal of Medicine, 2018. 378(22): p. 2093-2104.
8. Advani, R., et al., CD47 Blockade by Hu5F9-G4 and Rituximab in Non-Hodgkin's Lymphoma. New England Journal of Medicine, 2018. 379(18): p. 1711-1721.
9. Jaiswal, S., et al., CD47 is upregulated on circulating hematopoietic stem cells and leukemia cells to avoid phagocytosis. Cell, 2009. 138(2): p. 271-285.
10. Kojima, Y., et al., CD47-blocking antibodies restore phagocytosis and prevent atherosclerosis. Nature, 2016. 536(7614): p. 86-90.
11. Lerbs, T., et al., CD47 prevents the elimination of diseased fibroblasts in scleroderma. JCI Insight, 2020. 5(16).
12. Wernig, G., et al., Unifying mechanism for different fibrotic diseases. Proceedings of the National Academy of Sciences, 2017.
13. Sculier, J. P., et al., The impact of additional prognostic factors on survival and their relationship with the anatomical extent of disease expressed by the 6th Edition of the TNM Classification of Malignant Tumors and the proposals for the 7th Edition. J Thorac Oncol, 2008. 3(5): p. 457-66.
14. Camidge, D. R., et al., Brigatinib versus Crizotinib in ALK-Positive Non-Small-Cell Lung Cancer. New England Journal of Medicine, 2018. 379(21): p. 2027-2039.
15. Howlader, N., et al., The Effect of Advances in Lung-Cancer Treatment on Population Mortality. New England Journal of Medicine, 2020. 383(7): p. 640-649.
16. Siegel, R. L., et al., Cancer Statistics, 2021. CA Cancer J Clin, 2021. 71(1): p. 7-33.
17. Gettinger, S., et al., Five-Year Follow-Up of Nivolumab in Previously Treated Advanced Non-Small-Cell Lung Cancer: Results From the CA209-003 Study. Journal of Clinical Oncology, 2018. 36(17): p. 1675-1684.
18. Fares, C. M., et al., Mechanisms of Resistance to Immune Checkpoint Blockade: Why Does Checkpoint Inhibitor Immunotherapy Not Work for All Patients? American Society of Clinical Oncology Educational Book, 2019(39): p. 147-164.
19. Lu, R.-M., et al., Development of therapeutic antibodies for the treatment of diseases. Journal of Biomedical Science, 2020. 27(1): p. 1.
20. Carter, P. J. and G. A. Lazar, Next generation antibody drugs: pursuit of the ‘high-hanging fruit’. Nature Reviews Drug Discovery, 2018. 17(3): p. 197-223.
21. Reichert, J. M., Antibodies to watch in 2017. MAbs, 2017. 9(2): p. 167-181.
22. Lander, E. S., et al., Initial sequencing and analysis of the human genome. Nature, 2001. 409(6822): p. 860-921.
23. Hemler, M. E., Tetraspanin proteins promote multiple cancer stages. Nature Reviews Cancer, 2014. 14(1): p. 49-60.
24. Brosseau, C., et al., CD9 Tetraspanin: A New Pathway for the Regulation of Inflammation? Frontiers in Immunology, 2018. 9.
25. Vences-Catalán, F., et al., CD81 as a tumor target. Biochemical Society Transactions, 2017. 45(2): p. 531-535.
26. Khushman, M.d., et al., Exosomal Markers (CD63 and CD9) Expression Pattern Using Immunohistochemistry in Resected Malignant and Nonmalignant Pancreatic Specimens.
Pancreas, 2017. 46(6): p. 782-788.
27. Kwon, M. J., et al., Prognostic significance of CD151 overexpression in non-small cell lung cancer. Lung Cancer, 2013. 81(1): p. 109-116.
28. Kaprio, T., et al., Tetraspanin CD63 independently predicts poor prognosis in colorectal cancer. Histol Histopathol, 2020: p. 18209.
29. Lai, X., et al., Decreased expression of CD63 tetraspanin protein predicts elevated malignant potential in human esophageal cancer. Oncology Letters, 2017. 13(6): p. 4245-4251.
30. Deng, Y., et al., Tetraspanins: Novel Molecular Regulators of Gastric Cancer. Frontiers in oncology, 2021. 11: p. 702510-702510.
31. Chen, Z., et al., Down-regulation of TM4SF is associated with the metastatic potential of gastric carcinoma TM4SF members in gastric carcinoma. World J Surg Oncol, 2011. 9: p. 43.
32. Györffy, B., et al., Online Survival Analysis Software to Assess the Prognostic Value of Biomarkers Using Transcriptomic Data in Non-Small-Cell Lung Cancer. PLOS ONE, 2013. 8(12): p. e82241.

What is claimed is:

1. A method for prevention or treatment of fibrosis in a mammalian patient, the method comprising:
administering a dose of an anti-CD63 agent to the mammalian patient effective to prevent or reduce fibrosis.

2. A method of treating cancer in a mammalian patient, the method comprising: administering a dose of an anti-CD63 agent to the patient effective to reduce the cancer cells.

3. The method of claim 1 or claim 2, wherein phagocytosis of targeted cancer cells or fibroblasts is increased.

4. The method of any of claims 1-3, wherein the cancer is selected from lung cancer, liver cancer, osteosarcoma and sarcoma.

5. The method of any of claims 1-4, wherein the fibrosis is selected from pulmonary fibrosis, skin fibrosis, liver cirrhosis, non-alcoholic steatohepatitis (NASH), non-alcoholic fatty liver disease (NHFLD), and kidney fibrosis wherein the kidney fibrosis is caused by hypertension, diabetes, obstruction, or infection.

6. The method of any of claims 1-5, wherein the anti-CD63 agent is an antibody that neutralizes CD63.

7. The method of claim 6, wherein the antibody is humanized or chimeric.

8. The method of any of claims 1-5, wherein the anti-CD63 agent is a soluble CD63 polypeptide.

9. The method of any of claims 1-5, wherein the anti-CD63 agent is a therapeutic gene therapy comprising a CRISPR/Cas9 system and at least one single guide RNA (sgRNA) directed to a CD63 gene.

10. The method of claim 9, wherein the therapeutic gene therapy comprises two or more single guide RNAs (sgRNAs) directed to a CD63 gene.

11. The method of claim 9-10, wherein the sequence of the sgRNA is selected from SEQ ID NO: 1, 2, or a combination thereof.

12. The method of any of claims 1-11, wherein the anti-CD63 agent is administered locally at the site of the cancer or fibrotic tissue.

13. The method of any of claims 1-11, wherein the anti-CD63 agent is administered systemically.

14. The method of any of claims 1-13, wherein the individual has been diagnosed by the method comprising:
determining the presence of elevated levels of CD63 in a patient biological sample.

15. The method of any of claims 1-14, wherein the individual has been analyzed after prevention or treatment by the method comprising:
determining the extent of fibrosis in the patient or in a patient biological sample.


SEQUENCE LISTING (2 sequences)

SEQ ID NO: 1 - DNA, 20 bp, Artificial Sequence
  sgRNA for human CD63
caaccacactgcttcgatcc

SEQ ID NO: 2 - DNA, 20 bp, Artificial Sequence
  sgRNA for human CD63
cccactgcgatgatgaccac